(19) United States
(12) Patent Application Publication
(10) Pub. No.: US 2006/0020022 A1
(43) Pub. Date: Jan. 26, 2006

(54) Enantioselective separation method

(75) Inventors: Brian P. Chekal, Kalamazoo, MI (US); Ernst Freund, Aarau (CH); Charles Minard Grill, Mystic, CT (US); Elke Huthmann, Buchs (CH); Markus Juza, Buchs (CH); Vera Leshchinskaya, West Windsor, NJ (US); Mark Thomas Maloney, Kalamazoo, MI (US); Lawrence Marvin Miller JR., Stow, MA (US); Ying Wang, Plymouth, MN (US); Ming Zeng, East Lyme, CT (US)

Correspondence Address: WARNER-LAMBERT COMPANY, 2800 PLYMOUTH RD, ANN ARBOR, MI, 48105, US

(73) Assignee: PHARMACIA & UPJOHN COMPANY LLC

(21) Appl. No.: 11/187,464

(22) Filed: Jul. 22, 2005

Related U.S. Application Data
(60) Provisional application No. 60/590,516, filed on Jul. 23, 2004.

(51) Int. Cl. C07D 311/04; A61K 31/353

(52) U.S. Cl. 514/456; 549/403

(57) ABSTRACT

This invention relates to a method for separating enantiomers of a substituted 2-trifluoromethyl-2H-chromene-3-carboxylic acid or ester, a substituted 2-trifluoromethyl-1,2-dihydro-quinoline-3-carboxylic acid or ester, a substituted 2-trifluoromethyl-2H-thiochromene-3-carboxylic acid or ester, a substituted 3-trifluoromethyl-3,4-dihydro-naphthalene-2-carboxylic acid or ester, or a pharmaceutically acceptable salt of the acids or esters, using enantioselective fractional crystallization, enantioselective high performance liquid chromatography, enantioselective steady state recycling chromatography, or enantioselective multicolumn chromatography.

### CROSS-REFERENCE TO RELATED APPLICATIONS

This application claims priority from U.S. Provisional Patent Application No. 60/590,516 filed Jul. 23, 2004.

### FIELD OF THE INVENTION

This invention relates to a method for separating enantiomers of a substituted 2-trifluoromethyl-2H-chromene-3-carboxylic acid or ester, a substituted 2-trifluoromethyl-1,2-dihydro-quinoline-3-carboxylic acid or ester, a substituted 2-trifluoromethyl-2H-thiochromene-3-carboxylic acid or ester, a substituted 3-trifluoromethyl-3,4-dihydro-naphthalene-2-carboxylic acid or ester, or a pharmaceutically acceptable salt of the acids or esters, using enantioselective fractional crystallization, enantioselective high performance liquid chromatography, enantioselective steady state recycling chromatography, or enantioselective multicolumn chromatography.

### BACKGROUND OF THE INVENTION

Multicolumn chromatography includes the methods known as asynchronous multicolumn chromatography and simulated moving bed (“SMB”) chromatography. SMB chromatography was invented in the 1960's and reported by Broughton, D. B., et al., Chem. Eng. Process, 1970; 66(9):70. SMB chromatography has been subsequently adapted for enantioselective separations of enantiomers of pharmaceutically active compounds and related chiral intermediates. Illustrative pharmaceutical industry applications are described in U.S. Pat. Nos. 5,928,515; 5,939,552; 6,107,492; 6,130,353; 6,455,736; 6,458,955; and PCT International Patent Application Publication Numbers WO 99/47531; WO 99/57089; WO 03/006449; WO 03/016245; WO 03/021355; and WO 03/051867.

Asynchronous multicolumn chromatography includes VARICOL® multicolumn chromatography, which is described by Ludemann-Hombourger, O., Nicoud R. M., and Bailly M., “The VARICOL process: a new multicolumn continuous chromatographic process,” Sep. Sci. & Techno., 2000; 35(12): 1827-1860 and further applied by Ludemann-Hombourger, O., Pigorini G., Nicoud R. M., Ross D. S., and Terfloth G., “Application of the ‘VARICOL’ process to the separation of the isomers of the SB-553261 racemate,” Journal of Chromatography A, 2002; 947:59-68.

Steady state recycling chromatography is described in U.S. Pat. Nos. 5,630,943 (two column mode) and 6,063,284 (single column mode).

Substituted 2-trifluoromethyl-2H-chromene-3-carboxylic acids and derivatives thereof are described in U.S. Pat. No. 6,034,256; 6,077,850; 6,218,427; or 6,271,253 or U.S. patent application Ser. No. 10/801,446 or 10/801,429. The derivatives thereof include compounds such as esters thereof, substituted 2-trifluoromethyl-1,2-dihydro-quinoline-3-carboxylic acids or esters, substituted 2-trifluoromethyl-2H-thiochromene-3-carboxylic acids or esters, and substituted 3-trifluoromethyl-3,4-dihydro-naphthalene-2-carboxylic acids or esters, and pharmaceutically acceptable salts thereof. The substituted 2-trifluoromethyl-2H-chromene-3-carboxylic acids and derivatives thereof each have a chiral center at the 2-position of the chromene, quinoline, or thiochromene and the 3-position of the 3,4-dihydro-napthalene. The ring carbon atom of the chiral center is bonded to four functional groups. Two of these four functional groups are a hydrogen atom and a R¹ group or trifluoromethyl (“CF₃”) group. The other two of these four functional groups are the group X as defined below and the sp² carbon atom at the 3-position of the chromene, quinoline, and thiochromene or the sp² carbon atom at the 2-position of the 3,4-dihydro-napthalene.

The chiral substituted 2-trifluoromethyl-2H-chromene-3-carboxylic acids and derivatives thereof comprise enantiomers having either the (S)- or the (R)-configuration of the four functional groups that are bonded to the carbon atom of the chiral center. The (S)- and (R)-configurations represent the three-dimensional orientation of the four functional groups about the chiral center carbon atom. The enantiomers having either the (S)- or the (R)-configuration about the carbon atom of the chiral center bonded to the R¹ group or 2-trifluoromethyl group are referred to herein as (2S)- and (2R)-enantiomers, respectively, or the (3S)-and (3R)-enantiomers in the case of the 3,4-dihydro-naphthalene derivatives. The (2S)-enantiomer is the antipode (i.e., non-superimposable mirror image) of the (2R)-enantiomer and vice versa. The (3S)-enantiomer is the antipode of the (3R)-enantiomer and vice versa.

Generally, the (2S)-, (2R)-, (3S)- and (3R)-enantiomers of the substituted 2-trifluoromethyl-2H-chromene-3-carboxylic acids and derivatives thereof are physically and chemically identical to each other except for how they rotate plane-polarized light and how they interact with other chiral molecules such as each other and biological enzymes, receptors, and the like. The (2S)-, (2R) (3S)- and (3R)-enantiomers of the substituted 2-trifluoromethyl-2H-chromene-3-carboxylic acids and derivatives thereof are more potent inhibitors of the enzyme cyclooxygenase-2 (“COX-2”) than of the enzyme cyclooxygenase-1 (“COX-1”).

These enantiomers represent a new generation of “COX-2 inhibitors.” Typically for a particular compound, either the (2S)- or the (2R)-enantiomer (or the (3S)- or the (3R)-enantiomer in the case of 3,4-dihydro-naphthalene derivatives) exhibits (a) more potency for COX-2, (b) greater selectivity for COX-2-over COX-1, or (c) different metabolic profiles using liver microsome preparations than that for the other of the (2S)- and (2R)-enantiomers (or the (3S)- or the (3R)-enantiomers). Sometimes it is the (2S)-enantiomer (or (3S)-enantiomer) and other times it is the (2R)-enantiomer (or (3R)-enantiomer), depending upon the particular compound being considered, that has the more potent or selective inhibitory activity or superior metabolic profile. Depending upon the potency or selectivity inhibitory activity, metabolic profile, or other biological activities of the particular compound being considered, sometimes the (2S)-enantiomer (or (3S)-enantiomer) is preferred for drug development and other times the (2R)-enantiomer (or (3R)-enantiomer) is preferred.

The substituted 2-trifluoromethyl-2H-chromene-3-carboxylic acids and derivatives thereof typically are synthesized as mixtures (racemic or otherwise) of their enantiomers because a commercially better, direct enantioselective synthesis has not been devised yet. In order to be able to make multi-kilogram quantities of a particular enantiomer substituted 2-trifluoromethyl-2H-chromene-3-carboxylic acid, or derivative thereof, widely available as a pharmaceutical agent to patients in need of treatment with a COX-2 inhibitor, a mixture of the enantiomer and its antipode possibly could be separated by enantioselective fractional crystallization with a chiral auxiliary and/or enantioselective multicolumn chromatography over chiral stationary phase. The goal of these enantioselective purification methods is to ultimately produce the more desired enantiomer in high (preferably ≧99.0%) enantiomeric excess (“e.e.”), which is the relative percent of one enantiomer in excess of its antipode and ignoring any other impurities (e.g., a mixture containing 99.5% of an enantiomer and 0.5% of its antipode has an e.e. of 99.0% and a mixture containing 90% of an enantiomer and 10% of its antipode has an e.e. of 80%).

The method of enantioselective purification of the enantiomers may include enantioselective fractional crystallization, enantioselective chromatography, and/or an optional step that converts a less preferred enantiomer to a new mixture of enantiomers and a subsequent recycle step that separates the new mixture of enantiomers, thereby producing from the less preferred enantiomer additional quantities of the more preferred enantiomer.

Enantioselective fractional crystallization of a racemic mixture of certain substituted 2-trifluoromethyl-2H-chromene-3-carboxylic acids and a certain substituted 2-trifluoromethyl-1,2-dihydro-quinoline-3-carboxylic acid with a chiral auxiliary has been described in Examples 66 to 68 and 172 of U.S. Pat. No. 6,077,850 for the purpose of preparing the corresponding (2S)-enantiomers. Yields were 45%, 59%, 30%, and 10%, respectively, after multiple crystallizations and extractions. Optical purities, determined by derivatizing the (2S)-carboxylic acids by reaction with (trimethylsilyl)diazomethane to give the corresponding trimethylsilyl ester, and subjecting the silyl ester to enantioselective chromatography, were greater than 90% enantiomeric excess (“e.e.”).

Accordingly, there is a need for cost-effective method of efficiently separating enantiomers of the substituted 2-trifluoromethyl-2H-chromene-3-carboxylic acids and derivatives thereof, that produces a preferred enantiomer in high yield (e.g., >80%) and enantiomeric excess (e.g., at least 95% e.e.). The method of the present invention relates to enantioselective fractional crystallization and enantioselective high performance liquid chromatography, enantioselective steady state chromatography, and enantioselective multicolumn chromatography of a substituted 2-trifluoromethyl-2H-chromene-3-carboxylic acid or derivative thereof, that efficiently and cost effectively produces either separately purified (2R)- and (2S)-enantiomers (or (3R)- and (3S)-enantiomers in the case of 3,4-dihydro-naphthalene derivatives), or a purified single (2R)- or (2S)-enantiomer (or (3R)- or (3S)-enantiomer in the case of 3,4-dihydro-naphthalene derivatives), depending on what is desired, in satisfactory yield and enantiomeric excess.

### BRIEF SUMMARY OF THE INVENTION

The present invention relates to a method for enantioselectively separating enantiomers of a substituted 2-trifluoromethyl-2H-chromene-3-carboxylic acid or derivative thereof. One aspect of this invention is a method for separating enantiomers of a substituted 2-trifluoromethyl-2H-chromene-3-carboxylic acid or derivative thereof, the method comprising:
(a) introducing a mixture of the enantiomers to a chiral stationary phase; and
(b) eluting at least one of the enantiomers with a mobile phase;
wherein the substituted 2-trifluoromethyl-2H-chromene-3-carboxylic acid or derivative thereof is a compound of Formulas I″, I′, I, or II 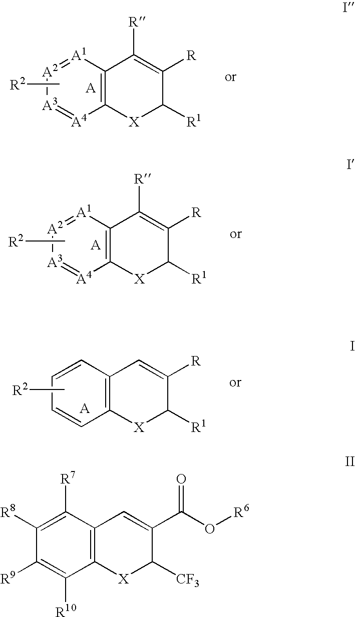
for Formula I″:
wherein X is selected from O, S, CR^{c}R^{b} and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₁-C₆-alkyl and C₂-C₆-alkenyl; wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I′:
wherein X is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₂-C₆-alkynyl and C₂-C₆-alkenyl;
wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon; or wherein R² together with ring A forms a radical selected from naphthyl, quinolyl, isoquinolyl, quinolizinyl, quinoxalinyl and dibenzofuryl;
for Formula I:
wherein X is selected from O or S or NR^{a};
wherein R^{a} is alkyl;
wherein R is selected from carboxyl, aminocarbonyl, alkylsulfonylaminocarbonyl and alkoxycarbonyl;
wherein R¹ is selected from haloalkyl, alkyl, aralkyl, cycloalkyl and aryl optionally substituted with one or more radicals selected from alkylthio, nitro and alkylsulfonyl; and
wherein R² is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R² together with ring A forms a naphthyl radical;
for Formula II:
wherein X is selected from O, S, and NH;
wherein R⁶ is H or alkyl; and
wherein R⁷, R⁸, R⁹, and R¹⁰ independently are selected from H, alkenyl, alkoxy, alkoxyalkyl, alkoxycarbonylalkyl, alkyl, alkylamino, alkylcarbonyl, alkylheteroaryl, alkylsulfonylalkyl, alkylthio, alkynyl, aminocarbonylalkyl, aryl, arylalkenyl, arylalkoxy, arylalkyl, arylalkylamino, arylalkynyl, arylcarbonyl, aryloxy, cyano, dialkylamino, halo, haloalkoxy, haloalkyl, heteroaryl, heteroarylalkoxy, heteroarylcarbonyl, hydroxy and hydroxyalkyl; wherein each of aryl, wherever it occurs, is independently substituted with one to five substituents selected from the group consisting of alkyl, alkoxy, alkylamino, cyano, halo, haloalkyl, hydroxy, and nitro.

Another aspect of this invention is any one of the above or below methods for separating enantiomers of a substituted 2-trifluoromethyl-2H-chromene-3-carboxylic acid or derivative thereof, the method further comprising a step of monitoring the eluate produced in the eluting step for at least one of the enantiomers.

Another aspect of this invention is any one of the above or below methods for separating enantiomers of a substituted 2-trifluoromethyl-2H-chromene-3-carboxylic acid or derivative thereof, the method further comprising a step of isolating in a form that is substantially free of mobile phase, at least one of the separated enantiomers.

Another aspect of this invention is any one of the above or below methods for separating enantiomers of a substituted 2-trifluoromethyl-2H-chromene-3-carboxylic acid or derivative thereof, wherein the mixture of the enantiomers comprises a substituted 2-trifluoromethyl-2H-chromene-3-carboxylic acid, a substituted 2-trifluoromethyl-1,2-dihydro-quinoline-3-carboxylic acid, a substituted 2-trifluoromethyl-2H-thiochromene-3-carboxylic acid, or a substituted 3-trifluoromethyl-3,4-dihydro-naphthalene-2-carboxylic acid and the mobile phase is:
a single polar solvent;
a solution comprising a polar solvent and an acidic solvent wherein the polar solvent is at least 99% volume/volume of the solution and the acidic solvent is less than 1% volume/volume of the solution; or
a solution comprising a polar solvent, an acidic solvent, and a nonpolar solvent wherein the polar solvent is less than or equal to 50% volume/volume of the mixture, the acidic solvent is less than 1% volume/volume of the solution and the nonpolar solvent is greater than 50% volume/volume of the solution.

Another aspect of this invention is any one of the above or below methods for separating enantiomers of a substituted 2-trifluoromethyl-2H-chromene-3-carboxylic acid or derivative thereof, wherein the method comprises enantioselective steady state recycling chromatography or enantioselective multicolumn chromatography.

Another aspect of this invention is any one of the above or below methods for separating enantiomers of a substituted 2-trifluoromethyl-2H-chromene-3 -carboxylic acid or derivative thereof, the method further comprising a step of subjecting at least one of the separated enantiomers produced in the eluting step to enantioselective fractional crystallization.

Another aspect of this invention is any one of the above or below methods for separating enantiomers of a substituted 2-trifluoromethyl-2H-chromene-3-carboxylic acid or derivative thereof, wherein the mixture of the enantiomers comprises a compound of Formula II wherein X is 0 and R⁶ is H.

Another aspect of this invention is any one of the above or below methods for separating enantiomers of a substituted 2-trifluoromethyl-2H-chromene-3-carboxylic acid or derivative thereof, wherein the mixture of the enantiomers comprises:
(R)- and (S)-6-chloro-7-tert-butyl-2-trifluoromethyl-2H-chromene-3-carboxylic acid.

Another aspect of this invention is any one of the above or below methods for separating enantiomers of a substituted 2-trifluoromethyl-2H-chromene-3-carboxylic acid or derivative thereof, wherein the mixture of the enantiomers comprises:
(R)- and (S)-6-chloro-8-methyl-2-trifluoromethyl-2H-chromene-3-carboxylic acid;
(R)- and (S)-6-chloro-5,7-dimethyl-2-trifluoromethyl-2H-chromene-3-carboxylic acid;
(R)- and (S)-6,8-dimethyl-2-trifluoromethyl-2H-chromene-3-carboxylic acid; or
(R)- and (S)-8-ethyl-6-trifluoromethoxy-2-trifluoromethyl-2H-chromene-3-carboxylic acid.

Another aspect of this invention is a method for separating enantiomers of a substituted 2-trifluoromethyl-2H-chromene-3-carboxylic acid or derivative thereof, the method comprising:
(a) subjecting a mixture of the enantiomers to enantioselective fractional crystallization to give crystals and mother liquor; and
(b) isolating at least one of the enantiomers from the crystals or mother liquor;
wherein the substituted 2-trifluoromethyl-2H-chromene-3-carboxylic acid or derivative thereof is a compound of Formulas I″, I′, I, or II 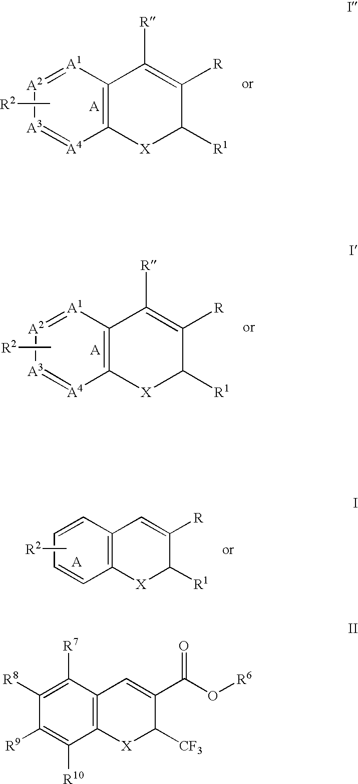
wherein:
for Formula I″, R¹, R, R″, R², A, A¹, A², A³, A⁴, and X are as they are defined above for Formula I″;
for Formula I′, R¹, R, R″, R², A, A¹, A², A³, A⁴, and X are as they are defined for Formula I′;
for Formula I, R¹, R, R″, R², A, A¹, A², A³, A⁴, and X are as they are defined for Formula I;
for Formula II, X, R⁶, R⁷, R⁸, R⁹, and R¹⁰ are as they are defined above for Formula II.

Another aspect of this invention is the above method for separating enantiomers of a substituted 2-trifluoromethyl-2H-chromene-3-carboxylic acid or derivative thereof by enantioselective fractional crystallization, wherein the crystals comprise a (S)-(−)-α-methylbenzylamine, (−)-cinchonidine, (S)-(−)-2 -amino-3-phenyl-1-propanol, (+)-brucine, (1R,2S)-2-amino-1,2-diphenyl ethanol, (R)-(+)-4-diphenylmethyl-2-oxozolidinone, (1R,2S)-(+)-cis-[2-(benzylamine)cyclohexyl]methanol, (+)-quinine, (+)-cinchonine, L-phenylalaninol, (R)-(−)-2-amino-1-butanol, (R)-(−)-phenylglycinol, (1R,2R)-(+)-1,2-diphenylethylenediamine, (1S,2R)-(+)-norephedrine, (1R,2S)-(−)-N-methylephedrine, (1R,2S)-(−)-ephedrine, (+)-quinidine, (1R,2S)-(+)-1-amino-2-indanol, (1R,2R)-(−)-2-amino-1-(4-nitrophenyl)-1,3-propanediol, (R)-(+)-N-benzyl-α-methylbenzylamine, (+)-strychnine, (+)-dehydroabietylamine, (+)-amphetamine, (+)-deoxyphedrine, (+)-chloramphenicol intermediate, (+)-1-(1-napthyl)ethylamine, (R)-(+)-α-methylbenzylamine, (+)-cinchonidine, (R)-(+)-2-amino-3-phenyl-1-propanol, (−)-brucine, (1S,2R)-2-amino-1,2-diphenyl ethanol, (S)-(−)-4-diphenylmethyl-2-oxozolidinone, (1S,2R)-(−)-cis-[2-(benzylamine)cyclohexyl]methanol, (−)-quinine, (−)-cinchonine, D-phenylalaninol, (S)-(+)-2-amino-1-butanol, (S)-(+)-phenylglycinol, (1S,2S)-(−)-1,2-diphenylethylenediamine, (1R,2S)-(−)-norephedrine, (1S,2R)-(+)-N-methylephedrine, (1S,2R)-(+)-ephedrine, (−)-quinidine, (1S,2R)-(−)-1-amino-2-indanol, (1S,2S)-(+)-2-amino-1-(4-nitrophenyl)-1,3-propanediol, (S)-(−)-N-benzyl-α-methylbenzylamine, (−)-strychnine, (−)-dehydroabietylamine, (−)-amphetamine, (−)-deoxyphedrine, (−)-chloramphenicol intermediate, or (−)-1-(1-napthyl)ethylamine salt of at least one enantiomer.

Another aspect of this invention is the above method for separating enantiomers of a substituted 2-trifluoromethyl-2H-chromene-3-carboxylic acid or derivative thereof by enantioselective fractional crystallization, wherein the crystals comprise:
(S)-6-chloro-8-methyl-2-trifluoromethyl-2H-chromene-3-carboxylic acid (S)-(−)-N-benzyl-α-methylbenzylamine salt;
(S)-6-chloro-5,7-dimethyl-2-trifluoromethyl-2H-chromene-3-carboxylic acid (+)-cinchonine salt;
(S)-6,8-dimethyl-2-trifluoromethyl-2H-chromene-3-carboxylic acid (S)-(−)-N-benzyl-α-methylbenzylamine salt;
(S)-8-ethyl-6-trifluoromethoxy-2-trifluoromethyl-2H-chromene-3-carboxylic acid (S)-(−)-N-benzyl-α-methylbenzylamine salt;
(R)-6-chloro-8-methyl-2-trifluoromethyl-2H-chromene-3-carboxylic acid
(R)-(+)-N-benzyl-α-methylbenzylamine salt;
(R)-6-chloro-5,7-dimethyl-2-trifluoromethyl-2H-chromene-3-carboxylic acid (−)-cinchonine salt;
(R)-6,8-dimethyl-2-trifluoromethyl-2H-chromene-3-carboxylic acid (R)-(+)-N-benzyl-α-methylbenzylamine salt; or
(R)-8-ethyl-6-trifluoromethoxy-2-trifluoromethyl-2H-chromene-3-carboxylic acid (R)-(+)-N-benzyl-α-methylbenzylamine salt.

Another aspect of this invention is the above method for separating enantiomers of a substituted 2-trifluoromethyl-2H-chromene-3-carboxylic acid or derivative thereof by enantioselective fractional crystallization, wherein the crystals comprise:
(R)-6-chloro-7-tert-butyl-2-trifluoromethyl-2H-chromene-3-carboxylic acid (+)-cinchonine salt;
(S)-6-chloro-7-tert-butyl-2-trifluoromethyl-2H-chromene-3-carboxylic acid (−)-cinchonine salt;
(S)-6-chloro-7-tert-butyl-2-trifluoromethyl-2H-chromene-3-carboxylic acid L-phenylalaninol salt; or
(R)-6-chloro-7-tert-butyl-2-trifluoromethyl-2H-chromene-3-carboxylic acid D-phenylalaninol salt.

### BRIEF DESCRIPTION OF THE DRAWINGS

FIG. 1 is a HPLC chromatogram of a batch enantioselective separation of a mixture of (R)- and (S)-6-chloro-7-tert-butyl-2-trifluoromethyl-2H-chromene-3-carboxylic acid.

FIG. 2 is an internal profile for an enantioselective SMB chromatography for the separation of (R)- and (S)-6-chloro-7-tert-butyl-2-trifluoromethyl-2H-chromene-3-carboxylic acid that plots True Moving Bed (“TMB”) Equivalent Position for an 8-column pathway on the x-axis and Integrated Absorbance on the y-axis.

### DETAILED DESCRIPTION OF THE INVENTION

One aspect of this invention is a method for separating enantiomers of a substituted 2-trifluoromethyl-2H-chromene-3-carboxylic acid or derivative thereof, the method comprising:
(c) introducing a mixture of the enantiomers to a chiral stationary phase; and
(d) eluting at least one of the enantiomers with a mobile phase;
wherein the substituted 2-trifluoromethyl-2H-chromene-3-carboxylic acid or derivative thereof is a compound of Formulas I″, I′, I, or II, wherein the compound of Formulas I″, I′, I, or II is as defined above. Another aspect of this invention relates to a method for separating a mixture of enantiomers of a substituted 2-trifluoromethyl-2H-chromene-3-carboxylic acid or a derivative thereof, the method comprising subjecting the mixture to enantioselective fractional crystallization, enantioselective high performance liquid chromatography (“HPLC”), enantioselective steady state recycling chromatography (“SSRC”), or enantioselective multicolumn chromatography (“MCC”). Additional aspects of the present invention are described above and below.

A derivative of a substituted 2-trifluoromethyl-2H-chromene-3-carboxylic acid includes a substituted 2-trifluoromethyl-2H-chromene-3-carboxylic ester, a substituted 2-trifluoromethyl-1,2-dihydro-quinoline-3-carboxylic acid and ester, a substituted 2-trifluoromethyl-2H-thiochromene-3-carboxylic acid and ester, and a substituted 3-trifluoromethyl-3,4-dihydro-naphthalene-2-carboxylic acid and ester, and a pharmaceutically acceptable salt thereof.

An “acid derivative” of a substituted 2-trifluoromethyl-2H-chromene-3-carboxylic acid includes a substituted 2-trifluoromethyl-1,2-dihydro-quinoline-3-carboxylic acid, a substituted 2-trifluoromethyl-2H-thiochromene-3-carboxylic acid, and a substituted 3-trifluoromethyl-3,4-dihydro-naphthalene-2-carboxylic acid.

An “ester derivative” of a substituted 2-trifluoromethyl-2H-chromene-3-carboxylic acid includes a substituted 2-trifluoromethyl-2H-chromene-3-carboxylic ester, a substituted 2-trifluoromethyl-1,2-dihydro-quinoline-3-carboxylic ester, a substituted 2-trifluoromethyl-2H-thiochromene-3-carboxylic ester, and a substituted 3-trifluoromethyl-3,4-dihydro-naphthalene-2-carboxylic ester.

A pharmaceutically acceptable salt derivative of a substituted 2-trifluoromethyl-2H-chromene-3-carboxylic acid includes a pharmaceutically acceptable salt of a substituted 2-trifluoromethyl-2H-chromene-3-carboxylic ester, a pharmaceutically acceptable salt of a substituted 2-trifluoromethyl-1,2-dihydro-quinoline-3-carboxylic acid and ester, a pharmaceutically acceptable salt of a substituted 2-trifluoromethyl-2H-thiochromene-3-carboxylic acid and ester, and a pharmaceutically acceptable salt of a substituted 3-trifluoromethyl-3,4-dihydro-naphthalene-2-carboxylic acid and ester.

For purposes herein, a substituted 2-trifluoromethyl-2H-chromene-3-carboxylic acid or ester, or a pharmaceutically acceptable salt thereof (i.e., a compound of Formulas I″, I′, I, or II wherein X is O), a substituted 2-trifluoromethyl-1,2-dihydro-quinoline-3-carboxylic acid or ester, or a pharmaceutically acceptable salt thereof (i.e., a compound of Formulas I″, I′, or I, wherein X is NR^{a} or a compound of Formula II wherein is NH), and a substituted 2-trifluoromethyl-2H-thiochromene-3-carboxylic acid or ester, or a pharmaceutically acceptable salt thereof (i.e., a compound of Formulas I″, I′, I, or II wherein X is S), will have the ring numbering scheme illustrated below: 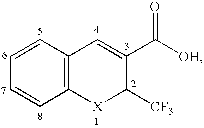
wherein X is O, S, NH, or NR^{a}.

For purposes herein, a substituted 3-trifluoromethyl-3,4-dihydro-naphthalene-2-carboxylic acid or ester, or a pharmaceutically acceptable salt thereof (i.e., a compound of Formulas I″, I′, or I wherein X is CR^{c}R^{b}), will have the ring numbering scheme illustrated below: 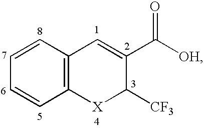
wherein X is CR^{c}R^{b}.

A 2H-chromene-3-carboxylic acid is also known as a 2H-1-benzopyran-3-carboxylic acid.

For a compound of Formulas I″, I′, and I, the following terms are defined:

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where the term “alkyl” is used, either alone or within other terms such as “haloalkyl” and “alkylsulfonyl”, it embraces linear or branched radicals having one to about twenty carbon atoms or, preferably, one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Even more preferred are lower alkyl radicals having one to three carbon atoms.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl” and “lower alkenyl”, embrace radicals having “cis” and “trans” orientations, or alternatively, “E” and “Z” orientations.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals.

“Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

“Perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “cyanoalkyl” embraces linear or branched alkyl radicals having one to about ten carbon atoms any one of which may be substituted with one cyano radicals. More preferred cyanoalkyl radicals are “lower cyanoalkyl” radicals having one to six carbon atoms and one cyano radical. Even more preferred are lower cyanoalkyl radicals having one to three carbon atoms. Examples of such radicals include cyanomethyl.

The terms “alkoxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. The “alkoxy” radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination in other terms (e.g., aryl-C₁-C₃ alkyl), means a carbocyclic aromatic system containing one or two rings wherein such rings may be attached together in a pendent manner or may be fused. The term “aryl” embraces aromatic radicals such as phenyl, naphthyl, tetrahydronaphthyl, indane and biphenyl. More preferred aryl is phenyl. The “aryl” group may have 1 to 3 substituents such as lower alkyl, hydroxy, halo, haloalkyl, nitro, cyano, alkoxy and lower alkylamino.

The term “heterocyclyl” embraces saturated, partially saturated and unsaturated heteroatom-containing ring-shaped radicals, where the heteroatoms may be selected from nitrogen, sulfur and oxygen. Examples of saturated heterocyclic radicals include saturated 3 to 6-membered heteromonocylic group containing 1 to 4 nitrogen atoms [e.g. pyrrolidinyl, imidazolidinyl, piperidino, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothiophene, dihydropyran, dihydrofuran and dihydrothiazole. Examples of unsaturated heterocyclic radicals, also termed “heteroaryl” radicals, include unsaturated 5 to 6 membered heteromonocyclyl group containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl]; unsaturated 3 to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated condensed heterocyclic group containing 1 to 2 oxygen atoms and 1 to 3 nitrogen atoms [e.g. benzoxazolyl, benzoxadiazolyl]; unsaturated to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl]; unsaturated condensed heterocyclic group containing 1 to 2 sulfur atoms and 1 to 3 nitrogen atoms [e.g., benzothiazolyl, benzothiadiazolyl] and the like. The term also embraces radicals where heterocyclic radicals are fused with aryl radicals. Examples of such fused bicyclic radicals include benzofuran, benzothiophene, and the like. The “heterocyclyl” group may have 1 to 3 substituents such as lower alkyl, hydroxy, oxo, amino and lower alkylamino. Preferred heterocyclic radicals include five to ten membered fused or unfused radicals. More preferred examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, oxazolyl, furyl, and pyrazinyl. Even more preferred heteroaryl radicals are 5- or 6-membered heteroaryl, containing one or two heteroatoms selected from sulfur nitrogen and oxygen, selected from thienyl, furanyl, pyrrolyl, thiazolyl, oxazolyl, imidazolyl, pyrazolyl, isoxazolyl, isothiazolyl, pyridyl, piperidinyl and pyrazinyl.

The term “sulfonyl”, whether used alone or linked to other terms such as alkylsulfonyl, denotes respectively divalent radicals —SO₂—.

“Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Even more preferred are lower alkylsulfonyl radicals having one to three carbon atoms. Examples of such lower alkylsulfonyl radicals include methylsulfonyl, ethylsulfonyl and propylsulfonyl.

“Haloalkylsulfonyl” embraces haloalkyl radicals attached to a sulfonyl radical, where haloalkyl is defined as above. More preferred haloalkylsulfonyl radicals are “lower haloalkylsulfonyl” radicals having one to six carbon atoms. Even more preferred are lower haloalkylsulfonyl radicals having one to three carbon atoms. Examples of such lower haloalkylsulfonyl radicals include trifluoromethylsulfonyl.

The term “arylalkylsulfonyl” embraces aryl radicals as defined above, attached to an alkylsulfonyl radical. Examples of such radicals include benzylsulfonyl and phenylethylsulfonyl.

The term “heterocyclosulfonyl” embraces heterocyclo radicals as defined above, attached to a sulfonyl radical. More preferred heterocyclosulfonyl radicals contain 5-7 membered heterocyclo radicals containing one or two heteroatoms. Examples of such radicals include tetrahydropyrrolylsulfonyl morpholinylsulfonyl and azepinylsulfonyl.

The terms “sulfamyl,” “aminosulfonyl” and “sulfonamidyl,” whether alone or used with terms such as “N-alkylaminosulfonyl”, “N-arylaminosulfonyl”, “N,N-dialkylaminosulfonyl” and “N-alkyl-N-arylaminosulfonyl”, denotes a sulfonyl radical substituted with an amine radical, forming a sulfonamide (—SO₂NH₂).

The term “alkylaminosulfonyl” includes “N-alkylaminosulfonyl” and “N,N-dialkylaminosulfonyl” where sulfamyl radicals are substituted, respectively, with one alkyl radical, or two alkyl radicals. More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, N-ethylaminosulfonyl and N-methyl-N-ethylaminosulfonyl.

The terms “N-arylaminosulfonyl” and “N-alkyl-N-arylaminosulfonyl” denote sulfamyl radicals substituted, respectively, with one aryl radical, or one alkyl and one aryl radical. More preferred N-alkyl-N-arylaminosulfonyl radicals are “lower N-alkyl-N-arylsulfonyl” radicals having alkyl radicals of one to six carbon atoms. Even more preferred are lower N-alkyl-N-arylsulfonyl radicals having one to three carbon atoms. Examples of such lower N-alkyl-N-aryl-aminosulfonyl radicals include N-methyl-N-phenylaminosulfonyl and N-ethyl-N-phenylaminosulfonyl. Examples of such N-aryl-aminosulfonyl radicals include N-phenylaminosulfonyl.

The term “arylalkylaminosulfonyl” embraces aralkyl radicals as described above, attached to an aminosulfonyl radical. More preferred are lower arylalkylaminosulfonyl radicals having one to three carbon atoms.

The term “heterocyclylaminosulfonyl” embraces heterocyclyl radicals as described above, attached to an aminosulfonyl radical.

The terms “carboxy” or “carboxyl”, whether used alone or with other terms, such as “carboxyalkyl”, denotes —CO₂H.

The term “carboxyalkyl” embraces radicals having a carboxy radical as defined above, attached to an alkyl radical.

The term “carbonyl”, whether used alone or with other terms, such as “alkylcarbonyl”, denotes —(C═O)—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “aroyl” embraces aryl radicals with a carbonyl radical as defined above. Examples of aroyl include benzoyl, naphthoyl, and the like and the aryl in the aroyl may be additionally substituted.

The term “alkylcarbonyl” embraces radicals having a carbonyl radical substituted with an alkyl radical. More preferred alkylcarbonyl radicals are “lower alkylcarbonyl” radicals having one to six carbon atoms. Even more preferred are lower alkylcarbonyl radicals having one to three carbon atoms. Examples of such radicals include methylcarbonyl and ethylcarbonyl.

The term “haloalkylcarbonyl” embraces radicals having a carbonyl radical substituted with a haloalkyl radical. More preferred haloalkylcarbonyl radicals are “lower haloalkylcarbonyl” radicals having one to six carbon atoms. Even more preferred are lower haloalkylcarbonyl radicals having one to three carbon atoms. Examples of such radicals include trifluoromethylcarbonyl.

The term “arylcarbonyl” embraces radicals having a carbonyl radical substituted with an aryl radical. More preferred arylcarbonyl radicals include phenylcarbonyl.

The term “heteroarylcarbonyl” embraces radicals having a carbonyl radical substituted with a heteroaryl radical. Even more preferred are 5- or 6-membered heteroarylcarbonyl radicals.

The term “arylalkylcarbonyl” embraces radicals having a carbonyl radical substituted with an arylalkyl radical. More preferred radicals are phenyl-C₁-C₃-alkylcarbonyl, including benzylcarbonyl.

The term “heteroarylalkylcarbonyl” embraces radicals having a carbonyl radical substituted with a heteroarylalkyl radical. Even more preferred are lower heteroarylalkylcarbonyl radicals having 5-6-membered heteroaryl radicals attached to alkyl portions having one to three carbon atoms.

The term “alkoxycarbonyl” means a radical containing an alkoxy radical, as defined above, attached via an oxygen atom to a carbonyl radical. Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl Even more preferred are lower alkoxycarbonyl radicals having alkoxy portions of one to three carbon atoms.

The term “aminocarbonyl” when used by itself or with other terms such as “aminocarbonylalkyl”, “N-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂.

The terms “N-alkylaminocarbonyl” and “N,N-dialkylaminocarbonyl” denote aminocarbonyl radicals which have been substituted with one alkyl radical and with two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The term “N-cycloalkylaminocarbonyl” denotes aminocarbonyl radicals which have been substituted with at least one cycloalkyl radical. More preferred are “lower cycloalkylaminocarbonyl” having lower cycloalkyl radicals of three to seven carbon atoms, attached to an aminocarbonyl radical.

The term “aminoalkyl” embraces alkyl radicals substituted with amino radicals.

The term “alkylaminoalkyl” embraces aminoalkyl radicals having the nitrogen atom substituted with an alkyl radical. Even more preferred are lower alkylaminoalkyl radicals having one to three carbon atoms.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are lower aralkyl radicals phenyl attached to alkyl portions having one to three carbon atoms. Examples of such radicals include benzyl, diphenylmethyl and phenylethyl. The aryl in the aralkyl may be additionally substituted with halo, alkyl, alkoxy, haloalkyl and haloalkoxy.

The term “arylalkenyl” embraces aryl-substituted alkenyl radicals. Preferable arylalkenyl radicals are “lower arylalkenyl” radicals having aryl radicals attached to alkenyl radicals having two to six carbon atoms. Examples of such radicals include phenylethenyl. The aryl in the arylalkenyl may be additionally substituted with halo, alkyl, alkoxy, haloalkyl and haloalkoxy.

The term “arylalkynyl” embraces aryl-substituted alkynyl radicals. Preferable arylalkynyl radicals are “lower arylalkynyl” radicals having aryl radicals attached to alkynyl radicals having two to six carbon atoms. Examples of such radicals include phenylethynyl. The aryl in the aralkynyl may be additionally substituted with halo, alkyl, alkoxy, haloalkyl and haloalkoxy.

The terms benzyl and phenylmethyl are interchangeable.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃—S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylsulfinyl” embraces radicals containing a linear or branched alkyl radical, of one to ten carbon atoms, attached to a divalent —S(═O)— atom. More preferred are lower alkylsulfinyl radicals having one to three carbon atoms.

The term “arylsulfinyl” embraces radicals containing an aryl radical, attached to a divalent —S(═O)— atom. Even more preferred are optionally substituted phenylsulfinyl radicals.

The term “haloalkylsulfinyl” embraces radicals containing a haloalkyl radical, of one to ten carbon atoms, attached to a divalent —S(═O)— atom. Even more preferred are lower haloalkylsulfinyl radicals having one to three carbon atoms.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable “alkylamino” may be mono or dialkylamino such as N-methylamino, N-ethylamino, N,N-dimethylamino, N,N-diethylamino or the like.

The term “arylamino” denotes amino groups which have been substituted with one or two aryl radicals, such as N-phenylamino. The “arylamino” radicals may be further substituted on the aryl ring portion of the radical.

The term “heteroarylamino” denotes amino groups which have been substituted with one or two heteroaryl radicals, such as N-thienylamino. The “heteroarylamino” radicals may be further substituted on the heteroaryl ring portion of the radical.

The term “aralkylamino” denotes amino groups which have been substituted with one or two aralkyl radicals. More preferred are phenyl-C₁-C₃-alkylamino radicals, such as N-benzylamino. The “aralkylamino” radicals may be further substituted on the aryl ring portion of the radical.

The terms “N-alkyl-N-arylamino” and “N-aralkyl-N-alkylamino” denote amino groups which have been substituted with one aralkyl and one alkyl radical, or one aryl and one alkyl radical, respectively, to an amino group.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aralkylsulfonyl” embraces aralkyl radicals as described above, attached to a divalent sulfonyl radical. More preferred are phenyl-C₁-C₃-alkylsulfonyl radicals.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

For a compound of Formula II, groups R⁶ to R¹⁰, the following terms are defined:

“Alkyl”, “alkenyl,” and “alkynyl” unless otherwise noted are each straight chain or branched chain hydrocarbons of from one to twenty carbons for alkyl or two to twenty carbons for alkenyl and alkynyl in the present invention and therefore mean, for example, methyl, ethyl, propyl, butyl, pentyl or hexyl and ethenyl, propenyl, butenyl, pentenyl, or hexenyl and ethynyl, propynyl, butynyl, pentynyl, or hexynyl respectively and isomers thereof.

“Aryl” means a fully unsaturated mono- or multi-ring carbocycle, including, but not limited to, substituted or unsubstituted phenyl, naphthyl, or anthracenyl.

“Heterocycle” means a saturated or unsaturated mono- or multi-ring carbocycle wherein one or more carbon atoms can be replaced by N, S, P, or O. This includes, for example, the following structures: 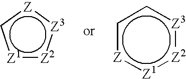
wherein Z, Z¹, Z² or Z³ is C, S, P, O, or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only if Z, Z¹, Z² or Z³ is C.

The term “heteroaryl” means a fully unsaturated heterocycle.

In either “heterocycle” or “heteroaryl,” the point of attachment to the molecule of interest can be at the heteroatom or elsewhere within the ring.

Illustrative examples of heterocycle and heteroaryl groups are provided above in the definition of terms used for Formulas I″, I′, and I.

The term “hydroxy” means a group having the structure —OH.

The term “halogen” or “halo” means a fluoro, chloro, bromo or iodo group.

The term “haloalkyl” means alkyl substituted with one or more halogens.

The term “cycloalkyl” means a mono- or multi-ringed carbocycle wherein each ring contains three to ten carbon atoms, and wherein any ring can contain one or more double or triple bonds. Examples include radicals such as cyclopropyl, cyclobutyl, cyclopentyl, cyclohexyl, cycloalkenyl, and cycloheptyl. The term “cycloalkyl” additionally encompasses spiro systems wherein the cycloalkyl ring has a carbon ring atom in common with the seven-membered heterocyclic ring of the benzothiepine.

The term “oxo” means a doubly bonded oxygen.

The term “cycloalkylidene” means a mono- or multi-ringed carbocycle wherein a carbon within the ring structure is doubly bonded to an atom which is not within the ring structures.

The term “nitro” means a group having the formula —NO₂.

The term “sulfo” means a sulfo group, —SO₃H, or its salts.

The term “thio” means a group having the formula —SH.

The term “sulfoalkyl” means an alkyl group to which a sulfonate group is bonded, wherein the alkyl is bonded to the molecule of interest.

The term “aminosulfonyl” means a group having the formula —SO₂NH₂.

The term “alkylthio” means a moiety containing an alkyl radical which is attached to an sulfur atom, such as a methylthio radical. The alkylthio moiety is bonded to the molecule of interest at the sulfur atom of the alkylthio.

The term “aryloxy” a moiety containing an aryl radical which is attached to an oxygen atom, such as a phenoxy radical. The aryloxy moiety is bonded to the molecule of interest at the oxygen atom of the aryloxy.

The term “alkenyloxy” a moiety containing an alkenyl radical which is attached to an oxygen atom, such as a 3-propenyloxy radical. The alkenyloxy moiety is bonded to the molecule of interest at the oxygen atom of the alkenyloxy.

The term “arylalkyl” means an aryl-substituted alkyl radical such as benzyl. The term “alkylarylalkyl” means an arylalkyl radical that is substituted on the aryl group with one or more alkyl groups.

The term “amino” means a group having the structure —NH₂. Optionally the amino group can be substituted for example with one, two or three groups such as alkyl, alkenyl, alkynyl, aryl, and the like.

The tern “cyano” means a group having the structure —CN.

The term “heterocyclylalkyl” means an alkyl radical that is substituted with one or more heterocycle groups.

The term “heteroarylalkyl” means an alkyl radical that is substituted with one or more heteroaryl groups.

The term “alkylheteroarylalkyl” means a heteroarylalkyl radical that is substituted with one or more alkyl groups.

The term “alkoxy” means a moiety containing an alkyl radical which is attached to an oxygen atom, such as a methoxy radical. The alkoxy moiety is bonded to the molecule of interest at the oxygen atom of the alkoxy. Examples of such radicals include methoxy, ethoxy, propoxy, iso-propoxy, butoxy and tert-butoxy.

The term “carboxy” means the carboxy group, —CO₂H, or its salts.

The term “carbonyl” means a carbon atom doubly bonded to an oxygen atom.

The term “carboxyalkyl” means an alkyl radical that is substituted with one or more carboxy groups. Preferable carboxyalkyl radicals are “lower carboxyalkyl” radicals having one or more carboxy groups attached to an alkyl radical having one to six carbon atoms.

The term “carboxyheterocycle” means a heterocycle radical that is substituted with one or more carboxy groups.

The term “carboxyheteroaryl” means a heteroaryl radical that is substituted with one or more carboxy groups.

The term “carboalkoxyalkyl” means an alkyl radical that is substituted with one or more alkoxycarbonyl groups. Preferable carboalkoxyalkyl radicals are “lower carboalkoxyalkyl” radicals having one or more alkoxycarbonyl groups attached to an alkyl radical having one to six carbon atoms.

The term “carboxyalkylamino” means an amino radical that is mono- or di-substituted with carboxyalkyl. Preferably, the carboxyalkyl substituent is a “lower carboxyalkyl” radical wherein the carboxy group is attached to an alkyl radical having one to six carbon atoms.

When used in terms that contain a combination of terms, for example “alkylaryl” or “arylalkyl,” the individual terms (e.g., alkyl, aryl) listed above have the meaning indicated above.

The compounds of Formulas I″, I′, I, and II, and the pharmaceutically acceptable salts thereof, are selective COX-2 inhibitors, which means that they are selective inhibitors of the COX-2 over COX-1. Preferably, the compounds of Formulas I″, I′, I, and II, and the pharmaceutically acceptable salts thereof, when assayed with COX-2 have IC₅₀ values of less than about 0.5 μM, and also have selectivity ratios of COX-2 inhibition over COX-1 inhibition of at least 50, and more preferably of at least 100. The COX-2 and COX-1 inhibitory activity is determined according to biological method “b. Assay for COX-1 and COX-2 Activity” of U.S. Pat. No. 6,077,850, column 169, beginning at line 15. The selectivity ratio is the IC₅₀ determined with COX-1 divided by the IC₅₀ ratio determined with COX-2, wherein each IC₅₀ is the concentration of a compound of Formulas I″, I′, I, or II, or a the pharmaceutically acceptable salt thereof, in micromolar that is needed to inhibit the enzyme being assayed by 50%.

The compounds of Formulas I″, I′, I, and II, and the pharmaceutically acceptable salts thereof, may be formulated for pharmaceutical use and administered to a mammal, including a human, to treat diseases such as arthritis and pain as described in U.S. Pat. No. 6,034,256; 6,077,850; 6,218,427; or 6,271,253 or U.S. patent application Ser. No. 10/801,446 or 10/801,429.

Many substituted 2-trifluoromethyl-2H-chromene-3-carboxylic acids, and esters having a basic nitrogen atom, are capable of further forming pharmaceutically acceptable salts, including, but not limited to, base addition salts and acid addition salts, respectively.

Another aspect of this invention is a method for separating enantiomers of a pharmaceutically acceptable salt of a substituted 2-trifluoromethyl-2H-chromene-3-carboxylic acid or derivative thereof, the method comprising:
(a) introducing a mixture of the enantiomers to a reverse phase, chiral stationary phase; and
(b) eluting at least one of the enantiomers with a mobile phase;
wherein the substituted 2-trifluoromethyl-2H-chromene-3-carboxylic acid or derivative thereof is a compound of Formulas I″, I′, I, or II 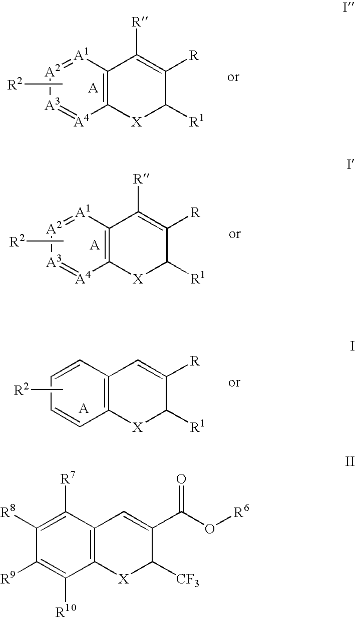
wherein:
for Formula I″, R¹, R, R″, R², A, A¹, A², A³, A⁴, and X are as they are defined above for Formula I″;
for Formula I′, R¹, R, R″, R², A, A¹, A², A³, A⁴, and X are as they are defined for Formula I′;
for Formula I, R¹, R, R″, R², A, A¹, A², A³, A⁴, and X are as they are defined for Formula I;
For Formula II, X, R⁶, R⁷, R⁸, R⁹, and R¹⁰ are as they are defined above for Formula II.

Another aspect of this invention is any one of the above or below methods for separating enantiomers of a substituted 2-trifluoromethyl-2H-chromene-3-carboxylic acid or derivative thereof, the method further comprising a step of subjecting at least one of the enantiomers in the eluate produced in the eluting step to interconverting with its antipode by irradiation with ultraviolet (“UV”) or visible light to produce a mixture of the at least one enantiomer and its antipode in the eluate.

Another aspect of this invention is the above method for separating enantiomers of a substituted 2-trifluoromethyl-2H-chromene-3-carboxylic acid or derivative thereof, the method further comprising a step of subjecting the mixture of the at least one enantiomer and its antipode in the eluate to an enantioselective steady state recycling chromatography or an enantioselective multicolumn chromatography.

Another aspect of this invention is any one of the above or below methods for separating enantiomers of a pharmaceutically acceptable salt of the substituted 2-trifluoromethyl-2H-chromene-3-carboxylic acid or derivative thereof, the method further comprising a step of monitoring the eluate produced in the eluting step for at least one of the enantiomers.

Another aspect of this invention is any one of the above or below methods for separating enantiomers of a pharmaceutically acceptable salt of the substituted 2-trifluoromethyl-2H-chromene-3-carboxylic acid or derivative thereof, wherein the mobile phase is:
a buffered neutral aqueous solution and a polar solvent;
a buffered acidic aqueous solution and a polar solvent; or a buffered basic aqueous solution and a polar solvent,
   wherein the polar solvent comprises from about 5% to about 95% volume/volume of the mobile phase.

Another aspect of this invention is any one of the above or below methods for separating enantiomers of a pharmaceutically acceptable salt of the substituted 2-trifluoromethyl-2H-chromene-3-carboxylic acid or derivative thereof, wherein the enantiomers are:
A pharmaceutically acceptable salt of (R)- and (S)-6-chloro-7-tert-butyl-2-trifluoromethyl-2H-chromene-3-carboxylic acid;
A pharmaceutically acceptable salt of (R)- and (S)-6-chloro-8-methyl-2-trifluoromethyl-2H-chromene-3-carboxylic acid;
A pharmaceutically acceptable salt of (R)- and (S)-6,8-dimethyl-2-trifluoromethyl-2H-chromene-3-carboxylic acid; or
A pharmaceutically acceptable salt of (R)- and (S)-8-ethyl-6-trifluoromethoxy-2-trifluoromethyl-2H-chromene-3-carboxylic acid.

Another aspect of this invention is any one of the above or below methods for separating enantiomers of a pharmaceutically acceptable salt of the substituted 2-trifluoromethyl-2H-chromene-3-carboxylic acid or derivative thereof, wherein the enantiomers are:
A pharmaceutically acceptable salt of (R)- and (S)-8-chloro-6-methoxy-2-trifluoromethyl-2H-chromene-3-carboxylic acid;
A pharmaceutically acceptable salt of (R)- and (S)-6-chloro-7-(1,1-dimethyl-2-hydroxyethyl)-2-trifluoromethyl-2H-chromene-3-carboxylic acid;
A pharmaceutically acceptable salt of (R)- and (S)-6-chloro-7-benzyl-2-trifluoromethyl-2H-chromene-3-carboxylic acid;
A pharmaceutically acceptable salt of (R)- and (S)-6-chloro-5,7-dimethyl-2-trifluoromethyl-2H-chromene-3-carboxylic acid;
A pharmaceutically acceptable salt of (R)- and (S)-6-ethyl-8-methyl-2-trifluoromethyl-2H-chromene-3-carboxylic acid; or
A pharmaceutically acceptable salt of (R)- and (S)-6-chloro-5-methyl-2-trifluoromethyl-2H-chromene-3-carboxylic acid.

Another aspect of this invention is any one of the above or below methods for separating enantiomers of a pharmaceutically acceptable salt of the substituted 2-trifluoromethyl-2H-chromene-3-carboxylic acid or derivative thereof, the method further comprising a step of subjecting at least one of the enantiomers in the eluate produced in the eluting step to irradiating using a high-intensity ultraviolet light source to produce a mixture of the at least one enantiomer and its antipode in the eluate. The reaction mixture may further comprise an UV sensitive, photo-converting-promoting additive.

The phrase “irradiating using a high-intensity ultraviolet light source” means directing an electrical ultraviolet (“UV”) light source at the object being irradiated, wherein the intensity of the UV light source is at least about 0.1-Watts per square centimeter (“W/cm²”), preferably at least about 0.2-W/cm², or is of sufficient intensity to produce a photoracemized mixture of enantiomers having an enantiomeric excess that is less than 90% of the e.e. of the starting enantiomer within a 24 hour period or is of sufficient intensity to result in a half-life of the enantiomer being irradiated of 24 hours or less. For illustration, a 450-W UV light source shining through a glass cylinder (e.g., quartz) having a 25 cm length and a diameter of 8 cm would have an intensity of 450-W÷(25 cm×8 cmπ)=0.72-W/cm². The rate of photoracemization is proportional to the intensity of UV light from each high-intensity UV light source being used and to the number of UV light sources being used, and inversely proportional to the distance between the UV light source and the enantiomer.

The high intensity UV light source includes a UV spot lamp, a UV photoreactor, or a UV photoreactor flow through cell. A total of 1, 2, 4, 6, 12, 20, 50, 100, 200 or more high intensity UV light sources may be used. When a UV photoreactor flow through cell is used in the invention method, the percent decrease of e.e. is inversely proportional to the flow rate of the mixture being passed through the cell. A total of 1, 2, 4, 6, 12, or more flow through photoreactor cells may be used.

High intensity UV light sources are readily available from commercial sources and for purposes of practicing the photoracemization method of the present invention it does not matter which particular type or brand of UV light source is used.

UV light is a spectrum of light having a wavelength of from about 210 nm to about 450 nm. UV-absorbing materials such as a UV-absorbing chiral auxiliary or a UV-absorbing solvent may be present during the method of photo-converting step provided that they do not absorb the particular wavelength(s) of UV light being used for irradiation to the extent described above.

Another aspect of this invention is any one of the above or below methods for separating enantiomers of a pharmaceutically acceptable salt of the substituted 2-trifluoromethyl-2H-chromene-3-carboxylic acid or derivative thereof, the method further comprising a step of subjecting the mixture of the at least one enantiomer and its antipode in the eluate to an enantioselective multicolumn chromatography

Another aspect of this invention is any one of the above or below methods for separating enantiomers of a pharmaceutically acceptable salt of the substituted 2-trifluoromethyl-2H-chromene-3-carboxylic acid or derivative thereof, the method further comprising a subsequent step of subjecting at least one of the separated enantiomers to enantioselective fractional crystallization.

The terms “pharmaceutically-acceptable salts” and “pharmaceutically acceptable salts” are synonymous. Both terms embrace salts commonly used to form alkali metal salts and to form addition salts of free acids or free bases. The nature of the salt is not critical, provided that it is pharmaceutically acceptable. Suitable pharmaceutically-acceptable acid addition salts of compounds of Formulas I″, I′, I, and II may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicyclic, galactaric and galacturonic acid. Suitable pharmaceutically-acceptable base addition salts of compounds of Formulas I″, I′, I, and II include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of Formulas I″, I′, I, and II.

The phrase “mixture of the enantiomers” includes racemic and non-racemic mixtures. The mixture of the enantiomers is typically introduced to the chiral stationary phase as a solution. Preferably, the solution comprises mobile phase or a component or components thereof.

The phrase “separated enantiomers” includes all non-racemic mixtures of the enantiomers that are obtained from the separation of a racemic mixture of the enantiomers and all non-racemic mixtures of the enantiomers wherein the enantiomeric purity of at least one of the enantiomers is increased by 1%, 2%, 4%, or 5% compared to the enantiomeric purity of the enantiomer before separation.

Mobile phase may comprise a single solvent or a soluble mixture of 2, 3, 4, 5, 6, 7, or more solvents.

Mobile phase may also comprise at least one additive. An additive suitable for chromatography of the acid or ester on a chiral stationary phase is typically an amine such as trimethylamine, triethylamine, and the like or an organic salt such as sodium or potassium acetate or an inorganic salt such as ammonium acetate or ammonium chloride. An additive suitable for chromatography of the salt of the acid or ester on a reverse phase, chiral stationary phase is typically an inorganic salt such as those described herein.

The phrase “supercritical fluid” means a liquefied carbon dioxide.

Eluate may or may not contain a substituted 2-trifluoromethyl-2H-chromene-3-carboxylic acid or derivative thereof dissolved therein. Eluate may be collected for analysis of any material dissolved therein or for isolation and recovery of any material dissolved therein by conventional means such as by evaporation of mobile phase, optionally with crystallization of the material. Alternatively, eluate may be recycled directly by reintroduction to the stationary phase via a recycle stream or indirectly by introduction to an interconverting unit followed by introduction of the resulting mixture of the at least one enantiomer and its antipode to the stationary phase via a recycle stream. Still alternatively, eluate may be introduced to another stationary phase (chiral or achiral), which may be the same or different than the previous stationary phase and is flowingly connected to the prior stationary phase. In enantioselective MCC, eluate includes a raffinate stream, wherein the mobile phase contains dissolved therein a majority of one enantiomer of the acid, ester, or salt thereof, and an extract stream, wherein the mobile phase contains dissolved therein a majority of the other enantiomer of the acid, ester, or salt thereof. Eluate streams may or may not contain one or both of the enantiomers dissolved therein.

Eluate can be monitored for the presence or absence of at least one enantiomer of the substituted 2-trifluoromethyl-2H-chromene-3-carboxylic acid or derivative thereof by any conventional means such as, for example, by passing the eluate, or a portion thereof, through a detector. The detector may be compatible with liquid chromatography or not and may be capable of determining chirality or not. Illustrative examples of detectors compatible with liquid chromatography include ultraviolet detectors, photodiode array detectors that may scan ultraviolet light wavelengths from about 210 nm wavelength to about 320 nm wavelength (e.g., 210 nm, 240 nm, 254 nm, 280 nm, or 290 nm) to detect UV-active components, devices that monitor rotation of plane polarized light such as the IBZ CHIRALYSER available from JM Science, Inc., Grand Island, N.Y., refractive index detectors, and evaporative light scattering detectors. Alternatively, eluate may be monitored by timing fractions (e.g., when the retention time of an enantiomer is known); by sampling untimed or timed fractions and analyzing the samples by, for example, visual inspection, UV light illumination in conjunction with visual inspection, non-enantioselective or enantioselective HPLC, nuclear magnetic resonance, mass spectrometry, derivatization and analysis of the resulting derivative, and the like; by evaporating fractions and analyzing the resulting residue for the presence of an enantiomer such as by visual inspection, UV light illumination in conjunction with visual inspection, melting point, non-enantioselective or enantioselective HPLC, nuclear magnetic resonance spectrometry, mass spectrometry, and the like; or by adding a derivatizing agent to fractions of the eluate or to the residue therefrom, and analyzing the resulting derivative as described above. Any method of monitoring that may be used to determine the presence of an enantiomer of the acids, esters, or pharmaceutically acceptable salts thereof, even if the method of monitoring cannot determine optical characteristics (i.e., the optical purity or e.e. of an enantiomer) of the enantiomer or whether the enantiomer is present with its antipode or not, is useful for monitoring the eluate.

Monitoring can be done simultaneously with an invention introducing or eluting step, after an invention introducing or eluting step, or both simultaneously with an invention introducing or eluting step and after the invention introducing or eluting step. For example in an enantioselective SMB chromatography, monitoring may not be done until after the introducing and eluting steps have been completed and after at least one of the enantiomers in the eluate has been isolated. Monitoring is any process or activity by which one of ordinary skill in the art would know whether any portion of eluate will contain, contains, or did contain at least one of the enantiomers.

The phrase “% volume/volume” equals (the volume of the liquid component in question divided by the volume of the mixture containing the component) times 100.

The phrase “% weight/weight” equals (the weight of the component in question divided by the weight of the mixture containing the component) times 100.

The phrase “enantioselective fractional crystallization” includes any crystallization that enriches by at least 1%, 2%, 4%, or 5% the enantiomeric purity of at least one enantiomer of a mixture of enantiomers, wherein the one enantiomer is optionally in the crystal phase or in the mother liquor therefrom. Enantioselective fractional crystallizations include crystallizations without a chiral auxiliary and co-crystallizations with a chiral auxiliary. Enantioselective fractional crystallizations include a crystallization of the major or minor enantiomer from a non-racemic mixture of major component and minor component enantiomers, wherein the crystals are enriched in the major component enantiomer and the mother liquor therefrom is enriched in the minor component enantiomer. Enantioselective fractional crystallizations also include a crystallization of a racemic mixture of enantiomers from a non-racemic mixture of enantiomers wherein the minor component enantiomer is enriched in the crystal phase and the major component enantiomer is enriched in the mother liquor therefrom. Enantioselective fractional crystallizations also include a co-crystallization of an enantiomer with a chiral auxiliary from a mixture (racemic or non-racemic) of enantiomers, wherein the crystals are enriched in one of the enantiomers and the mother liquor therefrom is enriched in the other enantiomer.

The phrase “chiral auxiliary” means a chiral organic amine that is capable of forming a crystalline salt with a substituted 2-trifluoromethyl-2H-chromene-3-carboxylic acid or acid derivative thereof or a chiral organic acid that is capable of forming a crystalline salt with a basic substituted 2-trifluoromethyl-2H-chromene-3-carboxylic ester or ester derivative thereof. A chiral organic amine auxiliary that is useful in a method of the present invention may be selected from the group consisting of: L-tert-Leucinol, (+)-Cinchonine, Quinine, (1R,2S)-(+)-cis-1-Amino-2-indanol, (DHQ)₂ PHAL, L-Proline, L-Phenyl glycine methyl ester, (R)-N-Benzyl-1-(1-naphthy)ethylamine, Tetramisole HCl, (1S,2S)-(+)-Thiomicamine, R-(+)-4-Diphenylmethyl-2-oxazolidinone, R-(+)-N,N-Dimethyl-1-phenylethylamine, L-Valinol, (1R,2R)-(−)-1,2-Diaminocyclohexane, (1R,2S)-2-Amino-1,2-diphenylethanol, (+)-Bis [(R)-1-phenylethyl] Amine, L-Prolinol, (S)-(−)-α-Methyl-benzylamine, (1S,2S)-(+)-2-Amino-1-phenyl-1,3-propanediol, (1R,2S)-(−)-Ephedrine, L-Phenylalanine ethyl ester, L-Phenylalaninol, (R)-(−)-3-methyl-2-butylamine, (1R,2R)-(+)-1,2-Diphenyl ethylenediamine, (1S,2R)-(+)-Norephedrine, (R)-(+)-N-Benzyl-α-Methylbenzylamine, (+)-(2S,3R)-4-Dimethyl amino-3-methyl-1,2-diphenyl-2-butanol, R-(+)-1-(1-Naphthyl)ethylamine, R-(+)-1-(4-Bromophenyl)ethylamine, (−)-Cinchonidine, D-Glucamine, (S)-(−)-1-Benzyl-2-pyrrolidinemethanol, (1R,2S)-(−)-N-Methylephedrine, Quinidine, (R)-(−)-2-Phenylglycinol, R-(−)-1-(4-Nitrophenyl)ethylamine, R-(−)-2-Amino-1-butanol, (R)-(−)-1-Cyclohexylethylamine, N-Methyl-D-glucamine, (8S,9R)-(−)-N-Benzylcinchoninium chloride, 1-Deoxy-1-(methylamino)-D-galactitol, (1R,2S)-(+)-cis-[-2-(Benzylamine)cyclohexyl]methanol, (1R,2R)-(−)-2-Amino-1-(4-nitrophenyl)-1,3-propanediol, L-Phenylalanine methyl ester, (1S,2S)-(+)-Pseudoephedrine, and (S)-1-methoxy-2-propylamine.

A chiral organic amine auxiliary that is useful in a method of the present invention may also be selected from the group consisting of: (R)-(−)-1-Amino-2-propanol, (−)-cis-Myrtanylamine, (R)-1-(4-Methylphenyl)ethylamine, (S)-Aminotetraline, (R)-(−)-sec-butylamine, (R)-(−)-Tetrahydrofurfurylamine, (R)-3,3-dimethyl-2-butylamine, (R)-(−)-2-Aminoheptane, L-(+)-Isoleucinol, L-Leucinol, (R)-(−)-aminoindan, H-Methioninol, (S)-(−)-N,alpha-dimethyl-benzylamine, (S)-(−)-1-Phenylpropylamine, S-(−)-3-Tert-butylamino-1,2-propanediol, (R)-1-Methyl-3-phenylpropylamine, (R)-3-Amino-3-phenylpropan-1-ol, (R)-1-(3-methoxyphenyl)ethylamine, (R)-(+)-1(4-Methoxyphenyl)ethylamine, Methyl (R)-(+)-3-methyl glutarate, (S)-(−)-1-(2-Napthyl)ethylamine, L-tyrosinamide, S-Benzyl-L-cysteinol, (S)-1-phenyl-2-(p-tolyl)ethylamine, [R-(R*,R*)]-(+)-bis alpha-methylbenzylamine, (R)-(−)-N benzyl-2-phenylglycinol, L-tyrosinol, (R)-(+)-(3,4-dimethoxy)benzyl-1-phenylethylamine, and 1-deoxy-1-(octylamino)-D-glucitol.

A chiral organic amine auxiliary that is useful in a method of the present invention may also be selected from the group consisting of: (S)-(−)-α-methylbenzylamine, (−)-cinchonidine, (S)-(−)-2-amino-3-phenyl-1-propanol, (1R, 2S)-2-amino-1,2-diphenyl ethanol, (R)-(+)-4-diphenylmethyl-2-oxozolidinone, (1R,2S)-(+)-cis-[2-(benzylamine)cyclohexyl]methanol, (+)-quinine, (+)-cinchonine, L-phenylalaninol, (R)-(−)-2-amino-1-butanol, (R)-(−)-phenylglycinol, (1R,2R)-(+)-1,2-diphenylethylenediamine, (1S,2R)-(+)-norephedrine, (1R,2S)-(−)-N-methylephedrine, (1R,2S)-(−)-ephedrine, (+)-quinidine, (1R,2S)-(+)-1-amino-2-indanol, (1R,2R)-(−)-2-amino-1-(4-nitrophenyl)-1,3-propanediol, (R)-(+)-N-benzyl-α-methylbenzylamine, (+)-dehydroabietylamine, (+)-amphetamine, (+)-deoxyphedrine, (+)-chloramphenicol intermediate, (+)-1-(1-napthyl)ethylamine, (R)-(+)-α-methylbenzylamine, (+)-cinchonidine, (R)-(+)-2-amino-3-phenyl-1-propanol, (1S,2R)-2-amino-1,2-diphenyl ethanol, (S)-(−)-4-diphenylmethyl-2-oxozolidinone, (1S,2R)-(−)-cis-[2-(benzylamine)cyclohexyl]methanol, (−)-quinine, (−)-cinchonine, D-phenylalaninol, (S)-(+)-2-amino-1-butanol, (S)-(+)-phenylglycinol, (1S,2S)-(−)-1,2-diphenylethylenediamine, (1R,2S)-(−)-norephedrine, (1S,2R)-(+)-N-methylephedrine, (1S,2R)-(+)-ephedrine, (−)-quinidine, (1S,2R)-(−)-1-amino-2-indanol, (1S,2S)-(+)-2-amino-1-(4-nitrophenyl)-1,3-propanediol, (S)-(−)-N-benzyl-α-methylbenzylamine, (−)-dehydroabietylamine, (−)-amphetamine, (−)-deoxyphedrine, (−)-chloramphenicol intermediate, and (−)-1-(1-napthyl)ethylamine.

The phrase “substantially free of UV or visible light” means the absence of UV or visible light or a degree of exposure to ultraviolet or visible light that does not induce interconversion of (2R)- and (2S)-enantiomers (or (3R)- and (3S)-enantiomers in the case of 3,4-dihydro-naphthalene derivatives) of a substituted 2-trifluoromethyl-2H-chromene-3-carboxylic acid or derivative thereof or that does induce interconversion of less than 5% of the enantiomers during the course of practicing a method of the present invention.

The phrase “substantially free of mobile phase” means the absence of mobile phase or any component thereof, or the presence of less than 0.5% weight/weight of each component thereof.

The term “solution” means a mixture of the enantiomers that consists essentially of a solution, meaning there may be trace amounts of undissolved material (the enantiomers or impurities) that will not interfere with a successful practice of a method of this invention, typically because they can be filtered out prior to chromatography. A mobile phase useful in the method of the present invention is a substantially homogeneous solution at the proportions of individual components being used. It is expected that the individual components of the mobile phase will be miscible in any ratio specified by the method of the present invention without separation of any phases. A preferred solution is one wherein the solvent mixture comprises the mobile phase. Also preferred are solutions wherein the solvent comprises a solvent or mixture of two solvents that is a component of the mobile phase.

The phrase “feed solution” means a solution of the mixture of the enantiomers that is freshly introduced to the chiral stationary phase or reverse phase, chiral stationary phase.

The phrase “recycle solution” means eluate (e.g., raffinate or extract) containing a mixture of the enantiomers that is being reintroduced to the chiral stationary phase or reverse phase, chiral stationary phase. The enantiomers may optionally be first subjected to an interconverting step before being reintroduced to the chiral stationary phase or reverse phase, chiral stationary phase. A recycle solution embraces a solution recirculated internally to a chromatography pathway and an externally prepared solution that allows recycling of enantiomers that have been previously subject to a chromatography. In circumstances wherein an eluate stream (e.g., raffinate stream or extract stream) contains a less preferred enantiomer at a concentration that is less than optimum for productive separation of enantiomers, one aspect of the present invention is to recycle the eluate stream through an interconverting step and then dissolve in the irradiated stream an amount of the mixture of enantiomers (i.e., a mixture that has not yet been introduced to an instant chiral stationary phase or reverse phase, chiral stationary phase) to give a feed stream concentration that is about optimum for a productive separation of enantiomers.

The (2S)-enantiomer of a substituted 2-trifluoromethyl-2H-chromene-3-carboxylic acid or derivative thereof is the antipode of the corresponding (2R)-enantiomer of the substituted 2-trifluoromethyl-2H-chromene-3-carboxylic acid or derivative thereof. The (2R)-enantiomer of a substituted 2-trifluoromethyl-2H-chromene-3-carboxylic acid or derivative thereof is the antipode of the corresponding (2S)-enantiomer of the substituted 2-trifluoromethyl-2H-chromene-3-carboxylic acid or derivative thereof. The (3S)-enantiomer of a substituted 2-trifluoromethyl-2H-chromene-3-carboxylic acid or derivative thereof is the antipode of the corresponding (3R)-enantiomer of the substituted 2-trifluoromethyl-2H-chromene-3-carboxylic acid or derivative thereof. The (3R)-enantiomer of a substituted 2-trifluoromethyl-2H-chromene-3-carboxylic acid or derivative thereof is the antipode of the corresponding (3S)-enantiomer of the substituted 2-trifluoromethyl-2H-chromene-3-carboxylic acid or derivative thereof.

The term “interconverting” means a process, typically an equilibrium process, of inverting stereochemistry at the (2R)- and (2S)-(or (3R)- and (3S)-) chiral carbon atom in a substituted 2-trifluoromethyl-2H-chromene-3-carboxylic acid or derivative thereof. Interconverting includes, for example, subjecting the chiral compound (e.g., as a solid or dissolved in a solvent) to irradiation by light, for example irradiation by UV or visible light, wherein the chiral carbon atom comprises a photo-labile functional group, subjecting the chiral compound to base-catalyzed deprotonation followed by reprotonation, wherein the chiral carbon atom is bonded to an acidic hydrogen atom, and subjecting the chiral compound to a nucleophile-catalyzed bond cleavage followed by reforming of the broken bond and leaving of the nucleophile. Interconversion of (2R)- and (2S)-enantiomers (or (3R)- and (3S)-enantiomers) may or may not produce a racemic mixture of the chiral compound, depending upon the particular conditions used (e.g., method, reaction time, temperature, etc.). Interconversions include static and dynamic processes.

A static interconversion of (2R)- and (2S)-enantiomers (or (3R)- and (3S)-enantiomers) is an equilibrium process that would ultimately produce a racemic mixture if the process were conducted for a sufficient period of time. An illustrative example of a static interconversion is a light-promoted interconversion of a non-racemic mixture of the (2R)- and (2S)-enantiomers (or (3R)- and (3S)-enantiomers) of the substituted 2-trifluoromethyl-2H-chromene-3-carboxylic acid or derivative thereof to give a racemic mixture or a new non-racemic mixture having a lower enantiomeric excess.

A dynamic interconversion of (2R)- and (2S)-enantiomers (or (3R)- and (3S)-enantiomers) is a process that facilitates formation of one enantiomer over its antipode. Typically a dynamic interconversion is a process that has at least two steps in equilibrium or a process that has at least one step, wherein at least one of the steps is a non-equilibrium step. Illustrative examples of these two types of dynamic interconversions include a light-promoted interconversion of a less preferred enantiomer of the substituted 2-trifluoromethyl-2H-chromene-3-carboxylic acid or derivative thereof in the presence of a chiral auxiliary and precipitation or crystallization of the preferred enantiomer so formed as a salt with the chiral auxiliary, wherein the equilibrium favors the precipitated or crystallized salt over the solution of the salt, and a light-promoted interconversion of a less preferred enantiomer of the substituted 2-trifluoromethyl-2H-chromene-3-carboxylic acid or derivative thereof in the presence of a chiral auxiliary, precipitation or crystallization of the preferred enantiomer so formed as a salt with the chiral auxiliary, and separation of the precipitated or crystallized salt from its mother liquor, respectively.

Typically, interconversion of (2R)- and (2S)-enantiomers (or (3R)- and (3S)-enantiomers) of the substituted 2-trifluoromethyl-2H-chromene-3-carboxylic acid or derivative thereof is done at a temperature of from about −30° C. to about 200° C. Optionally the interconversion of the enantiomers of the substituted 2-trifluoromethyl-2H-chromene-3-carboxylic acid or derivative thereof is done at a temperature above room temperature, typically from about 25° C. to about 150° C., from about 25° C. to about 125° C., from about 25° C. to about 100° C., from about 30° C. to about 100° C., from about 35° C. to about 100° C., from about 40° C. to about 100° C., from about 50° C. to about 100° C., or from about 60° C. to about 100° C.

Preferred is light-promoted interconversion of (2R)- and (2S)-enantiomers (or (3R)- and (3S)-enantiomers) of the substituted 2-trifluoromethyl-2H-chromene-3-carboxylic acid or derivative thereof dissolved in a dilute solution, typically at concentrations of less than 100 grams of acid or derivative per liter of solution (“g/L”). Preferably, the solution comprises mobile phase useful in the method of the present invention or a component thereof.

Steady state recycling chromatography includes SSRC known by the trade name CYCLOJET® (Novasep Societe Par Actions Simpliflee, Pompey, France) and by the trademark “SteadyCycle™” (CYBA Technologies, LLC, Mystic, Conn., USA).

For purposes of characterizing a particular separation, a number of values known in the art may be determined. For example:

The capacity factor k′ for Peak n (“k′ₙ”)=[(retention volume of peak n) minus (dead time)] divided by (dead time). k′ for Peak n relates to how long Peak n is retained on a column; the longer Peak n is retained, the higher k′.

The separation factor (also known as the selectivity factor) α=[(capacity factor k′₁ for the more strongly retained component) divided by (capacity factor k′₂ for the less strongly retained component)]. α is always greater than 1.

The phrase “chiral stationary phase” includes a solid support and a chiral adduct such as a polysaccharide-, (tartaric acid)-, poly[(S)-N-acryloylphenylal amine ethyl ester-, 3,5-dinitrobenzoyl-phenylglycine-, L-proline bound to polyacrylamide-, vancomycin- or Λ-tris(1,10-phenanthroline)ruthenium sodium magnesium-derived chiral adduct, and the like, wherein the chiral adduct may or may not be covalently bound to the solid support.

The chiral polysaccharide stationary phases include silica gel supporting microcrystalline cellulose-triacetate, silica gel supporting a cellulose derivative wherein each glucose monomeric unit thereof is substituted with three 3,5-dimethylphenyl carbamate groups, silica gel supporting a cellulose derivative wherein each glucose monomeric unit thereof is substituted with three 4-methylbenzoyl groups, silica gel supporting an amylose derivative wherein each glucose monomeric unit thereof is substituted with three 3,5-dimethylphenyl carbamate groups, and silica gel supporting an amylose derivative wherein each glucose monomeric unit thereof is substituted with three (S)-alpha-phenethylcarbamate groups. Alternatively, the chiral polysaccharide stationary phase may comprise a synthetically made polysaccharide, a naturally occurring polysaccharide, or a modified version of a naturally occurring polysaccharide, or a polysaccharide selected from amylosic, cellulosic, chitin, chitosan (e.g., beta-1,4-chitosan), xylan (e.g., beta-1,4-xylan), curdan, mannan (e.g., beta-1,4-mannan), dextran, glucan (e.g., alpha-1,3-glucan and beta-1,3-glucan), and inulin class of polysaccharides. Alternatively, the chiral polysaccharide stationary phase may comprise a polysaccharide selected from cellulose tribenzoate, cellulose tricinnamate, amylose tricinnamate, amylose tris[(S)alpha-methyl benzyl carbamate], amylose 3,4-disubstituted phenyl carbamate, amylose (3-chloro-4-methylphenylcarbamate), amylose (4-chloro-3-methylphenylcarbamate), amylose (3-fluoro-4-methylphenylcarbamate), and amylose 4-substituted phenylcarbamate.

Alternatively, the chiral polysaccharide stationary phase comprising silica gel supporting a microcrystalline cellulose triacetate derivative is MCTA or CTA-I (Merck).

Alternatively, the chiral polysaccharide stationary phase comprising silica gel supporting a cellulose derivative wherein each glucose monomeric unit thereof is substituted with three benzoyl groups is CHIRALCEL® OB™ (Chiral Technologies, Inc., Exton, Pa.). Alternatively, the chiral polysaccharide stationary phase comprising silica gel supporting a cellulose derivative wherein each glucose monomeric unit thereof is substituted with three 4-chlorophenylaminocarbonyl groups is CHIRALCEL® OF™ (Chiral Technologies, Inc., Exton, Pa.). Alternatively, the chiral polysaccharide stationary phase comprising silica gel supporting a cellulose derivative wherein each glucose monomeric unit thereof is substituted with three 3,5-dimethylphenyl carbamate groups is CHIRALCEL® OD™ (Chiral Technologies, Inc., Exton, Pa.). Alternatively, the chiral polysaccharide stationary phase comprising silica gel supporting a cellulose derivative wherein each glucose monomeric unit thereof is substituted with three 4-methylbenzoyl groups is CHIRALCEL® OJ™ (Chiral Technologies, Inc., Exton, Pa.). Alternatively, the chiral polysaccharide stationary phase comprising silica gel supporting an amylose derivative wherein each glucose monomeric unit thereof is substituted with three 3,5-dimethylphenyl carbamate groups is CHIRALPAK® AD™ (Chiral Technologies, Inc., Exton, Pa.). Alternatively, the chiral polysaccharide stationary phase comprising silica gel supporting an amylose derivative wherein each glucose monomeric unit thereof is substituted with three (S)-alpha-phenethylcarbamate groups is CHIRALPAK® AS™ or CHIRALPAK® AS-V™ (Chiral Technologies, Inc., Exton, Pa.). Alternatively, the chiral polysaccharide stationary phase is from Daicel Chemical Industries Ltd., Japan. These stationary phases are described in U.S. Pat. Nos. 4,912,205 and 5,434,299.

The chiral tartaric acid stationary phases include silica gel supporting O,O′-bis (3,5-dimethylbenzoyl)-N,N′-diallyl-L-tartar diamide that is polymerized with a multifunctional hydrosilane to give a covalently bound chiral material or silica gel supporting O,O′-bis (4-tert-butylbenzoyl)-N,N′-diallyl-L-tartar diamide that is polymerized with a multifunctional hydrosilane to give a covalently bound chiral material.

Alternatively, the chiral tartaric acid stationary phase comprising silica gel supporting O,O′-bis (3,5-dimethylbenzoyl)-N,N′-diallyl-L-tartar diamide that is polymerized with a multifunctional hydrosilane to give a covalently bound chiral material is KROMASIL® DMB (Eka Nobel AB, Bohus, Sweden). Alternatively, the chiral tartaric acid stationary phase comprising silica gel supporting O,O′-bis (4-tert-butylbenzoyl)-N,N′-diallyl-L-tartar diamide that is polymerized with a multifunctional hydrosilane to give a covalently bound chiral material is KROMASIL® TBB (Eka Nobel AB, Bohus, Sweden).

The chiral poly[(S)-N-acryloylphenylal amine ethyl ester-derived polyacrylamide/silica composite stationary phases include CHIRASPHER® (Merck KGAA Limited Partnership, Darmstadt, Federal Republic of Germany) available from E. Merck.

The chiral 3,5-dinitrobenzoyl-phenylglycine-derived stationary phases are π-acidic and π-basic (Pirkle-type) phases that include DNBPG available from Regis Technologies, Inc., Morton Grove, Ill., United States of America.

The chiral L-proline bound to polyacrylamide-derived stationary phases include CHIROSOLVE® PRO (Ychem International, Niteen A. Vaidya, citizen of the United States, SOLE PROPRITORSHIP CALIFORNIA, 616 Stendhal Lane, Cupertino, CALIFORNIA, 95014) available from JPS Chemie Knoll AG ZA Ltd.

The chiral Λ-tris(1,10-phenanthroline)ruthenium sodium magnesium-derived stationary phases are metal complex based phases that include Ceramo-sphere available from Shiseido Company, Ltd., Japan.

The chiral stationary phase in a method of the present invention may comprise a solid support selected from silica gel, zirconium, magnesia, titanium oxide, glass, kaolin, alumina, a ceramic, and a silica other than silica gel. Alternatively, the chiral stationary phase may comprise a solid support selected from polystyrene, polyacrylamide, and polyacrylate.

The phrase “reverse phase, chiral stationary phase” means an immobile phase suitable for reverse phase enantioselective liquid chromatography, comprising a solid support and a chiral adduct, wherein the chiral adduct may or may not be covalently bound to the solid support.

The reverse phase, chiral stationary phases useful in a method of the present invention include a chiral α-cyclodextrin stationary phase, a chiral β-cyclodextrin stationary phase, a chiral γ-cyclodextrin stationary phase, a chiral macrocyclic glycopeptide stationary phase, a chiral D-amine stationary phase, a chiral α1-acid glycopeptide stationary phase, a chiral cellobiohydrolase stationary phase, and a chiral human serum albumin stationary phase. Preferred is a chiral cyclodextrin stationary phase or a chiral macrocyclic glycopeptide stationary phase.

Alternatively, the chiral α-cyclodextrin stationary phase is CYCLOBOND III (Advanced Separation Technologies, Inc., Whippany, N.J.). Alternatively, the chiral β-cyclodextrin stationary phase is CYCLOBOND I 2000 or CYCLOBOND I 2000 DM (Advanced Separation Technologies, Inc., Whippany, N.J.). Alternatively, the chiral γ-cyclodextrin stationary phase is CYCLOBOND II (Advanced Separation Technologies, Inc., Whippany, N.J.). Alternatively, the chiral macrocyclic glycopeptide (e.g., vancomycin) stationary phase is CHIROBIOTIC® V, CHIROBIOTIC® T, CHIROBIOTIC® TAG, or CHIROBIOTIC® R (all by Advanced Separation Technologies, Inc., Whippany, N.J.). Alternatively, the chiral D-amine stationary phase is ASTEC CLC-D (Advanced Separation Technologies, Inc., Whippany, N.J.). Alternatively, the chiral α1-acid glycopeptide stationary phase is CHIRAL-AGP® (ChromTech, Ltd., Cheshire, United Kingdom). Alternatively, the chiral cellobiohydrolase stationary phase is CHIRAL-CBH (Advanced Separation Technologies, Inc., Whippany, N.J.). Alternatively, the chiral human serum albumin stationary phase is CHIRAL-HSC (Advanced Separation Technologies, Inc., Whippany, N.J.). Alternatively, the chiral cyclodextrin stationary phase is CYCLOSE® (Chiralsep Corporation, La Frenaye, France) available from ChiralSep, Nucleodex available from MACHEREY-NAGEL Inc., Easton, Pa., or CHIRASEP® (E. Merck offene Handelsgesellschaft (o.H.G.), Darmstadt, Federal Republic of Germany) available from YMC, Inc in the United States of America and YMC Europe GmbH, Federal Republic of Germany.

The amount of chiral adduct on solid support in a method of the present invention will typically be from about 1% weight/weight (“wt/wt”) to about 99% wt/wt, typically from about 5% wt/wt to about 50% wt/wt, more typically from about 15% wt/wt to about 30% wt/wt. Preferred are stationary phases having a chiral adduct that is substantially homogeneous.

Stationary phase particle size in a method of the present invention is from about 1 micrometer (“μm”) to about 300 μm, typically from about 1 μm to about 100 μm, from about 5 μm to about 75 μm. Alternatively, the particle size generally is from about 1 μm to about 10 mm, typically from about 1 μm to about 300 μm, from about 2 μm to about 100 μm, from about 5 μm to about 75 μm, or from about 10 μm to about 30 μm.

The particles of the stationary phases useful in a method of the present invention may be poreless or porous. When the particles are porous, the average diameter of the pores typically ranges from about 10 angstroms (“A”) to about 10,000 Å, more typically from about 200 Å to about 2,000 Å.

The acid or derivative thereof in a method of the present invention may be a compound of Formula I″, I′, or I. Alternatively, the acid or derivative thereof may be a compound of Formula II.

Another aspect of this invention is any one of the above or below methods for separating enantiomers of a substituted 2-trifluoromethyl-2H-chromene-3-carboxylic acid or derivative thereof, wherein the mixture of the enantiomers comprises a compound of Formula I″, I′, or I wherein X is O.

Another aspect of this invention is any one of the above or below methods for separating enantiomers of a substituted 2-trifluoromethyl-2H-chromene-3-carboxylic acid or derivative thereof, wherein the mixture of the enantiomers comprises a compound of Formula II wherein X is O.

A method of the present invention may further comprise the step of isolating in a form that is substantially free of mobile phase, at least one of the separated enantiomers. Alternatively, the method may further comprise the step of isolating at least one of the separated enantiomers wherein the mobile phase containing at least one of the separated enantiomers is kept substantially free of ultraviolet or visible light during the isolation.

A method of the present invention includes eluting at least one of the separated enantiomers in at least 80%, 90%, 95% ee, 97% ee, 98% ee, or 99% ee. Alternatively, at least one of the separated enantiomers is isolated in at least 80%, 90%, 95% ee, 97% ee, 98% ee, or 99% ee after a subsequent enantioselective fractional crystallization of the separated enantiomer.

A method of the present invention includes recovering at least one of the separated enantiomers from the mobile phase in at least 90% ee and at least 50%, 60%, 70%, 80%, 90%, 95%, 97%, 98%, 99%, 125%, 150%, 175%, or 190% yield (number of moles of a separated enantiomer divided by number of moles of the enantiomer introduced for the first time to a stationary phase, times 100). Recovery yields of greater than 100% may be obtained if the antipode of the enantiomer is interconverted to give a new mixture of enantiomers, and the new mixture is separated according to a method of the present invention. The interconversion step and recycle of the new mixture can be repeated from 1 to more than 200 times to maximize recovery yield of the separated enantiomer. Alternatively, at least one of the separated enantiomers is recovered from the mobile phase in at least 98% ee and at least 50%, 60%, 70%, 80%, 90%, 95%, 97%, 98%, 99%, 125%, 150%, 175%, or 190% yield following a subsequent enantioselective fractional crystallization of the separated enantiomer.

A method of the present invention may further comprise a step of subjecting one of the enantiomers or a non-racemic mixture of the enantiomers to interconversion by UV or visible light irradiation. The method may further comprise a step of resubjecting the resulting mixture of enantiomers to an enantioselective multicolumn chromatography. Preferred is wherein the resulting interconverted mixture is resubjected to the multicolumn chromatography via a recycle stream or recycle/feed stream.

The introduction of the mixture (feed stream, recycle stream, and the like) in a method of the present invention is continuous, semi-continuous, or discontinuous.

A method of the present invention may further comprise a preliminary step of subjecting the mixture of the enantiomers to enantioselective fractional crystallization with or without a chiral auxiliary.

A method of the present invention may further comprise a subsequent step of subjecting the mixture of the enantiomers to enantioselective fractional crystallization with or without a chiral auxiliary.

A method of the present invention may further comprise a preliminary step of subjecting the mixture of the enantiomers to enantioselective fractional crystallization with or without a chiral auxiliary and an independent subsequent step of subjecting the mixture of the enantiomers to enantioselective fractional crystallization with or without a chiral auxiliary.

Chiral auxiliaries include, but are not limited to, (S)(−)-α-methylbenzylamine, (−)cinchonidine, and (S)(−)-2-amino-3-phenyl-1-propanol.

In a method of the present invention, the mixture of the enantiomers may be introduced to the stationary phase as a solution of the mixture dissolved in the mobile phase or as a solution of the mixture dissolved in a solvent or solvent mixture that is compatible with the method of the present invention, wherein the solvent or solvent mixture is not the mobile phase. The solution includes a feed solution and a recycle solution.

In a method of the present invention wherein the enantiomers are of a substituted 2-trifluoromethyl-2H-chromene-3-carboxylic acid or acid derivative thereof, the mobile phase may comprise a polar solvent, wherein the polar solvent contains from 1 to 8 carbon atoms and 1 oxygen atom and is selected from straight or branched acyclic C₁-C₈ alcohols such as methanol, ethanol, propanol, iso-propyl alcohol, butanol, and the like, cyclic C₃-C₈ alcohols such as cyclopropanol, cyclobutanol, and the like, C₄-C₈ ethers such as ethyl ether, tert-butyl methyl ether, tetrahydrofuran, tetrahydropyran, and the like, straight or branched C₃-C₈ alkanones such as acetone, butanone, 2-pentanone, 3-pentanone, 3,3-dimethyl-2-pentanone, and the like, and C₃-C₈ cycloalkanones such as cyclopropanone, cyclobutanone, cyclopentanone, cyclohexanone, 3-methylcyclopentanone, and the like.

Alternatively wherein the enantiomers are of a substituted 2-trifluoromethyl-2H-chromene-3-carboxylic acid or acid derivative thereof, the mobile phase may comprise a polar solvent, wherein the polar solvent contains from 1 to 8 carbon atoms and 2 oxygen atoms and is selected from supercritical fluid such as carbon dioxide, C₃-C₈ esters such as methyl acetate, ethyl acetate, propyl propionate, methyl butyrate, and the like, C₃-C₈ lactones such as beta-butyrolactone, gamma-butyrolactone, gamma-valerolactone, delta-valerolactone, and the like, and C₃-C₈ bis ethers such as 2-methoxy-ethyl ether, and the like.

Alternatively wherein the enantiomers are of a substituted 2-trifluoromethyl-2H-chromene-3-carboxylic acid or acid derivative thereof, the mobile phase may comprise a polar solvent, wherein the polar solvent contains from 1 to 8 carbon atoms and 1 nitrogen atom and is selected from C₂-C₈ nitriles such as acetonitrile, propionitrile, butyronitrile, and the like.

Alternatively wherein the enantiomers are of a substituted 2-trifluoromethyl-2H-chromene-3-carboxylic acid or acid derivative thereof, the mobile phase may comprise a polar solvent, wherein the polar solvent contains from 1 to 8 carbon atoms, 1 oxygen atom, and 1 nitrogen atom and is selected from C₂-C₈ carboxylic amides such as C₂-C₈ amides such as acetamide, N-methyl-acetamide, N,N-dimethylformamide, butyramide, and the like and C₄-C₈ lactams such as beta-lactam, 2-pyrrolidinone, 1-methyl-2-pyrrolidinone, delta-valerolactam, and the like.

Alternatively wherein the enantiomers are of a substituted 2-trifluoromethyl-2H-chromene-3-carboxylic acid or acid derivative thereof, the mobile phase may comprise a polar solvent, wherein the polar solvent contains from 1 to 8 carbon atoms and 2 or 3 chlorine atoms and is selected from dichloro-(C₁-C₈ hydrocarbons) such as dichloromethane, and trichloro-(C₁-C₈ hydrocarbons) such as 1,1,1-trichloroethane, and the like.

Alternatively wherein the enantiomers are of a substituted 2-trifluoromethyl-2H-chromene-3-carboxylic acid or acid derivative thereof, the mobile phase may comprise an acidic solvent, wherein the acidic solvent is selected from an acyclic unsubstituted C₁-C₈ carboxylic acid that is straight or branched such as formic acid, acetic acid, propionic acid, and the like and a C₃-C₈ cyclic carboxylic acids such as cyclopropyl-carboxylic acid, 3-methyl-cyclobutylcarboxylic acid, and the like.

Alternatively wherein the enantiomers are of a substituted 2-trifluoromethyl-2H-chromene-3-carboxylic acid or acid derivative thereof, the mobile phase may comprise an acidic solvent, wherein the acidic solvent is selected from an acyclic C₁-C₈ carboxylic acid that is straight or branched and substituted with from 1 to 3 fluoro such as trifluoroacetic acid, and the like, an acyclic C₁-C₈ carboxylic acid that is straight or branched and substituted with from 1 to 3 chloro such as chloroacetic acid, trichloroacetic acid, and the like, and an acyclic C₁-C₈ carboxylic acid that is straight or branched and substituted with 1 bromo such as bromoacetic acid, and the like.

Alternatively wherein the enantiomers are of a substituted 2-trifluoromethyl-2H-chromene-3-carboxylic acid or acid derivative thereof, the mobile phase may comprise an acidic solvent, wherein the acidic solvent is selected from an acyclic unsubstituted C₁-C₈ sulfonic acid that is straight or branched, such as methanesulfonic acid, 2,2,2-trimethylmethanesulfonic acid, and the like.

Alternatively wherein the enantiomers are of a substituted 2-trifluoromethyl-2H-chromene-3-carboxylic acid or acid derivative thereof, the mobile phase may comprise an acidic solvent, wherein the acidic solvent is selected from an acyclic C₁-C₈ sulfonic acid that is straight or branched and substituted with from 1 to 3 fluoro such as fluoromethanesulfonic acid, difluoromethanesulfonic acid, trifluoromethanesulfonic acid, 3,3,3-trifluoropropanesulfonic acid, and the like.

Alternatively wherein the enantiomers are of a substituted 2-trifluoromethyl-2H-chromene-3-carboxylic acid or acid derivative thereof, the mobile phase may comprise a nonpolar solvent that is a straight chain or branched C₅-C₁₀ acyclic hydrocarbon comprises n-pentane, iso-pentane, n-hexane, n-heptane, 2,2,5-trimethylhexane, and the like.

Alternatively wherein the enantiomers are of a substituted 2-trifluoromethyl-2H-chromene-3-carboxylic acid or acid derivative thereof, the mobile phase may comprise a nonpolar solvent that is a C₅-C₁₀ cyclic hydrocarbon comprises cyclopentane, cyclohexane, methylcyclopentane, cycloheptane, and the like.

The present invention includes methods wherein the composition of the mobile phase does not change during the time course of the elution. Alternatively, the elution is a gradient elution with a solution comprising a polar solvent, an acidic solvent, and a nonpolar solvent such that the composition of the mobile phase changes by gradually increasing the proportion of the polar solvent relative to the nonpolar solvent and acidic solvent. Alternatively, the elution is a gradient elution with a solution comprising a polar solvent and an aqueous solution such that the elution is a gradient elution with a mobile phase that gradually increases in the proportion of the polar solvent relative to the aqueous solution.

In a method of the present invention wherein the enantiomers are of a substituted 2-trifluoromethyl-2H-chromene-3-carboxylic ester or ester derivative thereof, the mobile phase may be selected from: a single polar solvent and a solution comprising a polar solvent and a nonpolar solvent wherein the polar solvent is less than or equal to 50% volume/volume of the miscible mixture and the nonpolar solvent is greater than 50% volume/volume of the solution. The polar solvent and nonpolar solvent are as defined above. Alternatively, the mobile phase may be a supercritical fluid.

In a method of the present invention wherein the enantiomers are of a pharmaceutically acceptable salt of a substituted 2-trifluoromethyl-2H-chromene-3-carboxylic acid or derivative thereof, the mobile phase may comprise the buffered neutral aqueous solution comprises water and a salt such as a sodium or potassium perchlorate, biphosphate, phosphate, bisulfate, sulfate, and the like. The buffered acidic aqueous solution comprises water, a salt such as a sodium or potassium perchlorate, biphosphate, phosphate, bisulfate, sulfate, and the like and an acid selected from formic acid, acetic acid, trifluoroacetic acid, phosphoric acid, sulfuric acid, and the like. The buffered basic aqueous solution comprises water, a salt such as a sodium or potassium perchlorate, biphosphate, phosphate, bisulfate, sulfate, and the like and a base selected from sodium acetate, potassium acetate, sodium hydroxide, potassium hydroxide, and the like. The polar solvent is selected from a C₃-C₆ alkanone such as acetone, a C₂-C₆ nitrile such as acetonitrile, and a C₁-C₆ alcohol such as methanol, ethanol, 1-propanol, 2-propanol, 1-butanol, 2-butanol, and the like. The polar solvent may comprise from about 1% to about 99%, from about 5% to about 95%, from about 10% to about 90%, from about 20% to about 80%, or from about 30% to about 70% volume/volume of the mobile phase.

The method of the present invention includes enantioselective chromatographies conducted in Polar Organic mode utilizing a mobile phase comprising a polar solvent such as ethanol, methanol, or acetonitrile and optionally an acidic solvent such as trifluoroacetic acid or acetic acid.

The method of the present invention includes enantioselective steady state recycling chromatography using two columns and enantioselective steady state recycling chromatography using a single column.

In typical enantioselective HPLC or enantioselective SSRC, columns containing a chiral stationary phase are typically dimensioned to about a 4.6 millimeter (“mm”) inner diameter (“i.d.”) and a 250 mm length. In an enantioselective batch preparative HPLC, columns containing a chiral stationary phase are typically dimensioned to about 5 cm i.d. to about 100 cm length, more typically from about 7 cm i.d. to about 75 cm length, from about 9 cm i.d. to about 60 cm length.

In an enantioselective HPLC or enantioselective SSRC of the present invention, preferred is a method that affords mass balance recovery of at least 80% of at least one purified enantiomer (e.g., at least 40% mass balance starting from a racemic mixture) wherein the recovered material contains no more than 2.5% of the opposite enantiomer. In a typical laboratory embodiment, the method of the present invention is one that affords mass balance recovery of at least 90% of at least one purified enantiomer that is essentially free of its opposite enantiomer.

In a typical enantioselective HPLC separation, a mixture of enantiomers of a substituted 2-trifluoromethyl-2H-chromene-3-carboxylic acid or derivative thereof, is dissolved in a compatible solvent or solvents mixture at a concentration of about 1 g/L before introduction to the chromatography pathway. Typically, this solution is introduced to the chromatography pathway by injection via an injection port, wherein the stationary phase and mobile phase in the column have typically been equilibrated prior to injection. Equilibration typically is carried out by passing mobile phase through the column containing stationary phase for from about 30 minutes to about 3 hours prior to injection. The enantiomers are then eluted with mobile phase at a rate that produces an eluate flow rate of about 1 milliliter per minute (“mL/min.”). Separations are typically conducted at ambient temperature (i.e., from about 20° C. to about 40° C.).

The phrase “multicolumn chromatography” means a chromatography method that utilizes more than one column connected in series. Typically MCC is most productive (as measured by weight of mixture of enantiomers to be separated per unit weight of stationary phase) when operated in a continuous mode. MCC performed in a semi-continuous or discontinuous mode is also embraced by the method of the present invention.

Typically, from 3 to 200 columns may be used in an enantioselective MCC. The columns are connected in series in an enantioselective MCC pathway such that the outlet of each column is connected to the inlet of the next column in the series and the outlet of the last column in the series is connected to the inlet of the first column. Other connections may be made into this pathway to allow for feed, raffinate, extract, and recycle streams. Optimization of parameters for an enantioselective MCC may be carried out experimentally or by using a simulation tool such as the methodology based on modeling and simulation of non-linear chromatography described by Charton F. and Nicoud, R. M., J. Chrom., 1995; 702:97-112.

Other aspects of multicolumn chromatography include asynchronous multicolumn chromatography, wherein the switching of inlet and outlet lines is not done simultaneously, SMB chromatography, wherein the switching of inlet and outlet lines is done substantially simultaneously, and preparative scale, supercritical fluid chromatography (“PS-SFC”), which can be carried out using a substantially simultaneous or asynchronous switching mode. The method of the present invention includes enantioselective asynchronous MCC, SMB chromatography, and PS-SFC.

Asynchronous MCC includes, but is not limited to, VARICOL® (Novasep Societe Par Actions Simplifee, Pompey, France) MCC.

Enantioselective asynchronous multicolumn chromatography may be performed using a series of from 3 to 200 chromatography columns, typically 3, 4, 5, 6, 7, 8, 9, 10, 11, 12, 13, 14, 15, 16, 17, 18, 19, 20, 22, 24, 26, 28, 30, 32, from 3 to 100, 34 to 64, 3 to 52, 3 to 44, 3 to 40, 3 to 36, 3 to 32, 3 to 28, 3 to 24, 3 to 20, 4 to 30, 4 to 24, 4 to 20, 4 to 16, 5 to 32, 5 to 24, 5 to 20, 5 to 16, 5 to 32, 6 to 32, 6 to 24, 6 to 20, 7 to 18, or 7 to 16.

In asynchronous MCC, the column distribution between zones thus varies over time. Asynchronous MCC is typically a periodic process in that it returns to the same status at the end of a period. In one aspect, the period is composed of two equal time intervals (i.e., switch times are the same). However, many other situations could obviously be envisioned, including situations wherein switch times are different. The number of column configurations in asynchronous MMC is infinite because it is not necessary to have an integer number of columns per zone.

In an asynchronous MCC example having a period consisting of two equal time intervals, a mobile phase make-up inlet, a feed inlet, an extract outlet, and a raffinate outlet, at time=0 an initial column configuration is characterized by no column in a first zone, two columns in a second zone, one column in a third zone, and one column in a fourth zone. At this point, there is no column separating the mobile phase make-up inlet from the extract outlet. This column distribution stays the same for the first time interval.

At the end of the first time interval, the extract and raffinate lines are simultaneously shifted, whereas the mobile phase make-up and feed lines are not shifted. This means that one column is now assigned to the first zone (between the eluent extract lines), one column is in the second zone, two columns are in the third zone, and there is no column in the fourth zone. At this point, there is no column separating the raffinate outlet and mobile phase make-up inlet. This column distribution stays constant until the end of the second time interval, at which point the eluent and feed lines are shifted by one column and the original status (same as at t=0) is restored. The column distribution pattern is repeated during at the following periods as the chromatography proceeds.

The column distribution across zones over a period is characterized by the average number of columns per zone. In the above example, the first and fourth zones contain an average of 0.5 columns each over the period. Similarly, the second and third zones contain an average of 1.5 columns each over the period.

The temporary superposition of some lines (extract outlet and mobile phase make-up inlet, and raffinate outlet and mobile phase make-up inlet in the above example) leads to obvious consequences for the technical design of an asynchronous MCC pathway. Between each column, the two outlets lines (extract and raffinate) must be connected before the tow inlet lines (mobile phase make-up and feed) following the direction of the recycling stream.

Using this design, the asynchronous MCC feed stream does not pollute the extract or raffinate streams when the number of columns is temporarily zero in the second and third zone, respectively. Similarly, the mobile phase make-up stream does not unacceptably dilute the extract or raffinate stream when the number of columns is zero in the first and fourth zones.

In simulated moving bed chromatography, process features include countercurrent flow of mobile phase and stationary phase without the actual movement of the stationary phase (i.e., a simulated moving bed). The counter-current movement of the solid phase with respect to the liquid phase is simulated by periodically and substantially simultaneously switching all inlet (feed and recycle streams) and outlet (extract and raffinate streams) lines in the direction of the liquid flow. In one aspect of SMB chromatography, the adsorption and desorption operations are continuously occurring, which allows both continuous production of extract and raffinate streams and the continual use of feed and desorbant streams, and optionally a recycle stream. There are various aspects of enantioselective SMB chromatography, including enantioselective high performance SMB (“HP-SMB”). A typical enantioselective SMB chromatography pathway has four zones with at least one column per zone, more typically at least two columns per zone.

In an illustrative example of SMB chromatography, initially if mobile phase is introduced in column 4, eluent in column 1, raffinate is removed from column 8, and extract is removed from column 2, then at the expiration of a next switch time, mobile phase will be introduced in column 5, eluent in column 2, raffinate will be removed from column 1, and extract will be removed from column 3, and at the expiration of another next switch time, the column introductions and removal positions will advance again by one column, and so on.

The method of the present invention includes enantioselective simulated moving bed chromatography. Enantioselective simulated moving bed chromatography may be performed using a series of from 4 to 200 chromatography columns, typically 4, 6, 8, 10, 12, 14, 16, 18, 20, 22, 24, 26, 28, 30, 32, from 4 to 100, 4 to 64, 4 to 52, 4 to 44, 4 to 40, 4 to 36, 4 to 32, 4 to 28, 4 to 24, 4 to 20, 8 to 30, 8 to 24, 8 to 20, 8 to 16, 12 to 32, 12 to 24, 12 to 20, 12 to 16, 16 to 32, 16 to 32, 16 to 24, 12 to 20, 12 to 18, or 12 to 16.

In a typical enantioselective SMB chromatography using 8 columns in cyclic 1-2-3-4-5-6-7-8-1-etc. pathway, for example, mobile phase may be introduced to the pathway before column 1, extract may be removed from the pathway from column 2 (i.e., before column 3), the feed containing a mixture of the enantiomers in a compatible solvent, typically mobile phase for SMB chromatography, may be introduced to the pathway before column 5, and raffinate may be removed from the pathway from column 6 (i.e., before column 7). Optionally, the extract or raffinate may be cycled through an interconversion unit if desired and reintroduced to the chromatography pathway by mixing into the racemic feed stream. If the interconverted mixture of enantiomers is recycled and introduced into the racemic feed stream, the fresh racemic feed flow rate may be reduced. Alternatively, the interconverted mixture may dissolve additional fresh mixture of enantiomers to increase the concentration of enantiomers to a more optimum concentration for a productive separation, which is then fed into the chromatography pathway as a recycle/feed stream.

A typical column for enantioselective MCC is dimensioned larger, such as about 2.6 centimeters (“cm”) inner diameter and about 10.7 cm length or about 4.8 cm inner diameter and 11 cm length. In manufacturing setting, a column for enantioselective MCC may be dimensioned even larger such as about a 1-meter i.d. and about a 12 cm length.

Columns in an enantioselective MCC method of the present invention may be connected with polymer capillaries such as polyetheretherketone (“PEEK”) or Tefzel capillaries, stainless steel capillaries, and the like. Preferred are stainless steel capillaries.

In enantioselective MCC wherein the less strongly retained enantiomer is the preferred one, typically there are at least two options for interconversion of a less preferred enantiomer: Option 1: the less strongly retained enantiomer is preferred and mostly recovered from the raffinate stream and the extract stream contains mostly the more strongly retained enantiomer, which is interconverted and recycled. Option 2: the less strongly retained enantiomer is preferred and partially recovered in the raffinate and the extract contains significant amounts of both enantiomers, which are interconverted and recycled. In any event, either the raffinate stream or the extract stream may be recycled through an interconversion unit to interconvert the less preferred enantiomer.

In typical enantioselective MCC method, a mixture of enantiomers of a substituted 2-trifluoromethyl-2H-chromene-3-carboxylic acid or derivative thereof, is introduced to the chromatography pathway dissolved in a compatible solvent or solvent mixture at a concentration of from about 5 g/L to about 100 g/L, typically from about 10 g/L to about 80 g/L, and the like.

Feed solutions of the mixture of enantiomers are typically introduced into an enantioselective MCC pathway through a feed port at a typical flow rate of from about 5 mL/min. to about 100 mL/min., more typically from about 10 mL/min. to about 75 mL/min., and the like. Mobile phase is typically introduced through a port at a typical flow rate of from about 15 mL/min. to about 300 mL/min., more typically from about 20 mL/min. to about 150 mL/min., and the like. Other ports allow removal of raffinate and extract streams. Extract flow rates typically are from about 20 mL/min. to about 300 mL/min., more typically from about 20 mL/min. to about 150 mL/min., and the like whereas raffinate flow rates are typically from about 10 mL/min. to about 100 mL/min., typically from about 12 mL/min. to about 80 mL/min., and the like. Still another port allows recycling of eluate through the chromatography pathway, if desired. Recycling flow rates typically are from about 20 mL/min. to about 500 mL/min., typically from about 30 mL/min. to about 450 ml/min., from about 50 mL/min. to about 350 mL/min. The above-recited flow rates are for illustration purposes only. Actual flow rates will depend upon a number of factors, including separation, the dimensions of the columns being used, such as cross-sectional areas, the particular stationary phase being employed, particle size of the stationary phase, viscosity of the mobile phase, and the like. Accordingly, flow rates may be above or below the above-recited values by up to a factor of 2 or 0.5, respectively.

Equilibration for enantioselective MCC typically is carried out by passing mobile phase through the columns containing stationary phase for from about 30 minutes to about 3 hours prior to injection.

Separations using MCC are typically conducted at temperatures from about 5° C. to about 50° C., typically from about 15° C. to about 40° C.

Switch times in enantioselective MCC typically are from about 15 seconds to about 10 minutes, more typically from about 30 seconds to about 5 minutes, from about 40 seconds to about 3 minutes, from 45 seconds to about 2 minutes, and the like. Switch time is the time between switching flow between the column inlets and outlets. Switching of flows simulates a moving chromatography bed.

A preferred aspect of the present invention is enantioselective MCC that affords a total mass balance recovery of at least 90% of at least one purified enantiomer (e.g., at least 45% mass balance starting from a racemic mixture) wherein the recovered material is enriched to an extent that the purified enantiomer is present in at least about 95% enantiomeric excess, 97% e.e., 98% e.e., or 99% e.e. Alternatively, an enantioselective MCC is one that affords a total mass balance recovery of at least 95% of at least one purified enantiomer (e.g., at least 47.5% mass balance starting from a racemic mixture) wherein the recovered material is enriched to an extent that the purified enantiomer is present in at least about 95% e.e., 97% e.e., 98% e.e., or 99% e.e. and recovered material in 95% e.e. or 97% e.e. may optionally be further purified to at least about 98% enantiomeric excess by from 1 to 3 subsequent enantioselective fractional crystallizations with or without chiral auxiliary. Illustrative examples of the fractional co-crystallizations with chiral auxiliary are found in U.S. Pat. Nos. 6,077,850 and 6,034,256 as referenced above.

Liquid chromatography set-ups are commercially available from, for example, Advanced Separations Technologies, Inc., The Novasep Group (e.g., Licosep Lab unit), Agilent Technologies Inc. (formerly part of Hewlett-Packard), Palo Alto, Calif., Shimadzu, Columbia, Md., and Jasco, Easton, Maryland (e.g., a Jasco 880-PU liquid chromatography instrument with a RHEODYNE 7125 injector from Rheodyne Inc. and a ERC-3611 degasser from Erma CR, Inc.

A satisfactory separation of the enantiomers according to a method of the present invention may or may not mean a baseline separation. Separations that contain at least 10% of fractions that contain at least a 9:1 ratio of the enantiomers are satisfactory for practicing the method of the present invention.

The method of the present invention may be carried out in a continuous, semi-continuous, or discontinuous mode of operation. Discontinuous chromatographies include those modes of operation wherein a feed solution containing the mixture of the enantiomers or a recycle solution containing a partially separated mixture of the enantiomers is periodically, but not continuously, introduced into the chromatography pathway. True continuous chromatographies include those modes of operation wherein the feed solution or the recycle solution is essentially continuously introduced into the chromatography pathway from the start of the separation process until the end of the separation process. Semi-continuous chromatographies include those modes of operation that periodically proceed in a continuous mode and periodically proceed in a discontinuous mode.

The values provided above in reference to, for example, particle sizes, pore sizes, column dimensions, flow rates, and the like are given for illustrative purposes only and are not to be construed as limiting the method of the present invention in any respect. For purposes of practicing the method of the present invention, it is not critical that a particular pore size, particle size, flow rate, concentration of mixture of enantiomers, column dimension, numbers of columns in series, etc. be used so long as a satisfactory separation of the enantiomers is obtained.

A suitable chiral auxiliary for separating a mixture of enantiomers of a substituted 2-trifluoromethyl-2H-chromene-3-carboxylic acid or acid derivative thereof can be identified by screening. An illustrative screening procedure is charging 20 mL vials with 50 mg of the acid or acid derivative thereof, 1.0 or 0.5 mole equivalents of a basic chiral auxiliary, and 2 mL of a suitable solvent such as ethanol, methyl tert-butyl ether, heptane, or Isopar C, and cap and shake the vials on a shaker for 4 hours at room temperature. More solvent is added if there is solid remaining after the shaking period. If necessary, the volume of the mixture is then reduced by evaporation (e.g., under house vacuum or by a stream of nitrogen gas) until solids form. If no solids form after reducing the volume of the mixture by about 50%, the vials are cooled in a freezer. All mixtures with solids are filtered, and both solids and mother liquors are characterized by enantioselective HPLC according to a method of the present invention. Enriched preferred enantiomer may be found either in the solids or mother liquor, depending upon the particular separation method being conducted. For example, samples for characterization are diluted with 10% EtOH/heptane to a final concentration of about 0.5 mg/mL and analyzed with a CHIRALPAK® AD column (Daicel Chemical Industries, Ltd.) with detection at 254 nm. Mobile phase is, for example, heptane-ethanol-trifluoroacetic acid (95:5:0.1, volume proportions) pushed at a flow rate of 1.0 mL/minute and the injection volume is 10 microliters.

With the present invention in mind, one of ordinary skill in the art can determine suitable parameters and conditions for separating a particular enantiomeric mixture of the chromene derivatives without undue experimentation.

The method of the present invention includes analytical HPLC, preparative scale chromatography, and manufacturing scale chromatography.

The method of the present invention works whether the mixture of enantiomers of the substituted 2-trifluoromethyl-2H-chromene-3-carboxylic acids, and derivatives thereof, are free of impurities or not, free of water or other solvates or not, are crystalline or amorphous, are liquid or solid, and the like.

### EXAMPLES

Representative examples of the method of the present invention are described below. Chiral purities (e.g., enantiomeric purity and enantiomeric excess) of the enantiomers of the substituted 2-trifluoromethyl-2H-chromene-3-carboxylic acids and derivatives thereof described below in the examples were determined using enantioselective HPLC according to a method of the present invention. Absolute stereochemistries where reported below were determined by comparison of enantioselective HPLC retention times to those for the exact reference standards.

For Examples 1a to 19b, the retention times in minutes (“tR (min.)”) for the first eluting peak (“Peak 1”) and the second eluting peak (“Peak 2”), capacity factors for peak 1 (“k′₁”) and peak 2 (“k′₂”), and selectivity factors for the more strongly retained Peak 2 versus the less strongly retained Peak 1 (“α”) are shown below in Table 1.

### Examples 1a to 1i

Using a column with 0.46 cm inner diameter and 25 cm length filled with CHIRALPAK® AD stationary phase, racemic mixtures of (R)- and (S)-6-chloro-8-methyl-2-trifluoromethyl-2H-chromene-3-carboxylic acid were separated in separate experiments by eluting at room temperature with mobile phase (volume proportions) as follows:
1a: Heptane-2-propanol-trifluoroacetic acid (95:5:0.1),
1b: Methanol-trifluoroacetic acid (100:0.1),
1c: Heptane-ethanol-trifluoroacetic acid (95:5:0.1),
1d: Heptane-2-propanol-trifluoroacetic acid (95:5:0.1),
1e: Heptane-2-propanol-acetic acid (95:5:0.5),
1f: Heptane-2-propanol-acetic acid (95:5:0.25),
1g: Heptane-ethanol-trifluoroacetic acid (95:5:0.1),
1h: Heptane-ethanol-acetic acid (95:5:0.5), and
1i: Heptane-ethanol-acetic acid (95:5:0.25), respectively, at 1 mL/min. and detected with a photodiode array detector at 254 nm wavelength.

### Examples 2a to 2i

Using a column with 0.46 cm inner diameter and 25 cm length filled with CHIRALCEL® OJ stationary phase, racemic mixtures of (R)- and (S)-6-chloro-8-methyl-2-trifluoromethyl-2H-chromene-3-carboxylic acid were separated in separate experiments by eluting at room temperature with mobile phase (volume proportions) as follows:
2a: Heptane-ethanol-trifluoroacetic acid (95:5:0.1),
2b: Methanol-trifluoroacetic acid (100:0.1),
2c: Heptane-2-propanol-trifluoroacetic acid (95:5:0.1),
2d: Heptane-ethanol-trifluoroacetic acid (95:5:0.1),
2e: Heptane-ethanol-acetic acid (95:5:0.5),
2f: Heptane-ethanol-acetic acid (95:5:0.25),
2g: Heptane-2-propanol-trifluoroacetic acid (95:5:0.1),
2h: Heptane-2-propanol-acetic acid (95:5:0.5), and
2i: Heptane-2-propanol-acetic acid (95:5:0.25), respectively, at 1 mL/min. and detected with a photodiode array detector at 254 nm wavelength.

### Examples 3a to 3c

Using a column with 0.46 cm inner diameter and 25 cm length filled with CHIRALPAK® AD stationary phase, racemic mixtures of (R)- and (S)-8-chloro-6-methoxy-2-trifluoromethyl-2H-chromene-3-carboxylic acid were separated in separate experiments by eluting at room temperature with mobile phase (volume proportions) as follows:
3a: Heptane-2-propanol-trifluoroacetic acid (95:5:0.1),
3b: Methanol-trifluoroacetic acid (100:0.1), and
3c: Heptane-ethanol-trifluoroacetic acid (95:5:0.1), respectively, at 1 mL/min. and detected with a photodiode array detector at 254 nm wavelength.

### Example 4

Using a column with 0.46 cm inner diameter and 25 cm length filled with CHIRALPAK® AD stationary phase, a racemic mixture of (R)- and (S)-6-chloro-7-(1,1-dimethyl-2-hydroxyethyl)-2-trifluoromethyl-2H-chromene-3-carboxylic acid was separated by eluting at room temperature with mobile phase Heptane-2-propanol-trifluoroacetic acid (80:20:0.1) at 1 mL/min. and detected with a photodiode array detector at 210 nm wavelength.

### Examples 5a and 5b

Using a column with 0.46 cm inner diameter and 25 cm length filled with CHIRALCEL® OD stationary phase, racemic mixtures of (R)- and (S)-6-chloro-7-(1,1-dimethyl-2-hydroxyethyl)-2-trifluoromethyl-2H-chromene-3-carboxylic acid were separated in separate experiments by eluting at room temperature with mobile phase (volume proportions) as follows:
5a: Heptane-2-propanol-trifluoroacetic acid (90:10:0.1), and
5b: Heptane-ethanol-trifluoroacetic acid (90:10:0.1), respectively, at 1 mL/min. and detected with a photodiode array detector at 210 nm wavelength.

### Examples 6a to 6p

Using a column with 0.46 cm inner diameter and 25 cm length filled with CHIRALPAK® AD stationary phase, racemic mixtures of (R)- and (S)-6-chloro-7-tert-butyl-2-trifluoromethyl-2H-chromene-3-carboxylic acid were separated in separate experiments by eluting at room temperature with mobile phase (volume proportions) as follows:
6a: Heptane-2-propanol-trifluoroacetic acid (95:5:0.1),
6b: Heptane-2-propanol-acetic acid (95:5:0.5),
6c: Heptane-2-propanol-acetic acid (95:5:0.25),
6d: Heptane-2-propanol-trifluoroacetic acid (95:5:0.1),
6e: Heptane-ethanol-trifluoroacetic acid (95:5:0.1),
6f: Methanol-trifluoroacetic acid (100:0.1),
6g: Heptane-ethanol-trifluoroacetic acid (95:5:0.1),
6h: Heptane-ethanol-acetic acid (95:5:0.5),
6i: Heptane-ethanol-acetic acid (95:5:0.25),
6j: Methanol-trifluoroacetic acid (100:0.1),
6k: Methanol-acetic acid (100:0.25),
6l: Methanol-trifluoroacetic acid (100:0.1, polar organic mode),
6m: Methanol-acetic acid (100:0.5, polar organic mode),
6n: Methanol-acetic acid (100:0.25, polar organic mode),
6o: acetonitrile (100), and
6p: ethanol (100), respectively, at 1 mL/min. and detected with a photodiode array detector at 254 nm wavelength.

### Examples 7a and 7b

Using a column with 0.46 cm inner diameter and 25 cm length filled with CHIRALPAK® AS stationary phase, racemic mixtures of (R)- and (S)-6-chloro-7-tert-butyl-2-trifluoromethyl-2H-chromene-3-carboxylic acid were separated in separate experiments by eluting at room temperature with mobile phase (volume proportions) as follows:
7a: Methanol (100), and
7b: Ethanol (100), respectively, at 1 mL/min. and detected with a photodiode array detector at 254 nm wavelength.

### Examples 8a and 8b

Using a column with 0.46 cm inner diameter and 25 cm length filled with CHIRALCEL® OJ stationary phase, racemic mixtures of (R)- and (S)-6,8-dimethyl-2-trifluoromethyl-2H-chromene-3-carboxylic acid were separated in separate experiments by eluting at room temperature with mobile phase (volume proportions) as follows:
7a: Heptane-ethanol-trifluoroacetic acid (95:5:0.1), and
7b: Heptane-2-propanol-trifluoroacetic acid (95:5:0.1), respectively, at 1 mL/min. and detected with a photodiode array detector at 254 nm wavelength.

### Example 9

Using a column with 0.46 cm inner diameter and 25 cm length filled with CHIRALPAK® AD stationary phase, a racemic mixture of (R)- and (S)-6-chloro-7-benzyl-2-trifluoromethyl-2H-chromene-3-carboxylic acid was separated by eluting at room temperature with mobile phase Heptane-2-propanol-trifluoroacetic acid at 1 mL/min. and detected with a photodiode array detector at 280 nm wavelength.

### Example 10

Using a column with 0.46 cm inner diameter and 25 cm length filled with CHIRALPAK® AS stationary phase, a racemic mixture of (R)- and (S)-6-chloro-5,7-dimethyl-2-trifluoromethyl-2H-chromene-3-carboxylic acid was separated by eluting at room temperature with mobile phase Heptane-ethanol-trifluoroacetic acid (95:5:0.1) at 1 mL/min. and detected with a photodiode array detector at 254 nm wavelength.

### Examples 11a to 11d

Using a column with 0.46 cm inner diameter and 25 cm length filled with CHIRALPAK® AD stationary phase, racemic mixtures of (R)- and (S)-6-chloro-5,7-dimethyl-2-trifluoromethyl-2H-chromene-3-carboxylic acid were separated in separate experiments by eluting at room temperature with mobile phase (volume proportions) as follows:
11a: Acetonitrile-trifluoroacetic acid (100:0.1),
11b: Acetonitrile-trifluoroacetic acid (100:0.1),
11c: Acetonitrile-acetic acid (100:0.5), and
11d: Acetonitrile-acetic acid (100:0.25), respectively, at 1 mL/min. and detected with a photodiode array detector at 254 nm wavelength.

### Examples 12a and 12b

Using a column with 0.46 cm inner diameter and 25 cm length filled with CHIRALPAK® AS stationary phase, racemic mixtures of (R)- and (S)-6-ethyl-8-methyl-2-trifluoromethyl-2H-chromene-3-carboxylic acid were separated in separate experiments by eluting at room temperature with mobile phase (volume proportions) as follows:
12a: Heptane-2-propanol-trifluoroacetic acid (95:5:0.1), and
12b: Heptane-ethanol-trifluoroacetic acid (95:5:0.1), respectively, at 1 ml/min. and detected with a photodiode array detector at 280 nm wavelength.

### Example 13

Using a column with 0.46 cm inner diameter and 25 cm length filled with CHIRALCEL® OJ stationary phase, a racemic mixture of (R)- and (S)-6-ethyl-8-methyl-2-trifluoromethyl-2H-chromene-3-carboxylic acid was separated by eluting at room temperature with mobile phase Heptane-ethanol-trifluoroacetic acid (95:5:0.1) at 1 mL/min. and detected with a photodiode array detector at 280 nm wavelength.

### Examples 14a and 14b

Using a column with 0.46 cm inner diameter and 25 cm length filled with KROMASIL® DMB stationary phase, racemic mixtures of (R)- and (S)-6-ethyl-8-methyl-2-trifluoromethyl-2H-chromene-3-carboxylic acid were separated in separate experiments by eluting at room temperature with mobile phase (volume proportions) as follows:
14a: Heptane-2-propanol-trifluoroacetic acid (95:5:0.1), and
14b: Heptane-ethanol-trifluoroacetic acid (95:5:0.1), respectively, at 1 mL/min. and detected with a photodiode array detector at 254 nm wavelength.

### Examples 15a and 15b

Using a column with 0.46 cm inner diameter and 25 cm length filled with CHIRALPAK® AD stationary phase, racemic mixtures of (R)- and (S)-6-ethyl-8-methyl-2-trifluoromethyl-2H-chromene-3-carboxylic acid were separated in separate experiments by eluting at room temperature with mobile phase (volume proportions) as follows:
15a: Heptane-ethanol-trifluoroacetic acid (95:5:0.1), and
15b: Heptane-2-propanol-trifluoroacetic acid, respectively, at 1 mL/min. and detected with a photodiode array detector at 280 nm wavelength.

### Example 16

Using a column with 0.46 cm inner diameter and 25 cm length filled with CHIRALPAK® AS stationary phase, a racemic mixture of (R)- and (S)-6-chloro-5-methyl-2-trifluoromethyl-2H-chromene-3-carboxylic acid was separated by eluting at room temperature with mobile phase Heptane-ethanol-trifluoroacetic acid (95:5:0.1) at 1 mL/min. and detected with a photodiode array detector at 280 nm wavelength.

### Examples 17a and 17b

Using a column with 0.46 cm inner diameter and 25 cm length filled with CHIRALPAK® AD stationary phase, racemic mixtures of (R)- and (S)-8-ethyl-6-trifluoromethoxy-2-trifluoromethyl-2H-chromene-3-carboxylic acid were separated in separate experiments by eluting at room temperature with mobile phase (volume proportions) as follows:
17a: Heptane-2-propanol-trifluoroacetic acid (95:5:0.1), and
17b: Heptane-ethanol-trifluoroacetic acid (95:5:0.1), respectively, at 1 mL/min. and detected with a photodiode array detector at 254 nm wavelength.

### Example 18

Using a column with 0.46 cm inner diameter and 25 cm length filled with CHIRALCEL® OJ stationary phase, a racemic mixture of (R)- and (S)-8-ethyl-6-trifluoromethoxy-2-trifluoromethyl-2H-chromene-3-carboxylic acid was separated by eluting at room temperature with mobile phase Heptane-ethanol-trifluoroacetic acid (95:5:0.1) at 1 mL/min. and detected with a photodiode array detector at 254 nm wavelength.

### Examples 19a and 19b

Using a column with 0.46 cm inner diameter and 25 cm length filled with KROMASIL® DMB stationary phase, racemic mixtures of (R)- and (S)-8-ethyl-6-trifluoromethoxy-2-trifluoromethyl-2H-chromene-3-carboxylic acid were separated in separate experiments by eluting at room temperature with mobile phase (volume proportions) as follows:
14a: Heptane-2-propanol-trifluoroacetic acid (95:5:0.1), and

14b: Heptane-ethanol-trifluoroacetic acid (95:5:0.1), respectively, at 1 mL/min. and detected with a photodiode array detector at 280 nm wavelength.

TABLE 1
For each example, the retention times in minutes (“tR (min.)”)
for the first eluting peak (“Peak 1”) and the second eluting peak
(“Peak 2”), capacity factors for peak 1 (“k′₁”) and peak 2 (“k′₂”),
and selectivity factors for the more strongly retained Peak 2 versus
the less strongly retained Peak 1 (“α”) are shown.

| Example | Peak 1 | | Peak 2 | | |
|---|---|---|---|---|---|
| No. | tR (min.) | k′₁ | tR (min.) | k′₂ | α |
| 1a | 6.22 | 1.074 | 7.41 | 1.470 | 1.37 |
| 1b | 3.56 | 0.188 | 3.84 | 0.279 | 1.49 |
| 1c | 5.53 | 0.843 | 6.49 | 1.163 | 1.38 |
| 1d | 6.22 | 1.074 | 7.41 | 1.470 | 1.37 |
| 1e | 7.10 | 1.368 | 8.50 | 1.832 | 1.34 |
| 1f | 7.40 | 1.468 | 8.76 | 1.921 | 1.31 |
| 1g | 5.529 | 0.843 | 6.49 | 1.163 | 1.38 |
| 1h | 6.40 | 1.133 | 7.34 | 1.447 | 1.28 |
| 1i | 6.79 | 1.264 | 7.77 | 1.591 | 1.26 |
| 2a | 8.34 | 1.781 | 10.81 | 2.604 | 1.46 |
| 2b | 3.62 | 0.208 | 3.89 | 0.297 | 1.43 |
| 2c | 6.88 | 1.294 | 8.65 | 1.884 | 1.46 |
| 2d | 8.34 | 1.781 | 10.81 | 2.604 | 1.46 |
| 2e | 7.02 | 1.339 | 8.23 | 1.743 | 1.30 |
| 2f | 8.02 | 1.675 | 9.81 | 2.270 | 1.36 |
| 2g | 6.88 | 1.294 | 8.65 | 1.884 | 1.46 |
| 2h | 6.42 | 1.141 | 7.88 | 1.628 | 1.43 |
| 2i | 7.08 | 1.359 | 8.98 | 1.992 | 1.47 |
| 3a | 8.97 | 1.990 | 11.25 | 2.751 | 1.38 |
| 3b | 3.52 | 0.172 | 3.84 | 0.281 | 1.63 |
| 3c | 8.32 | 1.775 | 10.61 | 2.537 | 1.43 |
| 4 | 6.24 | 1.081 | 7.90 | 1.633 | 1.51 |
| 5a | 6.12 | 1.041 | 6.80 | 1.268 | 1.22 |
| 5b | 6.70 | 1.235 | 8.25 | 1.749 | 1.42 |
| 6a | 5.62 | 0.872 | 9.35 | 2.115 | 2.42 |
| 6b¹ | 6.42 | 1.139 | 9.91 | 2.304 | 2.02 |
| 6c² | 6.59 | 1.197 | 10.06 | 2.353 | 1.97 |
| 6d | 5.62 | 0.872 | 9.35 | 2.115 | 2.42 |
| 6e | 5.32 | 0.772 | 7.35 | 1.450 | 1.88 |
| 6f | 3.74 | 0.246 | 4.37 | 0.457 | 1.86 |
| 6g | 5.32 | 0.772 | 7.35 | 1.450 | 1.88 |
| 6h³ | 5.52 | 0.839 | 7.29 | 1.430 | 1.50 |
| 6i⁴ | 5.81 | 0.936 | 7.74 | 1.579 | 1.54 |
| 6j | 3.74 | 0.246 | 4.37 | 0.457 | 1.86 |
| 6k⁵ | 4.48 | 0.492 | 5.24 | 0.747 | 1.52 |
| 6l | 3.74 | 0.246 | 4.37 | 0.457 | 1.86 |
| 6m⁶ | 4.60 | 0.535 | 5.40 | 0.802 | 1.50 |
| 6n⁷ | 4.48 | 0.492 | 5.24 | 0.747 | 1.52 |
| 6o | 3.76 | 0.30 | 4.16 | 0.43 | 1.47 |
| 6p | 4.73 | 0.63 | 5.09 | 0.76 | 1.20 |
| 7a | 3.80 | 0.22 | 4.46 | 0.43 | 1.96 |
| 7b | 3.50 | 0.21 | 3.91 | 0.35 | 1.69 |
| 8a | 6.75 | 1.250 | 9.52 | 2.173 | 1.74 |
| 8b | 6.17 | N/a⁸ | 7.86 | N/a | 1.535 |
| 9 | 7.91 | 1.636 | 16.60 | 4.533 | 2.77 |
| 10 | 4.89 | 0.630 | 5.22 | 0.737 | 1.17 |
| 11a | 3.81 | 0.270 | 4.26 | 0.421 | 1.56 |
| 11b | 3.81 | 0.270 | 4.26 | 0.421 | 1.56 |
| 11c | 4.03 | 0.334 | 4.62 | 0.539 | 1.61 |
| 11d | 4.03 | 0.343 | 4.72 | 0.572 | 1.67 |
| 12a | 4.34 | 0.446 | 4.84 | 0.613 | 1.37 |
| 12b | 4.36 | 0.453 | 4.95 | 0.651 | 1.44 |
| 13 | 5.66 | 0.886 | 6.54 | 1.180 | 1.33 |
| 14a | 5.04 | 0.679 | 5.77 | 0.924 | 1.36 |
| 14b | 5.34 | 0.781 | 5.72 | 0.908 | 1.16 |
| 15a | 4.85 | 0.617 | 5.50 | 0.832 | 1.35 |
| 15b | 4.87 | 0.623 | 5.74 | 0.915 | 1.47 |
| 16 | 5.13 | 0.710 | 5.55 | 0.850 | 1.20 |
| 17a | 4.38 | 0.461 | 4.96 | 0.654 | 1.42 |
| 17b | 4.32 | 0.441 | 4.74 | 0.581 | 1.32 |
| 18 | 5.07 | 0.691 | 6.01 | 1.002 | 1.45 |
| 19a | 5.58 | 0.861 | 6.37 | 1.124 | 1.30 |
| 19b | 5.60 | 0.865 | 6.08 | 1.025 | 1.19 |

¹Area percent 60.4 (peak 1) and 39.6 (peak 2);
²Area percent 52.4 (peak 1) and 47.6 (peak 2);
³Area percent 48.1 (peak 1), 4.8 (peak 2), and 47.1 (peak 3), α is for peak 3 to peak 1;
⁴Area percent 35.6 (peak 1), 19.5 (peak 2), and 44.9 (peak 3), α is for peak 3 to peak 1;
⁵Area percent 51.2 (peak 1) and 48.8 (peak 2);
⁶Area percent 51.1 (peak 1) and 48.9 (peak 2);
⁷Area percent 51.2 (peak 1) and 48.8 (peak 2);
⁸N/a means not available;

### Example 20

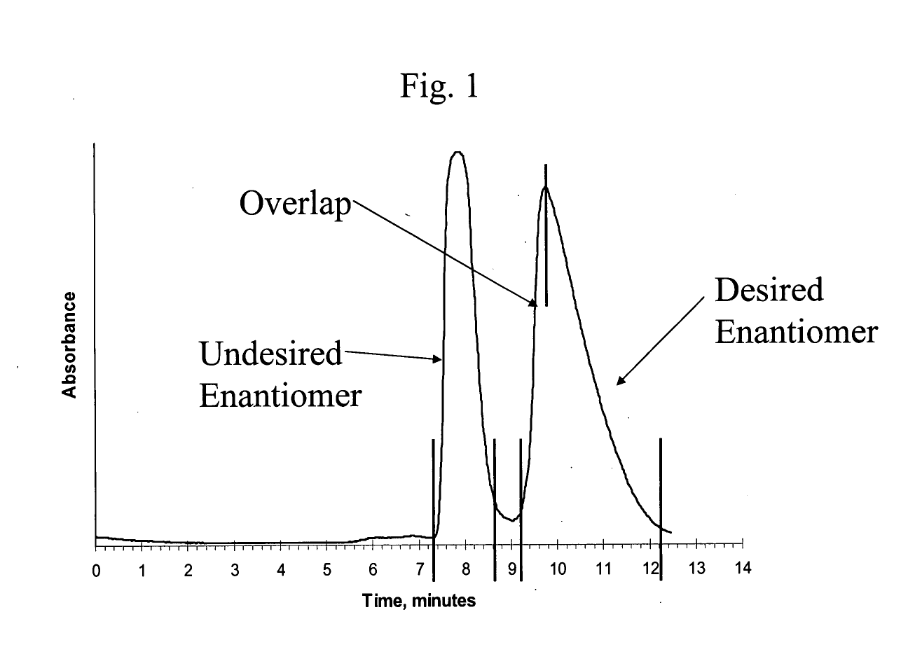

In an enantioselective batch preparative separation using a column with 10 cm inner diameter and 50 cm length filled with CHIRALPAK® AD (20 micron) stationary phase, 11 grams of a racemic mixture of (R)- and (S)-6-chloro-7-tert-butyl-2-trifluoromethyl-2H-chromene-3-carboxylic acid were separated by eluting at room temperature with mobile phase (volume proportions) Heptane-2-propanol-trifluoroacetic acid (90:10:0.5) at 500 mL/min. and detected with an UV detector at 254 nm wavelength to yield >90% of >98% e.e. (S)-6-chloro-7-tert-butyl-2-trifluoromethyl-2H-chromene-3-carboxylic acid. The productivity of this separation was 960 g racemate per kilogram of chiral stationary phase per day. A chromatogram of this separation is shown in FIG. 1.

### Example 21

In an enantioselective batch chromatography separation using a column with 50 mm inner diameter and 250 mm length filled with CHIRALPAK® AD (20 micron) stationary phase, a total of 190 mg of a racemic mixture of (R)- and (S)-6,8-dichloro-7-cyclohexylmethoxy-2-trifluoromethyl-2H-chromene-3-carboxylic acid was introduced in two batches of 95 mg each dissolved in 3 mL of mobile phase and eluted at room temperature with mobile phase (volume proportions) Heptane-2-propanol-trifluoroacetic acid (95:5:0.1) at a flow rate of 100 mL/min. and detected with an UV detector at 230 nm wavelength to yield 93 mg of a first eluting enantiomer at 100% ee and 90 mg of a second eluting enantiomer at 99.78% ee.

### Example 21a

In an enantioselective batch chromatography separation in single-column mode similar to that described in Example 21, using a column with 100 mm inner diameter and 500 mm length filled with CHIRALPAK® AS (20 micron) stationary phase, a total of 250 g of a racemic mixture of (R)- and (S)-6-chloro-5,7-dimethyl-2-trifluoromethyl-2H-chromene-3-carboxylic acid was introduced in batches of about 3 g each dissolved in 600 mL of mobile phase and cycle time of 4 minutes, and eluted at room temperature with mobile phase (volume proportions) acetonitrile-trifluoroacetic acid (100:0.1) at a flow rate of 500 mL/min. and separation productivity of 432 g of racemate per kg stationary phase per day and mobile phase consumption of 0.66 L per gram of racemate.

### Example 21b

In an enantioselective batch chromatography separation in single-column mode similar to that described in Example 21, using a column with 100 mm inner diameter and 500 mm length filled with CHIRALPAK® AD (20 micron) stationary phase, a total of 1200 g of a racemic mixture of (R)- and (S)-6-chloro-7-tert-butyl-2-trifluoromethyl-2H-chromene-3-carboxylic acid was introduced in batches of about 7 g each dissolved in 175 mL of mobile phase and cycle time of 7 minutes 15 seconds, and eluted at room temperature with mobile phase (volume proportions) heptane-isopropanol-trifluoroacetic acid (95:5:0.1) at a flow rate of 600 mL/min. and separation productivity of 590 g of racemate per kg stationary phase per day and mobile phase consumption of 0.62 L per gram of racemate.

### Example 21c

In an enantioselective batch chromatography separation in single-column mode similar to that described in Example 21, using a column with 100 mm inner diameter and 500 mm length filled with CHIRALPAK® AD (20 micron) stationary phase, a total of 2 kg of a racemic mixture of (R)- and (S)-6-chloro-7-tert-butyl-2-trifluoromethyl-2H-chromene-3-carboxylic acid was introduced in batches of about 9.4 g each dissolved in 230 mL of mobile phase and cycle time of 6 minutes, and eluted at room temperature with mobile phase (volume proportions) heptane-isopropanol-acetic acid (90:10:0.1) at a flow rate of 500 mL/min. and separation productivity of 960 g of racemate per kg stationary phase per day and mobile phase consumption of 0.32 L per gram of racemate to give (S)-6-chloro-7-tert-butyl-2-trifluoromethyl-2H-chromene-3-carboxylic acid in 93% yield (based on the calculated starting amount of (S) enantiomer).

### Example 22

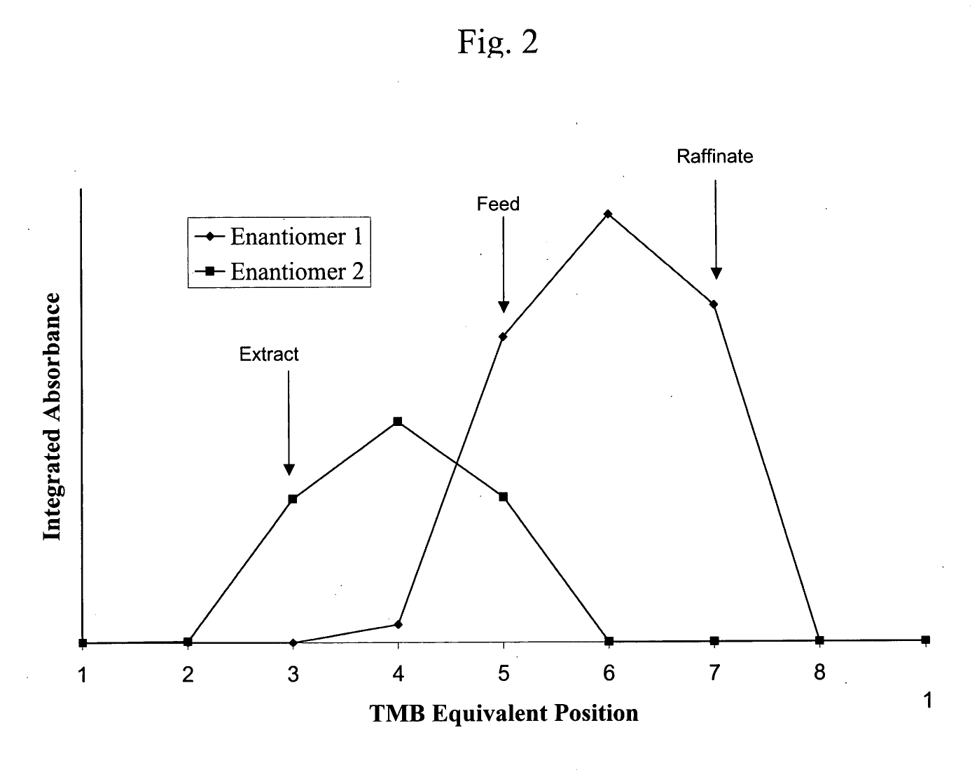

In an enantioselective SMB chromatography separation using eight (8) columns in a four zone, 2-2-2-2-2 arrangement, each column with 4.8 cm inner diameter and 11 cm length filled with CHIRALPAK® AD (20 micron) stationary phase, a racemic mixture of (R)- and (S)-6-chloro-7-tert-butyl-2-trifluoromethyl-2H-chromene-3-carboxylic acid was introduced at a concentration of 70 g/L of mobile phase, and eluted at 25° C. (the columns were jacketed and mobile phase was passed through a heat exchanger before entering the columns; the heat exchanger fluid was thermostated at 25° C. before entering the column jackets and heat exchanger) with mobile phase (volume proportions) Heptane-2-propanol-acetic acid (90:10:0.5) at the following final flow rates and switch time:
Flow rates:
   Feed: 22.34 mL/min.
   Eluent: 113.66 mL/min.
   Extract: 109.00 mL/min.
   Raffinate: 27.00 mL/min.
   Recycle: 225.00 mL/min.
   Zone Q1: 225.00 mL/min.
   Zone Q2: 116.00 mL/min.
   Zone Q3: 138.34 mL/min.
   Zone Q4: 111.34 mL/min.
Switch Time: 1.34 minutes
Operating pressure: between about 18 and about 20 bar;
   and detected by collecting samples from the pathway and analyzing the samples on an analytical enantioselective HPLC to yield (R)-6-chloro-7-tert-butyl-2-trifluoromethyl-2H-chromene-3-carboxylic acid in the raffinate stream with a purity of 99.98% and (S)-6-chloro-7-tert-butyl-2-trifluoromethyl-2H-chromene-3-carboxylic acid in the extract stream with a purity of 99.89% and 99.2% ee. An internal profile of this separation is shown in FIG. 2 as a plot of SMB eluent line position (“TMB Equivalent Position”) on the x-axis versus integrated absorbance on the y-axis. The internal profile was obtained by sampling the mobile phase at halfway through the switch time and measuring the internal concentrations of enantiomers offline. Since the samples for these internal concentration determinations were removed from between columns, the concentrations were equivalent to column positions 0.5, 1.5, 2.5, 3.5, 4.5, 5.5, 6.5, and 7.5. These positions are conventionally represented as TMB equivalent positions 1, 2, 3, 4, 5, 6, 7, and 8, respectively, in FIG. 2.

### Example 22a

In an enantioselective SMB chromatography separation using eight (8) columns in a four zone, 2-2-2-2-2 arrangement, each column with 4.8 cm inner diameter and 11 cm length filled with CHIRALPAK® AD (20 micron) stationary phase, a racemic mixture of (R)- and (S)-6-chloro-7-tert-butyl-2-trifluoromethyl-2H-chromene-3-carboxylic acid was introduced at a feed concentration of 72 g/L of mobile phase, and eluted at 25° C. with mobile phase (volume proportions) Heptane-2-propanol-acetic acid (90:10:0.5) at the following final flow rates and switch time:
Flow rates:
   Feed: 25.34 mL/min.
   Eluent: 113.66 mL/min.
   Extract: 109.0 mL/min.
   Raffinate: 30.0 ml/min.
   Recycle: 225.0 mL/min.
Switch Time: 1.34 minutes
Operating pressure: between about 18 bar;
   and detected by collecting samples from the pathway and analyzing the samples on an analytical enantioselective HPLC to yield (R)-6-chloro-7-tert-butyl-2-trifluoromethyl-2H-chromene-3-carboxylic acid in the raffinate stream with a purity of 100% and (S)-6-chloro-7-tert-butyl-2-trifluoromethyl-2H-chromene-3-carboxylic acid in the extract stream with a purity of 99.27% and >99% yield (based on a calculated starting amount of the (S)-enantiomer). The productivity of this separation was 3.04 kg racemate per kg stationary phase per day and mobile phase consumption was 0.078 L/g racemate.

### Example 23

In an enantioselective SMB chromatography separation using a Novasep Licosep Lab unit with eight (8) identical columns in a 2-2-2-2-2 arrangement, each column filled with 110 g of CHIRALPAK® AD stationary phase, a 328 L solution of a racemic mixture of (R)- and (S)-6-chloro-7-tert-butyl-2-trifluoromethyl-2H-chromene-3-carboxylic acid in mobile phase at a concentration of 34.8 g/L, prepared in a manner analogous to that described for the preparation of the feed solution in Example 24) was introduced continuously into the SMB unit, and the enantiomers were eluted at 25.0° C. with mobile phase (volume proportions) Heptane-ethanol-acetic acid (95:5:0.1) at the following final flow rates and switch time:
Flow rates:
   Feed: 45 mL/min.
   Eluent: 127.45 mL/min.
   Extract: 116.24 mL/min.
   Raffinate: 56.21 mL/min.
   Recycle: 301.80 mL/min.
Switch Time: 1.21 minutes.
   After five days the separation produced about 790 L of extract containing (S)-6-chloro-7-tert-butyl-2-trifluoromethyl-2H-chromene-3-carboxylic acid and 290 L of raffinate containing (R)-6-chloro-7-tert-butyl-2-trifluoromethyl-2H-chromene-3-carboxylic acid. After partial evaporation under reduced pressure (60 mbar) at 60° C. of the extract in a 100 L reactor protected from light to a volume of from about 8 L to about 10 L gave a precipitate of (S)-6-chloro-7-tert-butyl-2-trifluoromethyl-2H-chromene-3-carboxylic acid. The suspension was cooled to 5° C., filtered, and washed with mother liquor (4.5 L) followed by heptane (9 L), and dried under a nitrogen atmosphere overnight. The solids were transferred to a 20 L flask and dried on a rotary evaporator at 40-45° C. under full vacuum to yield (S)-6-chloro-7-tert-butyl-2-trifluoromethyl-2H-chromene-3-carboxylic acid with a purity of 99.7% and 98.6% ee in 36.8% yield (weight recovered enantiomer/weight racemic mixture). Work-up of the raffinate in a similar fashion yielded (R)-6-chloro-7-tert-butyl-2-trifluoromethyl-2H-chromene-3-carboxylic acid in 96.6% ee in 41.2% yield (weight recovered enantiomer/weight racemic mixture). The total recovery of separated enantiomers was 83.4%. The productivity of this separation was 2563 g racemate per kilogram of chiral stationary phase per day.

### Example 24

After two optimization runs, in an enantioselective SMB chromatography separation using a Novasep Licosep Lab unit with PEEK capillaries and eight (8) identical columns in a 2-2-2-2-2 arrangement, each column filled with 110 g of CHIRALCEL® OJ stationary phase, a 64 L solution of a 318.3 g mixture of 54.3% (R)- and 45.7% (S)-6-chloro-8-methyl-2-trifluoromethyl-2H-chromene-3-carboxylic acid in mobile phase at a concentration of 5.0 g/L, prepared by purging a suitable glass vessel with nitrogen gas, dissolving in a separate flask the mixture in ethanol with stirring, transferring the ethanol solution to the vessel, adding n-heptane to the solution in the vessel, adding a solution of acetic acid in n-heptane to the ethanol solution, and stirring the resulting solution for 45 minutes to give 64 L of a pale yellow solution of the mixture in Heptane-ethanol-acetic acid (95:5:0.1), was introduced continuously into the SMB unit, and the enantiomers were eluted at 25.0° C. with mobile phase (volume proportions) Heptane-ethanol-acetic acid (95:5:0.1) at the following final flow rates and switch time:
Flow rates:
   Feed: 15.0 mL/min.
   Eluent: 196.07 mL/min.
   Extract: 143.07 mL/min.
   Raffinate: 68.0 mL/min.
   Recycle: 450.0 mL/min.
Switch Time: 1.07 minutes.
   The raffinate stream contained (S)-6-chloro-8-methyl-2-trifluoromethyl-2H-chromene-3-carboxylic acid and the extract stream contained (R)-6-chloro-8-methyl-2-trifluoromethyl-2H-chromene-3-carboxylic acid. The raffinate stream was concentrated in a reactor under reduced pressure (600 mbar to 100 mbar) at 45 to 55° C. jacket temperature. The reactor was protected from light. The concentrate was transferred to a 20 L rotary evaporator, also protected from light, and further evaporated to dryness under reduced pressure (200 mbar to 18 mbar) in a 40° C. water bath to yield (S)-6-chloro-8-methyl-2-trifluoromethyl-2H-chromene-3-carboxylic acid. Work-up of the extract in a similar fashion yielded (R)-6-chloro-8-methyl-2-trifluoromethyl-2H-chromene-3-carboxylic acid. Combining raffinate streams from the second optimization procedure and this procedure gave (S)-6-chloro-8-methyl-2-trifluoromethyl-2H-chromene-3-carboxylic acid in 45.8% yield, 99.7% purity and >99.8% ee. Combining extract streams from the second optimization procedure and this procedure gave (R)-6-chloro-8-methyl-2-trifluoromethyl-2H-chromene-3-carboxylic acid in 65.4% yield, 99.8% purity and 95.8% ee. The productivity of this separation was 122.7 g mixture per kilogram of chiral stationary phase per day.

### Example 25

After two optimization runs, in an enantioselective SMB chromatography separation using a Novasep Licosep Lab unit and eight (8) identical columns in a 2-2-2-2-2 arrangement, each column filled with 110 g of CHIRALCEL® OJ stationary phase, 548 g mixture of 55.2% (R)- and 44.75% (S)-6,8-dimethyl-2-trifluoromethyl-2H-chromene-3-carboxylic acid in mobile phase at a concentration of 5.0 g/L to give 110 L of a pale yellow solution, prepared in a manner similar to the preparation of the solution described above in Example 24, was introduced continuously into the SMB unit, and the enantiomers were eluted at 18.0° C. with mobile phase (volume proportions) Heptane-ethanol-acetic acid (95:5:0.1) at the following final flow rates and switch time:
Flow rates:
   Feed: 14.97 mL/min.
   Eluent: 169.5 mL/min.
   Extract: 109.88 mL/min.
   Raffinate: 74.59 mL/min.
   Recycle: 455.0 mL/min.
Switch Time: 0.88 minutes.
   The extract stream contained (S)-6,8-dimethyl-2-trifluoromethyl-2H-chromene-3-carboxylic acid and the raffinate stream contained (R)-6,8-dimethyl-2-trifluoromethyl-2H-chromene-3-carboxylic acid. Combining extract streams from the second optimization procedure and this procedure gave (S)-6,8-dimethyl-2-trifluoromethyl-2H-chromene-3-carboxylic acid in 99.0% purity and 99.0% ee. Combining raffinate streams from the third optimization procedure and this procedure gave (R)-6,8-dimethyl-2-trifluoromethyl-2H-chromene-3-carboxylic acid in 99.9% purity and 87.6% ee. The productivity of this separation was 127.4 g mixture per kilogram of chiral stationary phase per day.

### Example 26

After three optimization runs, an enantioselective SMB chromatography separation using a Novasep Licosep Lab unit and eight (8) identical columns in a 2-2-2-2-2 arrangement, each column filled with 110 g of CHIRALPAK® AD stationary phase, a feed solution of a mixture of (R)- and (S)-8-ethyl-6-trifluoromethoxy-2-trifluoromethyl-2H-chromene-3-carboxylic acid in mobile phase at a concentration of 26.0 g/L, prepared in a manner similar to the preparation of the solution described above in Example 24, in Heptane-ethanol-trifluoroacetic acid (98:2:0.1), was introduced continuously into the SMB unit, and the enantiomers were eluted at a measured temperature of 22.4° C. (set to 25° C.) with mobile phase (volume proportions) Heptane-ethanol-trifluoroacetic acid (98:2:0.1) at the following final flow rates and switch time:
Flow rates:
   Feed: 20 mL/min.
   Eluent: 140 mL/min.
   Extract: 47 mL/min.
   Raffinate: 113 mL/min.
   Recycle: 437.5 mL/min.
Switch Time: 0.76 minutes.
   The extract stream contained (S)-8-ethyl-6-trifluoromethoxy-2-trifluoromethyl-2H-chromene-3-carboxylic acid and the raffinate stream contained (R)-8-ethyl-6-trifluoromethoxy-2-trifluoromethyl-2H-chromene-3-carboxylic acid. Combining extract streams from the third optimization procedure and this procedure gave (S)-8-ethyl-6-trifluoromethoxy-2-trifluoromethyl-2H-chromene-3-carboxylic acid in 36.8% yield and 98.6% ee after crystallization. Combining raffinate streams from the third optimization procedure and this procedure gave (R)-8-ethyl-6-trifluoromethoxy-2-trifluoromethyl-2H-chromene-3-carboxylic acid in 41.2% yield and 96.6% ee after crystallization. The productivity of this separation was 850.91 g mixture per kilogram of chiral stationary phase per day.

### Example 27

In an enantioselective SMB chromatography separation using a Novasep Licosep Lab unit with eight (8) identical columns in a 2-2-2-2-2 arrangement, each column filled with 110 g of CHIRALPAK® AD stationary phase, a 116.1 L solution containing 582 grams of a racemic mixture of (R)- and (S)-6-chloro-5,7-dimethyl-2-trifluoromethyl-2H-chromene-3-carboxylic acid in mobile phase at a concentration of 4.728 g/L (prepared by charging a 20 L flask with the (R)- and (S)-6-chloro-5,7-dimethyl-2-trifluoromethyl-2H-chromene-3-carboxylic acid, adding acetonitrile (116 L) in 10 L portions to a suspension thereof, then adding trifluoroacetic acid (116 mL), stirring for 1 hour, and filtering the resulting solution through a filter Polycap HD 75 to a second vessel) was introduced continuously into the SMB unit, and the enantiomers were eluted at 26-27° C. with mobile phase (volume proportions) acetonitrile-trifluoroacetic acid (99.9:0.1, Positions 4 and 5) at the following final flow rates and switch time:
Flow rates:
   Feed: 11.7 mL/min.
   Eluent: 137 mL/min.
   Extract: 96.8 mL/min.
   Raffinate: 51.9 mL/min.
   Recycle: 440 mL/min.
Switch Time: 0.49 minutes.
   About 70% of the material was separated to give in-specification (S)-6-chloro-5,7-dimethyl-2-trifluoromethyl-2H-chromene-3-carboxylic acid.

In-specification characterizations of (R)- and (S)-6-chloro-5,7-dimethyl-2-trifluoromethyl-2H-chromene-3-carboxylic acid were determined by enantioselective HPLC as follows:
HPLC Method 1: column CHIRALPAK® AD chiral stationary phase; 250 mm×4.6 mm column dimensions; 20 μm diameter stationary phase particle size; mobile phase flow rate 1.0 mL/minute; 10 μL injection volume; 20° C. operating temperature; detection at 254 nm; mobile phase acetonitrile-trifluoroacetic acid 100:0.1 (volume:volume); sample protected from sunlight; run stop time 8 minutes; retention time of 3.63 minutes for (S)-enantiomer and 4.27 minutes for (R) enantiomer. HPLC Method 2: column CHIRALPAK® AD chiral stationary phase; 250 mm×4.6 mm column dimensions; 10 μm diameter stationary phase particle size; mobile phase flow rate 1.0 mL/minute; 10 μL injection volume; ambient operating temperature; detection at 254 nm; mobile phase acetonitrile-trifluoroacetic acid 100:0.1 (volume:volume); sample protected from sunlight; run stop time 15 minutes; retention time of 3.64 minutes for (S)-enantiomer and 4.30 minutes for (R) enantiomer.

The 30% of the material that was not in-specification was rotary evaporated to give 181 g. This material was used to prepare a second feed solution in acetonitrile (36 L) and trifluoroacetic acid (36 mL) in a manner similar to that described above except that 5 L portions of acetonitrile were used. The concentration of the second feed solution was estimated to be 4.7 g/L. The second feed solution was introduced continuously into a SMB unit that was set up as before, and the enantiomers were eluted at 22.5° C. (as setting) with mobile phase (volume proportions) acetonitrile-trifluoroacetic acid (99.9:0.1, Positions 4 and 5) at the above final flow rates and switch time to give additional separated material in the raffinate and extract streams.

The two raffinate streams containing (S)-6-chloro-5,7-dimethyl-2-trifluoromethyl-2H-chromene-3-carboxylic acid were combined. The two extract streams containing (R)-6-chloro-5,7-dimethyl-2-trifluoromethyl-2H-chromene-3-carboxylic acid were combined.

The combined raffinate was rotary evaporated to dryness in a jacket at 45° C. to give 237.4 of (S)-6-chloro-5,7-dimethyl-2-trifluoromethyl-2H-chromene-3-carboxylic acid that was 99.64% enantiomerically pure (i.e., 99.28% ee). While the organic purity was 100%, due to technical problems a content of approximately 600 parts per million (“ppm”) of iron, 120 ppm of nickel, and 120 ppm of chromium was found. Dissolving 206.4 g of the contaminated material in 0.8 L dichloromethane, and purifying the mixture by chromatography on 3.55 kg of silica gel using dichloromethane as eluent yielded purified (S)-6-chloro-5,7-dimethyl-2-trifluoromethyl-2H-chromene-3-carboxylic acid. Fractions containing product were transferred to a rotary evaporator through an Inline filter, and rotary evaporated under reduced pressure at jacket temperature 45° C. to give 174 g. This material was transferred to a 1.0 L reaction vessel, methylcyclohexane (578 mL) was added at 20° C., and the resulting suspension was heated at 110° C. to reflux. The mixture was cooled over 62 minutes to an internal temperature of 2° C., the resulting suspension was stirred at 3° C. for 3.5 hours, and filtered. The filter cake was washed with methylcyclohexane, dried briefly with nitrogen on the filter, and rotary dried at reduced pressure and jacket temperature of 45° C. over 18 hours to give 160 g (78% recovery) of (S)-6-chloro-5,7-dimethyl-2-trifluoromethyl-2H-chromene-3-carboxylic acid as a yellowish free-flowing crystalline solid (1 ppm Aluminum, 1 ppm chromium, 3 ppm copper, 3 ppm iron, <1 ppm Molybdenum, 1 ppm nickel, and <1 ppm vanadium).

The combined extract was rotary evaporated to dryness in a jacket at 45° C. to give 268 of (R)-6-chloro-5,7-dimethyl-2-trifluoromethyl-2H-chromene-3-carboxylic acid that was 97.52% enantiomerically pure (i.e., 95.04% ee).

The total recovery of separated enantiomers was 86.8%. The productivity of this separation was 90.56 g racemate per kilogram of chiral stationary phase per day.

### Example 28

In an enantioselective steady state recycling chromatography separation in single-column mode as described in U.S. Pat. No. 6,063,284, using a column with 50 mm inner diameter and 500 mm length that is filled with CHIRALPAK® AD (20 micron) stationary phase, a total of 278.06 g of a racemic mixture of (R)- and (S)-6,8-dimethyl-2-trifluoromethyl-2H-chromene-3-carboxylic acid was introduced by loading of a solution of 1.4 g of the chromene dissolved in 35 mL of mobile phase during each cycle of a multicycle process, and the enantiomers were eluted at room temperature with mobile phase (volume proportions) Heptane-ethanol-trifluoroacetic acid (95:5:0.1). The mobile phase was pumped at a flow rate of 150 mL/min. and a cycle time of 10 minutes 30 seconds. Each injection of the chromene solutions occurred at 4 minutes 39 seconds into each cycle. Separated enantiomers were detected with an UV detector at 375 nm wavelength. A first enantiomer was collected between 33 seconds and 2 minutes 48 seconds and a second enantiomer was collected between 7 minutes 3 seconds and 9 minutes 40 seconds of each cycle to yield a total of 135.42 g of the first eluting enantiomer at 100% ee and a total of 134.50 g of the second eluting enantiomer at 100% e.e., wherein % e.e. was determined by analytical enantioselective chromatography according to the method of the present invention. The separation productivity was 326 g of racemate per kg stationary phase per day.

### Example 28a

In an enantioselective steady state recycling chromatography separation in single-column mode similar to that described in Example 28, using a column with 50 mm inner diameter and 500 mm length filled with CHIRALCEL® OJ (20 micron) stationary phase, a total of 290 g of a racemic mixture of (R)- and (S)-8-ethyl-6-trifluoromethoxy-2-trifluoromethyl-2H-chromene-3-carboxylic acid was introduced in batches of about 0.94 g each dissolved in 35 m]L of mobile phase and cycle time of 18 minutes, and eluted at room temperature with mobile phase (volume proportions) heptane-ethanol-trifluoroacetic acid (95:5:0.1) at a flow rate of 100 mL/min. and separation productivity of 128 g of racemate per kg stationary phase per day.

### Example 29

A two-column mode steady state recycling chromatography separation as illustrated in FIG. 2 of U.S. Pat. No. 5,630,943 and described for Example 1 of U.S. Pat. No. 5,630,943, uses a first column and a second column each with 50 mm inner diameter and 500 mm length and filled with CHIRALPAK® AD (20 micron) stationary phase. A total of 278.06 g of a racemic mixture of (R)- and (S)-6,8-dimethyl-2-trifluoromethyl-2H-chromene-3-carboxylic acid is introduced onto each column by loading solutions of 1.4 g of the chromene dissolved in 35 mL of mobile phase during each cycle of a multicycle process, and the enantiomers are eluted at room temperature with mobile phase (volume proportions) Heptane-ethanol-trifluoroacetic acid (95:5:0.1). The mobile phase is pumped at a flow rate of 150 mL/min. from Pump 1 and Pump 2, when Pump 2 is used, and a per column cycle time of 10 minutes 30 seconds. Each injection of a chromene solution occurs at 4 minutes 39 seconds into each per column cycle time. Separated enantiomers are detected with an UV detector at 375 nm wavelength. A first enantiomer is collected between 33 seconds and 2 minutes 48 seconds and a second enantiomer is collected between 7 minutes 3 seconds and 9 minutes 40 seconds of each per column cycle time to yield an expected total of 135.42 g of the first eluting enantiomer at 100% ee and an expected total of 134.50 g of the second eluting enantiomer at 100% e.e., wherein % e.e. is determined by analytical enantioselective chromatography according to the method of the present invention.

### Example 30

Using a column with 0.46 cm inner diameter and 25 cm length filled with CYCLOBOND I 2000 stationary phase, a racemic mixture of (R)- and (S)-8-chloro-6-methoxy-2-trifluoromethyl-2H-chromene-3-carboxylic acid, sodium salt was separated by eluting at room temperature with mobile phase 0.05 M sodium biphosphate (adjusted to pH 9.0 with 0.1 M sodium hydroxide)-acetonitrile (50:50 v/v) at a flow rate of 1 mL/min. and detected with a photodiode array detector at 210 nm wavelength.

### Example 31

Using a column with 0.46 cm inner diameter and 25 cm length filled with CHIRALPAK® AS stationary phase, a racemic mixture of (R)- and (S)-6-chloro-2-trifluoromethyl-1,2-dihydro-quinoline-3-carboxylic acid is separated by eluting at room temperature with Methanol (100%), at 1 mL/min. and detecting the (S)-enantiomer with a photodiode array detector at 254 nm wavelength.

### Example 32

In Polar Organic mode, an enantioselective HPLC separation using a column with 0.46 cm inner diameter and 25 cm length filled with CHIRALPAK® AD stationary phase, a racemic mixture of (R)- and (S)-6,8-dimethyl-2-trifluoromethyl-2H-chromene-3-carboxylic acid was separated by eluting at room temperature with mobile phase (volume proportions) methanol-trifluoroacetic acid (100:0.1) at a flow rate of 1 mL/minute and a UV detector at 254 nm to give a separation of enantiomers with α=1.83.

### Examples 33a and 33b

In an enantioselective HPLC separation using a column with 0.46 cm inner diameter and 25 cm length filled with CHIRALPAK® AS stationary phase, a racemic mixture of (R)- and (S)-6-chloro-5,7-dimethyl-2-trifluoromethyl-2H-chromene-3-carboxylic acid ethyl ester was separated in two separate experiments by eluting at room temperature with the following mobile phases (volume proportions):
33a: methanol (100);
33b: ethanol (100);
   at flow rates of 1 mL/minute and using an UV detector at 254 nm to give a separation of enantiomers with the following characteristics:
33a: tR (min.) for the first eluting peak about 3.8 minutes and the second eluting peak about 4.4 minutes, α=1.67;
33b: tR (min.) for the first eluting peak 3.58 minutes and the second eluting peak 3.96 minutes, capacity factors k′₁ of 0.22 and k′₂ of 0.36, and α=1.62.

### Example 34

In an enantioselective HPLC separation using a column with 0.46 cm inner diameter and 25 cm length filled with CHIRALPAK® AS stationary phase, a racemic mixture of (R)- and (S)-6-trifluoromethoxy-8-ethyl-2-trifluoromethyl-2H-chromene-3-carboxylic acid ethyl ester was separated by eluting at room temperature with mobile phase (volume proportions) methanol (100) at a flow rate of 1 mL/minute and using an UV detector at 254 nm to give a separation of enantiomers with α=1.68.

### Example 35

In an enantioselective HPLC separation using a column with 0.46 cm inner diameter and 25 cm length filled with CHIRALPAK® AS stationary phase, a racemic mixture of (R)- and (S)-6,8-dimethyl-2-trifluoromethyl-2H-chromene-3-carboxylic acid ethyl ester was separated by eluting at room temperature with mobile phase (volume proportions) methanol (100) at a flow rate of 1 mL/minute and using an UV detector at 254 nm to give a separation of enantiomers with tR (min.) for the first eluting peak about 3.6 minutes and the second eluting peak about 4.0 minutes.

### Example 36

In an enantioselective HPLC separation using a column with 0.46 cm inner diameter and 25 cm length filled with CHIRALPAK® AS stationary phase, a racemic mixture of (R)- and (S)-6-chloro-7-tert-butyl-2-trifluoromethyl-2H-chromene-3-carboxylic acid ethyl ester was separated by eluting at room temperature with mobile phase (volume proportions) methanol (100) at a flow rate of 1 mL/minute and using an UV detector at 254 nm to give a separation of enantiomers with tR (min.) for the first eluting peak about 3.8 minutes and the second eluting peak about 4.4 minutes.

### Example 37

In an enantioselective fractional crystallization of (R)- and (S)-6-chloro-7-tert-butyl-2-trifluoromethyl-2H-chromene-3-carboxylic acid (“R/S-CTBTCCA”), the R/S-CTBTCCA is combined with 1.0 mole equivalents (to R/S-CTBTCCA) of (+)-cinchonine, and the mixture is dissolved in 10 mL of methylene chloride per gram of R/S-CTBTCCA. About 30 mL of heptane-methyl tert-butyl ether (1:3 volume:volume, methyl tert-butyl ether is “MTBE”) per gram of R/S-CTBTCCA is added. The total volume of the resulting mixture is reduced by vacuum distillation to about 10 mL of mixture per gram of R/S-CTBTCCA. A white slurry is formed. About 30 mL of heptane-MTBE (1:3, volume:volume) per gram of R/S-CTBTCCA is added, and then the volume of the mixture is reduced by vacuum distillation to about 12 mL of mixture per gram of R/S-CTBTCCA. About 24 mL of MTBE per gram of R/S-CTBTCCA is added to yield a concentration of about 1-gram of R/S-CTBTCCA per 36 mL of heptane-MTBE (1:3, volume:volume). The resultant slurry is cooled to 0-5° C. and filtered. The filter cake, which contains (R)-6-chloro-7-tert-butyl-2-trifluoromethyl-2H-chromene-3-carboxylic acid (+)-cinchonine salt, is rinsed with additional MTBE. The first filtrate and first rinsate, which each contain (S)-6-chloro-7-tert-butyl-2-trifluoromethyl-2H-chromene-3-carboxylic acid (+)-cinchonine salt, are combined.

To the combined first filtrate and first rinsate is added 6 m]L water per gram of R/S-CTBTCCA followed by the slow addition of 4 mL of 1N HCl per gram of R/S-CTBTCCA to form a two-phase mixture. The mixture is stirred and the phases are separated. To the resulting yellow organic phase is added 2 mL 1N HCl per gram of R/S-CTBTCCA and 6 mL of water per gram of R/S-CTBTCCA, the resulting mixture is stirred, and the phases are separated. To the organic phase is added 10 mL of water per gram of R/S-CTBTCCA, the resulting mixture is stirred, and phases are separated. A 0.5 mL aliquot is removed from the organic phase and rotary evaporated to dryness, and the total mass of (S)-6-chloro-7-tert-butyl-2-trifluoromethyl-2H-chromene-3-carboxylic acid in the organic phase is calculated. The volume of the organic phase is reduced by vacuum distillation to a final volume of about 4 mL of mixture per calculated gram of (S)-6-chloro-7-tert-butyl-2-trifluoromethyl-2H-chromene-3-carboxylic acid. An off-white slurry forms during this vacuum distillation. About 0.16 mL of ethyl acetate (“EtOAc”) per gram of (S)-6-chloro-7-tert-butyl-2-trifluoromethyl-2H-chromene-3-carboxylic acid is added to yield an EtOAc-heptane (4:96, volume:volume) solvent system (the MTBE is removed in the last distillation). This slurry is stirred for about 1 hour, cooled to 0-5° C., stirred for an additional hour, and filtered. The filter cake is rinsed with heptane and the second filtrate and second rinsate are combined.

The combined second filtrate and second rinsate is concentrated to a volume of about 2 mL of mixture per gram of (S)-6-chloro-7-tert-butyl-2-trifluoromethyl-2H-chromene-3-carboxylic acid. The mixture is cooled to 0-5° C., stirred for about 1 hour, and filtered. The filter cake is rinsed with heptane and dried in a vacuum oven at 50° C. under house vacuum (typically from about 25 to about 28 mm Hg) with a nitrogen gas sweep. This process provides greater than 99% enantiomerically pure (i.e., greater than 98% ee) (S)-6-chloro-7-tert-butyl-2-trifluoromethyl-2H-chromene-3-carboxylic acid with an expected yield of 30% from R/S-CTBTCCA (calculated by dividing the amount of separated enantiomer by the starting amount of R/S-CTBTCCA).

### Example 38

In an enantioselective fractional crystallization of (R)- and (S)-8-ethyl-6-trifluoromethoxy-2-trifluoromethyl-2H-chromene-3-carboxylic acid (“R/S-ETTCCA”), R/S-ETTCCA is combined with 1.0 mole equivalents of (S)-(−)-N-benzyl-α-methylbenzylamine (“BAMBA”), and the mixture is dissolved in 10 mL of MTBE per gram of R/S-ETTCCA. About 20 mL of heptane per gram of R/S-ETTCCA is added, and the volume of the resulting mixture is reduced by vacuum distillation to about 5 mL of mixture per gram of R/S-ETTCCA. About 5 mL of heptane per gram of R/S-ETTCCA is added. A white slurry forms. The slurry is stirred for 4 hours at 20° C., filtered, and the resulting filter cake is rinsed with heptane. The crystalline filter cake is dried in a vacuum oven at 50° C. under house vacuum (typically from about 25 to about 28 mm Hg) with a nitrogen gas sweep. To improve the enantiomeric purity, the filter cake is reslurried in about 10 mL of heptane per gram of filter cake at 20° C. for about 4 hours. The solids are filtered, and the resulting filter cake is rinsed with heptane and dried in a vacuum oven at 50° C. with a nitrogen gas sweep. This process provides greater than 99% enantiomerically pure (i.e., >98% e.e.) (S)-8-ethyl-6-trifluoromethoxy-2-trifluoromethyl-2H-chromene-3-carboxylic acid (S)-(−)-N-benzyl-α-methylbenzylamine salt with an expected yield of 37% from the racemate (calculated by dividing the amount of separated enantiomer by the starting amount of R/S-ETTCCA). In a manner similar to that described above for the conversion of (S)-6-chloro-7-tert-butyl-2-trifluoromethyl-2H-chromene-3-carboxylic acid (+)-cinchonine salt to (S)-6-chloro-7-tert-butyl-2-trifluoromethyl-2H-chromene-3-carboxylic acid, the (S)-8-ethyl-6-trifluoromethoxy-2-trifluoromethyl-2H-chromene-3-carboxylic acid (S)-(−)-N-benzyl-α-methylbenzylamine salt is converted to (S)-8-ethyl-6-trifluoromethoxy-2-trifluoromethyl-2H-chromene-3-carboxylic acid in high yield and enantiomeric purity.

### Example 39

In an enantioselective fractional crystallization of (R)- and (S)-6,8-dimethyl-2-trifluoromethyl-2H-chromene-3-carboxylic acid (“R/S-DTCCA”), R/S-DTCCA is combined with 1.2 mole equivalents of (S)-(−)-N-benzyl-α-methylbenzylamine, and the mixture is combined with 10 mL of MTBE per gram of R/S-DTCCA. The mixture is heated to 50° C. to allow for complete dissolution, and the resulting solution is cooled to 20° C. To the solution is added 10 mL heptane per gram of mixture and optionally seed crystals of (S)-6,8-dimethyl-2-trifluoromethyl-2H-chromene-3-carboxylic acid, and the resulting slurry is stirred for about 16 hours. The slurry is filtered, and the resulting crystalline filter cake is rinsed with heptane and dried in a vacuum oven at 50° C. under house vacuum (typically from about 25 to about 28 mm Hg) with nitrogen gas sweep. To improve the enantiomeric purity, the crystals are slurried in 10 mL of MTBE per gram of isolated crystals, and the mixture is heated to about 40° C. to form a thin slurry. The mixture is cooled to about 20° C., and 10 mL of heptane pre gram of isolated crystals is added. The resulting slurry is stirred for about 16 hours, filtered, and the resulting filter cake is rinsed with heptane. The crystalline filter cake is dried under vacuum in a vacuum oven at 50° C. under house vacuum (typically from about 25 to about 28 mm Hg) with a nitrogen gas sweep. This process provides 97% enantiomerically pure (i.e., 94% ee) (S)-6,8-dimethyl-2-trifluoromethyl-2H-chromene-3-carboxylic acid (S)-(−)-N-benzyl-α-methylbenzylamine salt with an expected yield of 38% from R/S-DTCCA (calculated by dividing the amount of separated enantiomer by the starting amount of R/S-DTCCA) In a manner similar to that described above for the conversion of (S)-6-chloro-7-tert-butyl-2-trifluoromethyl-2H-chromene-3-carboxylic acid (+)-cinchonine salt to (S)-6-chloro-7-tert-butyl-2-trifluoromethyl-2H-chromene-3-carboxylic acid, the (S)-6,8-dimethyl-2-trifluoromethyl-2H-chromene-3-carboxylic acid (S)-(−)-N-benzyl-α-methylbenzylamine salt is converted to (S)-6,8-dimethyl-2-trifluoromethyl-2H-chromene-3-carboxylic acid in high yield and enantiomeric purity.

### Example 40

An enantioselective fractional crystallization of (R)- and (S)-6-chloro-8-methyl-2-trifluoromethyl-2H-chromene-3-carboxylic acid (“R/S-CMTCCA”) is carried out in a manner similar to that described above in for the resolution of R/S-DTCCA to provide 99% enantiomerically pure (i.e., 98% e.e.) (S)-6-chloro-8-methyl-2-trifluoromethyl-2H-chromene-3-carboxylic acid (S)-(−)-N-benzyl-α-methylbenzylamine salt with an expected yield of 34% from R/S-CMTCCA (calculated by dividing the amount of separated enantiomer by the starting amount of R/S-CMTCCA). In a manner similar to that described above for the conversion of (S)-6-chloro-7-tert-butyl-2-trifluoromethyl-2H-chromene-3-carboxylic acid (+)-cinchonine salt to (S)-6-chloro-7-tert-butyl-2-trifluoromethyl-2H-chromene-3-carboxylic acid, the (S)-6-chloro-8-methyl-2-trifluoromethyl-2H-chromene-3-carboxylic acid (S)-(−)-N-benzyl-α-methylbenzylamine salt is converted to (S)-6-chloro-8-methyl-2-trifluoromethyl-2H-chromene-3-carboxylic acid in high yield and enantiomeric purity.

### Example 41

In an enantioselective fractional crystallization of (R)- and (S)-6-chloro-5,7-dimethyl-2-trifluoromethyl-2H-chromene-3-carboxylic acid (“R/S-CDTCCA”), R/S-CDTCCA is combined with 1.0 mole equivalents of (+)-cinchonine, and the mixture is dissolved in 10 mL of methylene chloride-MTBE (1:1, volume:volume) per gram of R/S-CDTCCA. Using a nitrogen gas sweep, the volume of the solution is reduced until an oil forms. About 20 mL of MTBE per gram of R/S-CDTCCA is added, and the volume of the mixture is reduced to give an oil. Another 20 mL of MTBE per gram of R/S-CDTCCA is added, and the volume of the mixture is reduced to give an oil. Then about 20 mL of heptane and about 10 mL MTBE each per gram of R/S-CDTCCA is added to form a slurry. The resulting slurry is stirred for about 16 hours, and then filtered into a filter funnel. The resulting crystalline filter cake is dried by applying a vacuum to the filter funnel. The filter cake is slurried in about 20 mL of ethanol-MTBE (25:75, volume:volume), and the mixture is stirred for about 4 hours, filtered, and the resulting filter cake is rinsed with ethanol-MTBE (25:75, volume:volume). The crystalline filter cake is dried in a vacuum oven at 50° C. under house vacuum (typically from about 25 to about 28 mm Hg) with a nitrogen gas sweep. The solids are reslurried in about 20 mL of ethanol-MTBE (25:75, volume:volume), and the mixture is stirred for about 16 hours. The slurry is filtered and the resulting filter cake is rinsed with ethanol-MTBE (25:75, volume:volume). The solids are dried in a vacuum oven under house vacuum (typically from about 25 to about 28 mm Hg) with a nitrogen gas sweep. This process provides 98% enantiomerically pure (i.e., 96% e.e.) (S)-6-chloro-5,7-dimethyl-2-trifluoromethyl-2H-chromene-3-carboxylic acid (+)-cinchonine salt with an expected yield of 24% from R/S-CDTCCA (calculated by dividing the amount of separated enantiomer by the starting amount of R/S-CDTCCA). In a manner similar to that described above for the conversion of (S)-6-chloro-7-tert-butyl-2-trifluoromethyl-2H-chromene-3-carboxylic acid (+)-cinchonine salt to (S)-6-chloro-7-tert-butyl-2-trifluoromethyl-2H-chromene-3-carboxylic acid, the (S)-6-chloro-5,7-dimethyl-2-trifluoromethyl-2H-chromene-3-carboxylic acid (+)-cinchonine salt is converted to (S)-6-chloro-5,7-dimethyl-2-trifluoromethyl-2H-chromene-3-carboxylic acid in high yield and enantiomeric purity.

### Example 42

To a 500 mL, single-necked round bottom flask marked with a 50 mL volume line was added 5.0 g (14.9 mmol) of (R)- and (S)-6-chloro-7-tert-butyl-2-trifluoromethyl-2H-chromene-3-carboxylic acid, 4.4 g (14.9 mmol) of (+)-cinchonine, and 50 mL of CH₂Cl₂, and the mixture was stirred with a magnetic stir bar until a solution formed. To the solution was added 150 mL of Isopar C-MTBE (25:75, volume:volume), and the resulting mixture was slowly distilled on a rotary evaporator down to a volume of 50 mL. To the mixture was added 150 mL of octane-MTBE (25:75, volume:volume), and a slurry formed. The resulting mixture was distilled down to about 60 mL to remove all residual CH₂Cl₂. To the resulting mixture was added 120 mL of MTBE, and the mixture was stirred with a magnetic stir bar for 16 hours at room temperature. A thick slurry had formed, which was cooled to 0-5° C. and stirred for 1 hour. The resulting paste-like solids were filtered at 0.0° C., and the first filter cake was washed with 5 mL of MTBE. The first wash was added to the first mother liquor. The first filter cake was dried in a vacuum oven at 55° C. under house vacuum with a nitrogen sweep to give 4.834 g of dry solids that contained a 94.56:5.44 (area/area) ratio of the (R)-enantiomer to the (S)-enantiomer by HPLC. (HPLC procedure: Diluted a sample with heptane-ethanol (90:10, volume:volume) to final concentration of 0.5 mg/mL, and 10 μL were introduced onto a Column: CHIRALPAK® AD (4.6 mm inner diameter×250 mm length), and eluted with mobile phase comprising heptane-ethanol-trifluoroacetic acid (95:5:0.1, volume proportions) at a flow rate at 1 mL/minute for 9 minutes. The enantiomers were detected with a UV detector at 254 nm.)

The combined first wash and first mother liquor were added to a 500 mL, single-necked round bottom flask marked with a 50 mL volume line, and the mixture was concentrated to a volume of 50 mL. To the concentrate was added 30 mL of H₂O followed by a slow addition of 20 mL of 1N HCl. The mixture was stirred, and the phases were separated. The resulting yellow organic phase was washed with a combination of 10 mL of 1N HCl and 30 mL of water. The mixture was stirred, and the phases were separated. The resulting organic phase was washed with 50 mL of water.

A 0.5 mL aliquot of the organic phase was dried in a vacuum oven at 55° C., and the amount of free 6-chloro-7-tert-butyl-2-trifluoromethyl-2H-chromene-3-carboxylic acid in the organic phase was determined to be 0.0218 g per 0.5 mL and 3.052 g in 70 mL of organic phase. The organic phase was distilled down in a 250 mL, 1-necked round bottom flask to a final volume of 12.2 mL. To the concentrate was added 0.49 mL of ethyl acetate (“EtOAc”) to give an estimated solvent composition of Isopar C-EtOAc (96:4, volume:volume). The resulting mixture was stirred for one hour, then cooled to 0-5° C. and stirred for an additional hour. The resulting suspension was filtered at 0° C., and the flask followed by second filter cake were washed with two 1 mL portions of Isopar C. The washings and second mother liquor were combined. The second filter cake was dried in a vacuum oven at 55° C. under house vacuum with a nitrogen gas sweep to give 0.8952 g of solids that contained a 46.47:53.53 (area/area) ratio of the (R)-enantiomer to the (S)-enantiomer by HPLC.

The combined second mother liquor and washings was concentrated in a 20 mL vial to a volume of 6 mL by rotary evaporation. The mixture was cooled to 0-5° C. and stirred for 1 hour. The resulting slurry was filtered, and the third filter cake was washed with 1 mL of Isopar C. The third filter cake was dried in a vacuum oven at 55° C. under house vacuum with a nitrogen gas sweep to give 1.464 g (29.3% yield from the racemate) of (S)-6-chloro-7-tert-butyl-2-trifluoromethyl-2H-chromene-3-carboxylic acid. HPLC analysis of the third filter cake and the third mother liquor therefrom indicated both contained (S)-6-chloro-7-tert-butyl-2-trifluoromethyl-2H-chromene-3-carboxylic acid in 100% enantiomeric purity (i.e., 100% e.e.). Upon sitting overnight, additional solids crystallized out of the third mother liquor.

Under the above-described HPLC conditions, the original 5.0 g lot of (R)- and (S)-6-chloro-7-tert-butyl-2-trifluoromethyl-2H-chromene-3-carboxylic acid, the (R)-enantiomer had a retention time of 4.6 minutes, and the (S)-enantiomer had a retention time of 6.5 minutes.

### Example 42a

In a screening enantioselective fractional crystallization of (R)- and (S)-6-chloro-7-tert-butyl-2-trifluoromethyl-2H-chromene-3-carboxylic acid, basic chiral auxiliaries were screened in ethanol according to the above-described method, and crystals of salts with brucine, cis-(1R,2S)-(+)-2-(benzylamino)cyclohexylmethanol, (R)-(+)-4-diphenylmethyl-2-oxazolidinone, and (1R,2S)-2-amino-1,2-diphenylethanol were observed. In another screening enantioselective fractional crystallization of (R)- and (S)-6-chloro-7-tert-butyl-2-trifluoromethyl-2H-chromene-3-carboxylic acid, basic chiral auxiliaries were screened in MTBE according to the above-described method, and crystals of salts with brucine, (1R,2S)-2-amino-1,2-diphenyl ethanol, (R)-(+)-4-diphenylmethyl-2-oxozolidinone, (R)-(+)-4-diphenylmethyl-2-oxozolidinone in EtOH, (1R,2S)-(+)-cis-[2-(benzylamine)cyclohexyl]methanol, quinine, (+)-cinchonine, (−)-cinchonidine, 0.5 eq of L-phenylalaninol with seeding, (R)-(−)-2-amino-1-butanol, (R)-(−)-phenylglycinol (gel like crystals), (1R,2R)-(+)-1,2-diphenylethylenediamine, and (S)-(−)-methylbenzylamine were observed.

### Example 42b

In an enantioselective fractional crystallization of (R)- and (S)-6-chloro-7-tert-butyl-2-trifluoromethyl-2H-chromene-3-carboxylic acid, the procedure of Example 42 is used except that n-heptane is used in place of Isopar C.

### Example 43

A first organic phase containing (S)-6-chloro-7-tert-butyl-2-trifluoromethyl-2H-chromene-3-carboxylic acid that was free of (+)-cinchonine was prepared according to the procedure described in Example 42, second and third paragraphs, except heptane was used instead of Isopar C to give the first organic phase comprised of MTBE-heptane (75:25, volume:volume). A 0.5 mL aliquot of the first organic phase was removed and rotary evaporated to dryness in order to determine the total amount of free 6-chloro-7-tert-butyl-2-trifluoromethyl-2H-chromene-3-carboxylic acid in the first organic phase. To the balance of the first organic phase was added 1.0 mole equivalent of L-phenylalaninol, and the resulting suspension was filtered and the filter cake was washed with heptane to give (S)-6-chloro-7-tert-butyl-2-trifluoromethyl-2H-chromene-3-carboxylic acid L-phenylalaninol salt in 32% recovery based on the determined total amount of free 6-chloro-7-tert-butyl-2-trifluoromethyl-2H-chromene-3-carboxylic acid in the first organic phase.

A sample of the filter cake was diluted with heptane-ethanol (90: 10, volume:volume) to a concentration of 0.5 mg of 6-chloro-7-tert-butyl-2-trifluoromethyl-2H-chromene-3-carboxylic acid per μL of solution and 10 μL were introduced onto a CHIRALPAK® AD column (4.6 mm inner diameter X 250 mm length), and eluted with mobile phase comprising heptane-ethanol-trifluoroacetic acid (95:5:0.1, volume proportions) with a UV detector at 254 nm and a flow rate at 1 mL/minute. HPLC analysis of the filter cake indicated a 94:6 ratio of the (S)-enantiomer to the (R)-enantiomer (area/area).

### Example 44

To a 500 mL 4-necked round bottom flask was added 10 g (34.2 mmol) of (R)- and (S)-6-chloro-8-methyl-2-trifluoromethyl-2H-chromene-3-carboxylic acid and 100 mL of MTBE. To the resulting mixture were added 7.20 g (34.2 mmol) of (S)-(−)-N-benzyl-α-methylbenzylamine, and the mixture was heated to 50° C. (a thin slurry formed). The mixture was cooled to room temperature, and 100 mL of heptane was added. The flask was covered to protect it from light, and the mixture was stirred for 16 hours at 15-25° C. The resulting suspension was filtered, and the flask followed by the resulting filter cake was washed with 25 mL of heptane. The filter cake was dried in a vacuum oven at 50° C. under house vacuum with nitrogen sweep for 16 hours to yield 6.37 g of (S)-6-chloro-8-methyl-2-trifluoromethyl-2H-chromene-3-carboxylic acid (S)-(−)-N-benzyl-α-methylbenzylamine salt (37% yield from the racemate). HPLC analysis of the first filter cake indicated a 97.72:2.28 ratio of the (S)-enantiomer to the (R)-enantiomer (area/area). HPLC analysis of the first mother liquor indicated a 79.62:20.38 ratio of the (R)-enantiomer to the (S)-enantiomer. (HPLC procedure: Dilute sample with 10% EtOH-heptane to final concentration of 0.5 mg/mL. Column: CHIRALPAK® AD. Mobile phase was heptane-ethanol-trifluoroacetic acid (95:5:0.1, volume proportions). Program was 9 minutes. Detector was at 254 nm. Flow rate was 1 mL/minute. The (R)-enantiomer eluted at approximately 5.2 minutes and the (S)-enantiomer eluted at approximately 6.2 minutes.)

To a 500 mL 4-necked round bottom flask was added 6.3 g of the (S)-6-chloro-8-methyl-2-trifluoromethyl-2H-chromene-3-carboxylic acid (S)-(−)-N-benzyl-α-methylbenzylamine salt and 63 mL of MTBE. The mixture was warmed to 40° C. to form a thin slurry. The mixture was cooled to room temperature, and 63 mL of heptane were added. The flask was covered to protect it from light, and the mixture was stirred for 16 hours at 15-25° C. The resulting suspension was filtered, and the flask followed by the resulting filter cake was washed with 25 mL of heptane. The second filter cake was dried for 24 hours in a vacuum oven at 50° C. under house vacuum with nitrogen sweep to yield 5.52 g (87.6% recovery, 32.4% overall yield from racemate) of (S)-6-chloro-8-methyl-2-trifluoromethyl-2H-chromene-3-carboxylic acid (S)-(−)-N-benzyl-α-methylbenzylamine salt. HPLC analysis of the second filter cake indicated a 99.46:0.54 ratio of the (S)-enantiomer to the (R)-enantiomer (area/area). HPLC analysis of the second mother liquor indicated a 90.54:9.46 ratio of the (R)-enantiomer to the (S)-enantiomer.

### Example 45

To a 20 mL 3-necked round bottom vial was added 500 mg (1.616 mmol) of (R)- and (S)-6-chloro-5,7-dimethyl-2-trifluoromethyl-2H-chromene-3-carboxylic acid and 10 mL of MTBE-CH₂Cl₂ (50:50, volume:volume). To the mixture was added 476 mg (1.616 mmol) of (+)-cinchonine, and the mixture was shaken to give a solution. The volume of the mixture was reduced by sweeping nitrogen gas into the vial until an oil formed. To the mixture was added 10 mL of MTBE, and the volume of the mixture was reduced until an oil formed as before. To the mixture was added another 10 mL of MTBE, and the volume of the mixture was reduced until an oil formed as before. To the mixture was added another 10 mL of MTBE and 5 mL of heptane, and the mixture was shaken for 16 hours at 15-25° C. using mechanical shaker. The resulting suspension was filtered at room temperature, and solvent was removed from the filter cake by applying a vacuum to filter funnel to give 547 mg (56% yield) of (S)-6-chloro-5,7-dimethyl-2-trifluoromethyl-2H-chromene-3-carboxylic acid (+)-cinchonine salt. HPLC analysis indicated an 85.6:14.4 ratio of the (S)-enantiomer to the (R)-enantiomer in the first filter cake and a 97.3:2.7 ratio of the (R)-enantiomer to the (S)-enantiomer in the first mother liquor. (HPLC: Sample prep: 1-2 mg of sample was added to 1 mL of MTBE and 1 mL of 1N HCl. The mixture was shaken and the phases were separated. The organic phase was blown to dryness under a nitrogen sweep. To the residue was added 2 mL of acetonitrile-water (50:50, volume:volume. About 10 μL was injected onto a CHIROBIOTIC® R 150 mm length×4.6 mm inner diameter column at 30° C. Mobile phase: buffer (1% triethylamine)-acetonitrile (78:22). 1% triethylamine buffer was prepared with 495 mL of H₂O, 5 mL of triethylamine, and adjusted pH to 4 with (approximately 9 μL) of glacial acetic acid. Runtime was 12 minutes. Flow rate was 1 mL/minute. UV detection was at 230 nm. The (R)-enantiomer eluted at approximately 3.2 minutes and the (S)-enantiomer eluted at approximately 4.1 minutes.

To a 20 mL vial was added 100 mg of the first filter cake and 2 mL of ethanol-MTBE (25:75, volume: volume) to form a slurry. The mixture was shaken for 4 hours at 15-25° C. The suspension was filtered at room temperature, and the vial followed by second filter cake was rinsed with 1 mL of ethanol-MTBE (25:75, volume: volume). The second filter cake was dried under vacuum to give 65.5 mg (65.5% recovery for the recrystallization and a 36.7% yield from the racemate) of (S)-6-chloro-5,7-dimethyl-2-trifluoromethyl-2H-chromene-3-carboxylic acid (+)-cinchonine salt. HPLC analysis indicated a 96.5:3.5 ratio of the (S)-enantiomer to the (R)-enantiomer in the second filter cake and a 41.2:58.8 ratio of the (R)-enantiomer to the (S)-enantiomer in the second mother liquor.

To a 20 mL vial was added 30 mg of the second filter cake and 0.6 mL of ethanol-MTBE (25:75, volume: volume) to form a slurry. The mixture was shaken for 16 hours at 15-25° C., and filtered at room temperature. The vial followed by third filter cake was rinsed with 1 mL of ethanol-MTBE (25:75, volume: volume). The third filter cake was dried under vacuum to give 19.9 mg (66.3% recovery for this recrystallization and an overall yield of 24.3% from the racemate). HPLC analysis of the third filter cake indicated a 98.4:1.6 ratio of the (S)-enantiomer to the (R)-enantiomer.

### Example 46

To a 500 mL 4-necked round bottom flask was added 10.0 g (36.7 mmol) of (R)- and (S)-6,8-dimethyl-2-trifluoromethyl-2H-chromene-3-carboxylic acid and 100 mL of MTBE. The mixture was stirred and warmed to 40° C. to form a solution. To this solution was added 9.31 g (44.1 mmol) of (S)-(−)-N-benzyl-α-methylbenzylamine, and the resulting mixture was cooled to 30° C. while stirring to yield a slightly turbid mixture. The mixture was further cooled to room temperature, and 100 mL of heptane were added. The flask was covered to protect it from light, and the mixture was stirred for 16 hours at 15-25° C. The resulting suspension was filtered, and the flask followed by the resulting filter cake was washed with 25 μL of heptane. The filter cake was dried in a vacuum oven at 50° C. under house vacuum with nitrogen sweep for 16 hours to yield 8.28 g of (S)-6,8-dimethyl-2-trifluoromethyl-2H-chromene-3-carboxylic acid (S)-(−)-N-benzyl-α-methylbenzylamine salt. HPLC analysis of the first filter cake indicated an 89.29:10.71 ratio of the (S)-enantiomer to the (R)-enantiomer (area/area). HPLC analysis of the first mother liquor indicated an 85.40:14.60 ratio of the (R)-enantiomer to the (S)-enantiomer. (HPLC procedure: Dilute sample with heptane-ethanol (90:10, volume:volume) to final concentration of 0.5 mg/mL. Column: CHIRALPAK®R AD. Mobile phase was heptane-ethanol-trifluoroacetic acid (95:5:0.1, volume proportions). Program was 9 minutes. Detector was at 254 nm. Flow rate was 1 mL/minute. The (R)-enantiomer eluted at approximately 5.0 minutes and the (S)-enantiomer eluted at approximately 6.1 minutes.

To a round bottom flask was added 7.9 g of the first filter cake and 79 mL of MTBE, and the mixture was warmed to 47.1° C. to form a thin slurry. The mixture was cooled to room temperature, and 79 mL of heptane were added. The flask was covered to protect it from light, and the mixture was stirred for 16 hours at 15-25° C. The resulting suspension was filtered, and the flask followed by the resulting filter cake were washed with 25 mL of heptane. The second filter cake was dried for 24 hours in a vacuum oven 50° C. under house vacuum with nitrogen sweep to give 6.79 g (86.0% recovery for the recrystallization and a 40.1% yield overall from the racemate) of (S)-6,8-dimethyl-2-trifluoromethyl-2H-chromene-3-carboxylic acid (S)-(−)-N-benzyl-α-methylbenzylamine salt. HPLC analysis of the second filter cake indicated 97.67:2.33 ratio of the (S)-enantiomer to the (R)-enantiomer. HPLC analysis of the second mother liquor indicated a 78.24:21.76 ratio of the (R)-enantiomer to the (S)-enantiomer.

### Example 47

To remove physical contamination in the starting material, 8.7 g of (R)- and (S)-6-trifluoromethoxy-8-ethyl-2-trifluoromethyl-2H-chromene-3-carboxylic acid were dissolved in 26.1 mL MTBE. The mixture was filtered through a 15 mL medium fritted glass filter, and the filter cake was washed with 2 mL of MTBE. The first filter cake was blown to dryness with a nitrogen sweep, and dried for 48 hours in a vacuum oven at 50° C. under house vacuum with a nitrogen sweep to give 8.46 g of (R)- and (S)-6-trifluoromethoxy-8-ethyl-2-trifluoromethyl-2H-chromene-3-carboxylic acid.

To a 250 mL 3-necked round bottom flask marked with a 20 mL pot volume line was added 4 g (11.2 mmol) of the decontaminated (R)- and (S)-6-trifluoromethoxy-8-ethyl-2-trifluoromethyl-2H-chromene-3-carboxylic acid and 40 mL of MTBE. To this mixture was added 2.34 mL (11.2 mmol) of (S)-(−)-N-benzyl-α-methylbenzylamine, and the mixture was stirred for 2 minutes to give a solution. To the solution was added 80 mL of heptane, and the mixture was distilled under vacuum to a volume at the 20 mL pot volume line. A white slurry formed. To the suspension was added 20 mL of heptane, and the mixture was stirred for 4 hours at 15-25° C. The suspension was filtered on a 15 mL course glass fritted filter, and the flask followed by the second filter cake were washed with two 5 mL heptane washes. The second filter cake was dried in a vacuum oven at 50° C. under house vacuum with nitrogen sweep for 72 hours to give 2.89 g (45% yield from the racemate) of (S)-6-trifluoromethoxy-8-ethyl-2-trifluoromethyl-2H-chromene-3-carboxylic acid (S)-(−)-N-benzyl-α-methylbenzylamine salt. HPLC analysis of the second filter cake indicated a 90.49:9.51 ratio (area/area) of the (S)-enantiomer to the (R)-enantiomer. HPLC analysis of second mother liquor indicated an 83.94:16.06 ratio of the (R)-enantiomer to the (S)-enantiomer. (HPLC procedure: Dilute sample with heptane-ethanol (90:10, volume:volume) to final concentration of 0.5 mg/mL. Column: CHIRALPAK® AD. Mobile phase was heptane-ethanol-trifluoroacetic acid (95:5:0.1, volume proportions). Program was 9 minutes. Detector was at 254 nm. Flow rate was 1 mL/minute. The (R)-enantiomer eluted at approximately 3.9 minutes and the (S)-enantiomer eluted at approximately 4.2 minutes.

To a 100 mL 3-necked round bottom flask was added 2.8 g of the second filter cake and 28 mL of heptane to form a slurry. The flask was covered to protect it from light, and the mixture was stirred for 4 hours at 15-25° C. with overhead stirrer set to 200 rpm. The suspension was filtered on a 30 mL course glass fritted filter. The flask followed by the third filter cake were washed with two 2 mL heptane washes. The third filter cake was dried for 24 hours in a vacuum oven at 50° C. under house vacuum with nitrogen sweep to give 2.37 g (84.6% yield for the recrystallization and a 38.5% yield overall from the racemate) of (S)-6-trifluoromethoxy-8-ethyl-2-trifluoromethyl-2H-chromene-3-carboxylic acid (S)-(−)-N-benzyl-α-methylbenzylamine salt. HPLC analysis of the third filter cake indicated a 99.15:0.85 ratio of the (S)-enantiomer to the (R)-enantiomer. HPLC analysis of the third mother liquor indicated an 80.15:19.85 ratio of the (R)-enantiomer to the (S)-enantiomer.

Enantiomeric excess for Examples (A) to (H) was determined by enantioselective high-pressure liquid chromatography (“HPLC”) using the HPLC method described below in Analytical Method (A).

### Analytical Method (A)

Using a column with 0.46 cm inner diameter and 250 mm length filled with CHIRALPAK® AD stationary phase, a 10 μL injection volume, by eluting at room temperature with mobile phase (volume proportions) 95%/5% heptane:ethanol with 0.1% trifluoroacetic acid, at room temperature, flow rate at 1 mL/minute isocratic, and detected with a photodiode array detector at 254 nm wavelength, and a run time of 10 minutes.

### Example (A)

A 1.0-mg/mL solution of (S)-6-chloro-7-tert-butyl-2-trifluoromethyl-2H-chromene-3-carboxylic acid in ethanol was placed in a quartz cuvette and irradiated with light from a UV spot lamp that produced a 5 mm diameter spot of UV light (320-390 nm wavelength) at 4 W/cm² intensity. After 30 minutes, an aliquot was analyzed by HPLC and was found to be a racemic mixture of (R)- and (S)-6-chloro-7-tert-butyl-2-trifluoromethyl-2H-chromene-3-carboxylic acid.

### Example (B)

A 50-mg/mL solution of (S)- and (R)-6-chloro-7-tert-butyl-2-trifluoromethyl-2H-chromene-3-carboxylic acid (21% e.e. of (S) enantiomer) in ethanol was divided into two aliquots. The first aliquot was placed in a quartz cuvette and irradiated in 90-second intervals over 25 minutes with light from a UV spot lamp that produced a 5 mm diameter spot of UV light (320-390 nm wavelength) at 4 W/cm² intensity to give a solution with 7.4% e.e. by HPLC. The second aliquot was placed in a quartz cuvette and irradiated continuously over 25 minutes with light from the UV spot lamp to give a solution with 12% e.e. by HPLC.

### Example (C)

A 40-mg/mL solution of (S)-6-chloro-7-tert-butyl-2-trifluoromethyl-2H-chromene-3-carboxylic acid in ethanol was placed in a quartz cuvette and irradiated with light from a UV spot lamp that produced a 5 mm diameter spot of UV light (320-390 nm wavelength) at 4 W/cm² intensity. Aliquots were taken at time=0, 1, 2, 4, 8, 12, and 16 minutes and analyzed by HPLC. The experiment was repeated. A rate constant k for each of the two experiments (k₁ and k₂) was calculated using the following equation:

$$k = \frac{ln ( e . e . ) - C}{- 2 t}$$

wherein t is the time in minutes, C is the concentration of chromene in moles per liter, and ln (e.e.) is the natural logarithm of percent enantiomeric excess. The rate constant k₁ was 0.0764/minute and k₂ was 0.0787/minute. A half-live τ for each of the two experiments (τ₁ and τ₂) was calculated using the following equation:

$$τ = \frac{ln ( 2 )}{2 k}$$

The half-life τ₁ was 4.54 minutes and τ₂ was 4.40 minutes.
A specific half-life of about 30 minutes per gram of enantiomer was calculated.

### Example (D)

The procedure of Example (C) was repeated except the concentration of (S)-6-chloro-7-tert-butyl-2-trifluoromethyl-2H-chromene-3-carboxylic acid in ethanol was 66.7-mg/mL. Aliquots were taken at time=0, 1, 2, 4, 8, 12, and 16 minutes and analyzed by HPLC. The natural logarithm of e.e. at each time point was determined for each aliquot. The half-life T was 9.81 minutes. The natural logarithm of e.e. data is provided below in Table 1 in the row labeled “ln (e.e.).”

TABLE 1
Time (minutes)

| | 0 | 1 | 2 | 4 | 8 | 12 | 16 |
|---|---|---|---|---|---|---|---|
| in (e.e.) | 4.4 | 4.3 | 4.2 | 4.0 | 3.8 | 3.4 | 3.3 |

### Example (E)

A weight of 16 g of (R)-6-chloro-7-tert-butyl-2-trifluoromethyl-2H-chromene-3-carboxylic acid was dissolved in 400 mL of ethanol to give a 40-g/L solution, and the solution was placed in a 400-mL photoreactor containing annular geometry having 450 W UV lamp located in the center of the reactor and separated from the reaction medium by a quartz tube. The mixture was irradiated and aliquots were taken at about time=0, 12, 24, 41, 60, 87, 105, 135, and 162 minutes and analyzed by HPLC. A rate constant k and half-life τ were calculated as above and found to be k=0.0109/minute and τ=31.8 minutes. A specific half-life of about 2.0 minutes per gram was calculated.

The natural logarithm of e.e. at each time point was determined for each aliquot. The natural logarithm of e.e. data is provided below in Table 2 in the row labeled “ln (e.e.).”

TABLE 2
Time (minutes)

| | 0 | 12 | 24 | 41 | 60 | 87 | 105 | 135 | 162 |
|---|---|---|---|---|---|---|---|---|---|
| In (e.e.) | 4.5 | 4.3 | 4.0 | 3.6 | 3.2 | 2.7 | 2.2 | 1.6 | 1.1 |

### Example (F)

Using the procedure of Example (E), additional photoracemization experiments were run with 4.00-g, 8.00-g, 10.00-g, 13.00-g, and 20.04-g of (R)-6-chloro-7-tert-butyl-2-trifluoromethyl-2H-chromene-3-carboxylic acid in 400 mL of ethanol to give concentrations of 10.0-mg/mL, 20.0-mg/mL, 25.0-mg/mL, 32.5-mg/mL, and 50.1-mg/mL, respectively. Half-lives (minutes) and specific half-lives (minutes per gram) were calculated for each concentration. The results are shown below in Table 3 along with the results from Example (E) in the columns labeled “τ (min.)” and “τ/m (min./g).”

TABLE 3

| Weight of | Concentration of | | |
|---|---|---|---|
| enantiomer | enantiomer | | |
| (g) | (mg/mL) | τ (min.) | τ/m (min./g) |
| 4.00 | 10.0 | 9.08 | 2.27 |
| 8.00 | 20.0 | 16.38 | 2.05 |
| 10.00 | 25.00 | 19.68 | 1.97 |
| 13.00 | 32.5 | 22.57 | 1.74 |
| 16.00 | 40.0 | 31.65 | 1.98 |
| 20.04 | 50.1 | 31.39 | 1.57 |

### Example (G)

Using the procedure of Example (E), 10-g of (R)-8-ethyl-6-trifluoromethoxy-2-trifluoromethyl-2H-chromene-3-carboxylic acid was dissolved in 400 mL of ethanol to a concentration of 25-mg/mL, and the mixture was filtered to remove a small amount of insoluble material. The filtrate was placed in the photoreactor and irradiated. Over the course of about 95 minutes, a decrease in ln (e.e.) from about 4.3 at t=5 minutes to about 1.4 at t=95 minutes was observed. Half-life τ was 20.7 minutes.

### Example (H)

Using the procedure of Example (E), 10-g of (R)-6,8-dimethyl-2-trifluoromethyl-2H-chromene-3-carboxylic acid was dissolved in 400 mL of ethanol to a concentration of 25-mg/mL, and the mixture was filtered to remove a small amount of insoluble material. The filtrate was placed in the photoreactor and irradiated. Over the course of about 105 minutes, a decrease in ln (e.e.) from about 4.2 at t=5 minutes to about 1.0 at t=105 minutes was observed. Half-life τ was 21.9 minutes.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All references cited above, including patents, patent application publications, and scientific journals, are hereby incorporated herein by reference in their entireties and for all purposes.

What is claimed is:

1. A method for separating enantiomers of a substituted 2-trifluoromethyl-2H-chromene-3-carboxylic acid or derivative thereof, the method comprising:
(a) introducing a mixture of the enantiomers to a chiral stationary phase; and
(b) eluting at least one of the enantiomers with a mobile phase;
wherein the substituted 2-trifluoromethyl-2H-chromene-3-carboxylic acid or derivative thereof is a compound of Formulas I″, I′, I, or II 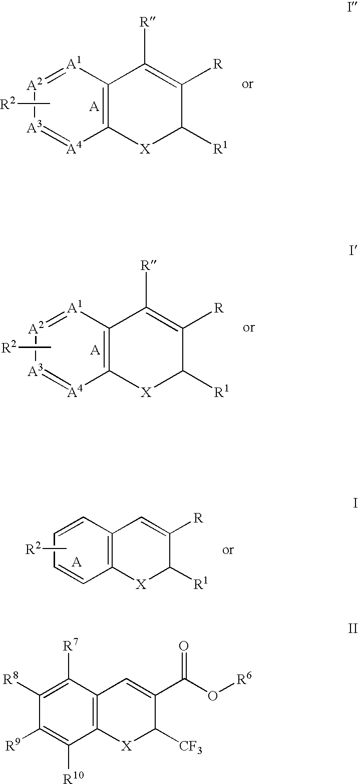
for Formula I″:
wherein X is selected from O, S, CR^{c}R^{b} and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₁-C₆-alkyl and C₂-C₆-alkenyl;
wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I′:
wherein X is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₂-C₆-alkynyl and C₂-C₆-alkenyl;
wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I:
wherein X is selected from O or S or NR^{a};
wherein R^{a} is alkyl;
wherein R is selected from carboxyl, aminocarbonyl, alkylsulfonylaminocarbonyl and alkoxycarbonyl;
wherein R¹ is selected from haloalkyl, alkyl, aralkyl, cycloalkyl and aryl optionally substituted with one or more radicals selected from alkylthio, nitro and alkylsulfonyl; and
wherein R² is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R² together with ring A forms a naphthyl radical;
for Formula II:
wherein X is selected from O, S, and NH;
wherein R⁶ is H or alkyl; and
wherein R⁷, R⁸, R⁹, and R¹⁰ independently are selected from H, alkenyl, alkoxy, alkoxyalkyl, alkoxycarbonylalkyl, alkyl, alkylamino, alkylcarbonyl, alkylheteroaryl, alkylsulfonylalkyl, alkylthio, alkynyl, aminocarbonylalkyl, aryl, arylalkenyl, arylalkoxy, arylalkyl, arylalkylamino, arylalkynyl, arylcarbonyl, aryloxy, cyano, dialkylamino, halo, haloalkoxy, haloalkyl, heteroaryl, heteroarylalkoxy, heteroarylcarbonyl, hydroxy and hydroxyalkyl; wherein each of aryl, wherever it occurs, is independently substituted with one to five substituents selected from the group consisting of alkyl, alkoxy, alkylamino, cyano, halo, haloalkyl, hydroxy, and nitro.

2. The method according to claim 1, the method further comprising a step of isolating in a form that is substantially free of mobile phase, at least one of the separated enantiomers.

3. The method according to claim 1, the method further comprising a step of monitoring the eluate produced in the eluting step for at least one of the enantiomers.

4. The method according to claim 1, wherein the mixture of the enantiomers comprises a substituted 2-trifluoromethyl-2H-chromene-3-carboxylic acid, a substituted 2-trifluoromethyl-1,2-dihydro-quinoline-3-carboxylic acid, a substituted 2-trifluoromethyl-2H-thiochromene-3-carboxylic acid, or a substituted 3-trifluoromethyl-3,4-dihydro-naphthalene-2-carboxylic acid and the mobile phase is:
a single polar solvent;
a solution comprising a polar solvent and an acidic solvent wherein the polar solvent is at least 99% volume/volume of the solution and the acidic solvent is less than 1% volume/volume of the solution; or
a solution comprising a polar solvent, an acidic solvent, and a nonpolar solvent wherein the polar solvent is less than or equal to 50% volume/volume of the mixture, the acidic solvent is less than 1% volume/volume of the solution and the nonpolar solvent is greater than 50% volume/volume of the solution.

5. The method according to claim 1, wherein the method comprises enantioselective steady state recycling chromatography or enantioselective multicolumn chromatography.

6. The method according to claim 1, the method further comprising a step of subjecting at least one of the separated enantiomers produced in the eluting step to enantioselective fractional crystallization.

7. The method according to claim 1, wherein the mixture of the enantiomers comprises a compound of Formula II wherein X is O and R⁶ is H.

8. The method according to claim 1, wherein the mixture of the enantiomers comprises:
(R)- and (S)-6-chloro-8-methyl-2-trifluoromethyl-2H-chromene-3-carboxylic acid;
(R)- and (S)-6-chloro-5,7-dimethyl-2-trifluoromethyl-2H-chromene-3-carboxylic acid;
(R)- and (S)-6,8-dimethyl-2-trifluoromethyl-2H-chromene-3-carboxylic acid; or
(R)- and (S)-8-ethyl-6-trifluoromethoxy-2-trifluoromethyl-2H-chromene-3-carboxylic acid.

9. The method according to claim 1, wherein the mixture of the enantiomers comprises (R)- and (S)-6-chloro-7-tert-butyl-2-trifluoromethyl-2H-chromene-3-carboxylic acid.

10. A method for separating enantiomers of a substituted 2-trifluoromethyl-2H-chromene-3-carboxylic acid or derivative thereof, the method comprising:
(a) subjecting a mixture of the enantiomers to enantioselective fractional crystallization to give crystals and mother liquor; and
(b) isolating at least one of the enantiomers from the crystals or mother liquor;
wherein the substituted 2-trifluoromethyl-2H-chromene-3-carboxylic acid or derivative thereof is a compound of Formulas I″, I′, I, or II 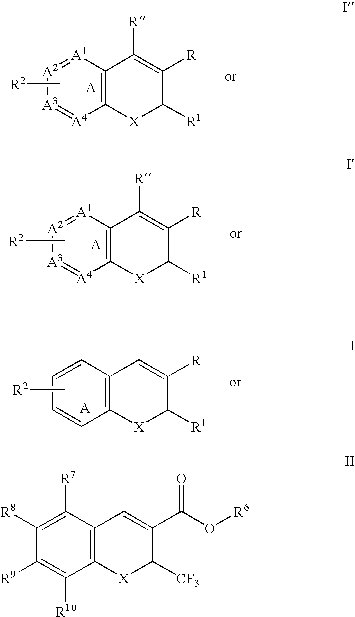
for Formula I″:
wherein X is selected from O, S, CR^{c}R^{b} and NR^{a};
wherein R^{a} is selected from hydrido, C₁-C₃-alkyl, (optionally substituted phenyl)-C₁-C₃-alkyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a 3-6 membered cycloalkyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₁-C₆-alkyl and C₂-C₆-alkenyl;
wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon;
or wherein R² together with ring A forms a radical selected from naphthyl, quinolyl, isoquinolyl, quinolizinyl, quinoxalinyl and dibenzofuryl;
for Formula I′:
wherein X is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl;
or wherein CR^{c}R^{b} form a cyclopropyl ring;
wherein R is selected from carboxyl, aminocarbonyl, C₁-C₆-alkylsulfonylaminocarbonyl and C₁-C₆-alkoxycarbonyl;
wherein R″ is selected from hydrido, phenyl, thienyl, C₂-C₆-alkynyl and C₂-C₆-alkenyl;
wherein R¹ is selected from C₁-C₃-perfluoroalkyl, chloro, C₁-C₆-alkylthio, C₁-C₆-alkoxy, nitro, cyano and cyano-C₁-C₃-alkyl;
wherein R² is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A A², A³ and A⁴ are carbon;
or wherein R² together with ring A forms a radical selected from naphthyl, quinolyl, isoquinolyl, quinolizinyl, quinoxalinyl and dibenzofuryl;
for Formula I:
wherein X is selected from O or S or NR^{a};
wherein R^{a} is alkyl;
wherein R is selected from carboxyl, aminocarbonyl, alkylsulfonylaminocarbonyl and alkoxycarbonyl;
wherein R¹ is selected from haloalkyl, alkyl, aralkyl, cycloalkyl and aryl optionally substituted with one or more radicals selected from alkylthio, nitro and alkylsulfonyl; and
wherein R² is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;
or wherein R² together with ring A forms a naphthyl radical;
for Formula II:
wherein X is selected from O, S, and NH;
wherein R⁶ is H or alkyl; and
wherein R⁷, R⁸, R⁹, and R¹⁰ independently are selected from H, alkenyl, alkoxy, alkoxyalkyl, alkoxycarbonylalkyl, alkyl, alkylamino, alkylcarbonyl, alkylheteroaryl, alkylsulfonylalkyl, alkylthio, alkynyl, aminocarbonylalkyl, aryl, arylalkenyl, arylalkoxy, arylalkyl, arylalkylamino, arylalkynyl, arylcarbonyl, aryloxy, cyano, dialkylamino, halo, haloalkoxy, haloalkyl, heteroaryl, heteroarylalkoxy, heteroarylcarbonyl, hydroxy and hydroxyalkyl; wherein each of aryl, wherever it occurs, is independently substituted with one to five substituents selected from the group consisting of alkyl, alkoxy, alkylamino, cyano, halo, haloalkyl, hydroxy, and nitro.

11. The method according to claim 10, wherein the crystals comprise a (S)-(−)-α-methylbenzylamine, (−)-cinchonidine, (S)-(−)-2-amino-3-phenyl-1-propanol, (+)-brucine, (1R,2S)-2-amino-1,2-diphenyl ethanol, (R)-(+)₄-diphenylmethyl-2-oxozolidinone, (1R,2S)-(+)-cis-[2 -(benzylamine)cyclohexyl]methanol, (+)-quinine, (+)-cinchonine, L-phenylalaninol, (R)-(−)-2-amino-1-butanol, (R)-(−)-phenylglycinol, (1R,2R)-(+)-1,2-diphenylethylenediamine, (1S,2R)-(+)-norephedrine, (1R,2S)-(−)-N-methylephedrine, (1R,2S)-(−)-ephedrine, (+)-quinidine, (1R,2S)-(+)-1-amino-2-indanol, (1R,2R)-(−)-2-amino-1-(4-nitrophenyl)-1,3-propanediol, (R)-(+)-N-benzyl-α-methylbenzyl amine, (+)-strychnine, (+)-dehydroabietylamine, (+)-amphetamine, (+)-deoxyphedrine, (+)-chloramphenicol intermediate, (+)-1-(1-naphthyl)ethylamine, (R)-(+)-α-methylbenzylamine, (+)-cinchonidine, (R)-(+)-2-amino-3-phenyl-1-propanol, (−)-brucine, (1S,2R)-2-amino-1,2-diphenyl ethanol, (S)-(−)-4-diphenylmethyl-2-oxozolidinone, (1S,2R)-(−)-cis-[2-(benzylamine)cyclohexyl]methanol, (−)-quinine, (−)-cinchonine, D-phenylalaninol, (S)-(+)-2-amino-1-butanol, (S)-(+)-phenylglycinol, (1S,2S)-(−)-1,2-diphenylethylenediamine, (1R,2S)-(−)-norephedrine, (1S, 2R)-(+)-N-methylephedrine, (1S,2R)-(+)-ephedrine, (−)-quinidine, (1S, 2R)-(−)-1-amino-2-indanol, (1S,2S)-(+)-2-amino-1-(4-nitrophenyl)-1,3-propanediol, (S)-(−)-strychnine, (−)-dehydroabietylamine, (−)-amphetamine, (−)-deoxyphedrine, (−)-chloramphenicol intermediate, or (−)-1-(1-napthyl)ethylamine salt of at least one enantiomer.

12. The method according to claim 11, wherein the crystals comprise:
(S)-6-chloro-8-methyl-2-trifluoromethyl-2H-chromene-3-carboxylic acid (S)-(−)-N-benzyl-α-methylbenzylamine salt;
(S)-6-chloro-5,7-dimethyl-2-trifluoromethyl-2H-chromene-3-carboxylic acid (+)-cinchonine salt;
(S)-6,8-dimethyl-2-trifluoromethyl-2H-chromene-3-carboxylic acid (S)-(−)-N-benzyl-α-methylbenzylamine salt;
(S)-8-ethyl-6-trifluoromethoxy-2-trifluoromethyl-2H-chromene-3-carboxylic acid (S)-(−)-N-benzyl-α-methylbenzylamine salt;
(R)-6-chloro-8-methyl-2-trifluoromethyl-2H-chromene-3-carboxylic acid (R)-(+)-N-benzyl-α-methylbenzylamine salt;
(R)-6-chloro-5,7-dimethyl-2-trifluoromethyl-2H-chromene-3-carboxylic acid (−)-cinchonine salt;
(R)-6,8-dimethyl-2-trifluoromethyl-2H-chromene-3-carboxylic acid (R)-(+)-N-benzyl-α-methylbenzylamine salt; or
(R)-8-ethyl-6-trifluoromethoxy-2-trifluoromethyl-2H-chromene-3-carboxylic acid (R)-(+)-N-benzyl-α-methylbenzylamine salt.

13. The method according to claim 11, wherein the crystals comprise:
(R)-6-chloro-7-tert-butyl-2-trifluoromethyl-2H-chromene-3-carboxylic acid (+)-cinchonine salt;
(S)-6-chloro-7-tert-butyl-2-trifluoromethyl-2H-chromene-3-carboxylic acid (−)-cinchonine salt;
(S)-6-chloro-7-tert-butyl-2-trifluoromethyl-2H-chromene-3-carboxylic acid L-phenylalaninol salt; or
(R)-6-chloro-7-tert-butyl-2-trifluoromethyl-2H-chromene-3-carboxylic acid D-phenylalaninol salt.
